(19) United States
(12) Patent Application Publication
(10) Pub. No.: US 2020/0405225 A1
(43) Pub. Date: Dec. 31, 2020

(54) METHODS AND SYSTEMS FOR IDENTIFYING OR MONITORING LUNG DISEASE

(71) Applicant: Veracyte, Inc., South San Francisco, CA (US)

(72) Inventors: Giulia C. KENNEDY, San Francisco, CA (US); Bonnie H. ANDERSON, Half Moon Bay, CA (US)

(21) Appl. No.: 16/696,888

(22) Filed: Nov. 26, 2019

Related U.S. Application Data
(63) Continuation of application No. PCT/US2018/035702, filed on Jun. 1, 2018.
(60) Provisional application No. 62/514,595, filed on Jun. 2, 2017.
(60) Provisional application No. 62/546,936, filed on Aug. 17, 2017.

(51) Int. Cl. A61B 5/00; G16H 50/20; G16H 50/30

(52) U.S. Cl. CPC A61B 5/4842; G16H 20/10; G16H 50/30; G16H 50/20

(57) ABSTRACT

Provided herein are methods, systems, and kits for improving the current clinical pathway of care for lung conditions using genomic classifiers at various decision points within the existing pathway to minimize unnecessary invasive procedures, enhance early detection and disease recurrence, and monitor efficacy of interventive therapies for prevention or disease reversal.

### CROSS-REFERENCE

This application is a continuation application of International Patent Application No. PCT/US2018/035702, filed on Jun. 1, 2018; which claims priority to U.S. provisional application 62/514,595 filed on Jun. 2, 2017 and U.S. provisional application 62/546,936 filed on Aug. 17, 2017, each of which is entirely incorporated herein by reference.

### BACKGROUND

There are methods currently available for detecting lung conditions, such as lung cancer. Such current clinical pathway of care for lung conditions suffer from a high rate of unnecessary invasive procedures, an inability to detect early lung conditions, or assess subject risk for developing a lung condition.

### SUMMARY

The present disclosure provides methods and systems for determining whether a subject has or is at risk of having a lung condition, such as, for example, lung cancer. Methods of the present disclosure may permit a subject to be screened or monitored for a progression or regression of the lung condition, in some cases using a sample non-invasively obtained from the subject (e.g., a nasal tissue sample). This may advantageously be used to screen for subjects that as asymptomatic for the lung condition, but who may otherwise be at risk of developing the lung condition (e.g., subjects exposed to cigarette smoke or air pollution), or to monitor subjects that have or are suspected of having the lung condition.

An aspect of the present disclosure provides a method for screening a subject for a lung condition, the method comprising (a) assaying epithelial tissue from a first sample obtained from a subject that has been (1) computer analyzed for a presence of one or more risk factors for developing the lung condition and (2) identified with the presence of the one or more risk factors, to identify a presence or absence of one or more biomarkers associated with a risk of developing the lung condition in the first sample; and; and (b) upon identifying the presence or absence of the one or more biomarkers, (i) directing an electronic imaging scan of a lung region of the subject to be obtained, which lung region is suspected of having the lung condition, or (ii) assaying other epithelial tissue from a second sample of the subject. In some embodiments, the method further comprises, prior to (b), receiving a request to assay the first sample comprising the epithelial tissue of the subject.

In some embodiments, the electronic imaging scan is a low-dose computerized tomography (LDCT) scan or magnetic resonance imaging (MM). In some embodiments, the LDCT scan provides a radiation exposure to the subject of less than about 5 millisieverts (mSv).

In some embodiments, the lung condition is lung cancer, chronic obstructive pulmonary disease (COPD), interstitial lung disease (ILD), or any combination thereof. In some embodiments, the lung condition is a lung cancer and the lung cancer comprises: a non-small cell lung cancer; an adenocarcinoma; a squamous cell carcinoma; a large cell carcinoma; a small cell lung cancer; or any combination thereof.

In some embodiments, the first sample or the second sample is obtained by a bronchoscopy. In some embodiments, the first sample or the second sample is obtained by fine needle aspiration. In some embodiments, the first sample or the second sample comprises a mucous epithelial tissue, a nasal epithelial tissue, a lung epithelial tissue, or any combination thereof. In some embodiments, the first sample or the second sample comprises epithelial tissue obtained along an airway of the subject.

In some embodiments, a portion of the first sample or the second sample is subjected to cytological testing that identifies the sample as ambiguous or suspicious. In some embodiments, upon identifying the first sample or the second sample as ambiguous or suspicious, performing (b) on a second portion of the sample, which second portion comprises the epithelial tissue.

In some embodiments, the second sample is different from the first sample. In some embodiments, the second sample is a different sample type from the first sample. In some embodiments, the first sample is obtained from the subject at a first time point and the second sample is obtained from the subject at a second time point, and the second time point is after the first time point. In some embodiments, the second time point is within about 1-2 years of the first time point.

In some embodiments, (a) comprises comparing the presence or absence of the one or more biomarkers to a reference set of one or more biomarkers. In some embodiments, the subject is in need of a treatment for the lung condition. In some embodiments, the subject is suspected of having an increased risk for developing a lung condition. In some embodiments, the subject is asymptomatic with respect to the lung condition. In some embodiments, the subject has not previously received the electronic imaging scan. In some embodiments, the subject has not previously received a definitive diagnosis.

In some embodiments, the one or more risk factors comprise: smoking; exposure to environmental smoke; exposure to radon; exposure to air pollution; exposure to radiation; exposure to an industrial substance; inherited or environmentally-acquired gene mutations; a subject's age; a subject having a secondary health condition; or any combination thereof. In some embodiments, the subject has two or more risk factors.

In some embodiments, the one or more biomarkers comprise at least five biomarkers. In some embodiments, the one or more biomarkers comprise one or more of: a gene or fragment thereof; a sequence variant; a fusion; a mitochondrial transcript; an epigenetic modification; a copy number variation; a loss of heterozygosity (LOH); or any combination thereof. In some embodiments, the presence or absence of the one or more biomarkers comprises a level of expression.

In some embodiments, the method identifies whether the subject is at an increased risk for developing the lung condition. In some embodiments, the identifying of (b) comprises employing a trained algorithm. In some embodiments, the trained algorithm is trained by a training set comprising epithelial cells obtained from an airway of an individual. In some embodiments, the trained algorithm is trained by a training set comprising samples benign for the lung condition and samples malignant for the lung condition. In some embodiments, the trained algorithm is trained by a training set comprising samples obtained from subjects having one or more risk factors.

In some embodiments, the method further comprises, prior to (a), computer analyzing the subject to identify the presence of said one or more risk factors in the subject for developing the lung condition.

Another aspect of the present disclosure provides a method for monitoring a subject having or suspected of having a lung condition. The method comprises (a) assaying a first sample comprising epithelial tissue obtained from a subject suspected of having the lung condition to identify a presence or an absence of one or more biomarkers associated with the lung condition, wherein the subject has previously received a positive indication of a presence of one or more lung nodules; and (b) upon identifying the presence or absence of the one or more biomarkers, (i) obtaining a second sample from the subject or (ii) directing the subject to obtain an electronic imaging scan of a lung region of the subject based on a result from (a).

In some embodiments, the positive indication is previously identified by an electronic imaging scan. In some embodiments, the electronic imaging scan is a low-dose computerized tomography (LDCT) scan or magnetic resonance imaging (MM). In some embodiments, the LDCT scan provides a radiation exposure to the subject of less than about 5 millisieverts (mSv).

In some embodiments, the one or more lung nodules is at least two nodules. In some embodiments, the obtaining the second sample from the subject comprises performing a bronchoscopy, a transthoracic needle aspiration (TTNA), or a video-assisted thorascopic surgery (VATS) on the subject. In some embodiments, the obtaining the second sample from the subject comprises performing a tissue biopsy.

In some embodiments, the presence or absence of the one or more biomarkers identifies the subject as high-risk or as low-risk of having the lung condition. In some embodiments, (b) further comprises recommending (i) or (ii) depending on an assessed risk.

In some embodiments, the lung condition is lung cancer, chronic obstructive pulmonary disease (COPD), interstitial lung disease (ILD), or any combination thereof. In some embodiments, the lung condition is a lung cancer and the lung cancer comprises: a non-small cell lung cancer; an adenocarcinoma; a squamous cell carcinoma; a large cell carcinoma; a small cell lung cancer; or any combination thereof.

In some embodiments, the first sample or the second sample is obtained by a bronchoscopy. In some embodiments, the first sample or the second sample is obtained by fine needle aspiration. In some embodiments, the first sample or the second sample comprises a mucous epithelial tissue, a nasal epithelial tissue, a lung epithelial tissue, or any combination thereof. In some embodiments, the first sample or the second sample comprises epithelial tissue obtained along an airway of the subject.

In some embodiments, the second sample is different from the first sample. In some embodiments, the second sample is a different sample type from the first sample. In some embodiments, the second sample is obtained from the subject at a time period later in time than the first sample is obtained from the subject. In some embodiments, the time period is from about 1 year to about 2 years.

In some embodiments, (b) comprises comparing the presence or absence of the one or more biomarkers to a reference set of one or more biomarkers. In some embodiments, the subject is a subject in need of a treatment for the lung condition. In some embodiments, the subject is suspected of having an increased risk for developing a lung condition. In some embodiments, the subject is asymptomatic for the lung condition. In some embodiments, the subject has not previously received a definitive diagnosis.

In some embodiments, the one or more biomarkers comprise at least five biomarkers. In some embodiments, the one or more biomarkers comprise one or more of: a gene or fragment thereof; a sequence variant; a fusion; a mitochondrial transcript; an epigenetic modification; a copy number variation; a loss of heterozygosity (LOH); or any combination thereof. In some embodiments, the presence or absence of the one or more biomarkers comprises a level of expression.

In some embodiments, the method identifies whether the subject is at an increased risk of having the lung condition. In some embodiments, the identifying of (a) comprises employing a trained algorithm. In some embodiments, the trained algorithm is trained by a training set comprising epithelial cells obtained from an airway of an individual. In some embodiments, the trained algorithm is trained by a training set comprising samples benign for the lung condition and samples malignant for the lung condition. In some embodiments, the trained algorithm is trained by a training set comprising samples obtained from subjects having one or more risk factors. In some embodiments, the method further comprises analyzing a blood sample from the subject, performing an electronic imaging scan on the subject, or a combination thereof.

In some embodiments, the second sample is a sample of epithelial, and wherein subsequent to (b), the sample of epithelial tissue is assayed for a presence or absence of one or more additional biomarkers. In some embodiments, the one or more additional biomarkers are the one or more biomarkers.

Another aspect of the present disclosure provides a method for monitoring a subject having or suspected of having a lung condition wherein the subject has previously received a recommendation to complete an interventive therapy for preventing or reversing the lung condition. The method comprises (a) subsequent to the subject completing at least a portion of the interventive therapy for the lung condition, assaying a first sample comprising epithelial tissue obtained from the subject to generate genetic data; (b) processing the genetic data to identify a presence or absence of one or more biomarkers associated with the lung condition; and (c) computer generating a report comprising a recommendation that a second sample be obtained from the subject.

Another aspect of the present disclosure provides a method. The method comprises (a) assaying a first sample comprising epithelial tissue obtained from a subject and identifying a presence or absence of one or more biomarkers, wherein the subject has previously received a recommendation to complete an interventive therapy for preventing or reversing a lung condition; and (b) upon completing at least a portion of the interventive therapy for the lung condition, obtaining a second sample from the subject and repeating (a) with the second sample.

In some embodiments, the method identifies subject compliance to the interventive therapy. In some embodiments, the method identifies efficacy of the interventive therapy to preventing or reversing the lung condition. In some embodiments, the interventive therapy comprises administering a pharmaceutical composition to the subject. In some embodiments, the pharmaceutical composition comprises a chemotherapeutic. In some embodiments, the interventive therapy comprises an exercise regime, a dietary regime, a reduction or omission of smoking, or any combination thereof.

In some embodiments, the lung condition is lung cancer, chronic obstructive pulmonary disease (COPD), interstitial lung disease (ILD), or any combination thereof. In some embodiments, the lung condition is a lung cancer and the lung cancer comprises: a non-small cell lung cancer; an adenocarcinoma; a squamous cell carcinoma; a large cell carcinoma; a small cell lung cancer; or any combination thereof.

In some embodiments, the first sample or the second sample is obtained by a bronchoscopy. In some embodiments, the first sample or the second sample is obtained by fine needle aspiration. In some embodiments, the first sample or the second sample comprises a mucous epithelial tissue, a nasal epithelial tissue, a lung epithelial tissue, or any combination thereof. In some embodiments, the first sample or the second sample comprises epithelial tissue obtained along an airway of the subject.

In some embodiments, the second sample is different from the first sample. In some embodiments, the second sample is a different sample type from the first sample. In some embodiments, the second sample is obtained from the subject at a time period later in time than the first sample is obtained from the subject. In some embodiments, the time period is from about 1 year to about 2 years.

In some embodiments, (a) comprises comparing the presence or absence of the one or more biomarkers to a reference set of one or more biomarkers. In some embodiments, the subject is a subject in need of a treatment for the lung condition. In some embodiments, the subject is suspected of having an increased risk for developing a lung condition. In some embodiments, the subject is asymptomatic with respect to the lung condition. In some embodiments, the subject has not previously received a definitive diagnosis.

In some embodiments, the one or more biomarkers comprise at least five biomarkers. In some embodiments, the one or more biomarkers comprise one or more of: a gene or fragment thereof; a sequence variant; a fusion; a mitochondrial transcript; an epigenetic modification; a copy number variation; a loss of heterozygosity (LOH); or any combination thereof. In some embodiments, the presence or absence of the one or more biomarkers comprises a level of expression.

In some embodiments, the identifying of (a) comprises employing a trained algorithm. In some embodiments, the trained algorithm is trained by a training set comprising epithelial cells obtained from an airway of an individual. In some embodiments, the trained algorithm is trained by a training set comprising samples benign for the lung condition and samples malignant for the lung condition. In some embodiments, the trained algorithm is trained by a training set comprising samples obtained from subjects having one or more risk factors. In some embodiments, the method further comprises analyzing a blood sample from the subject, performing an electronic imaging scan on the subject, or a combination thereof.

In some embodiments, (b) comprises processing the genetic data to identify an expression level corresponding to each of the one or more biomarkers. In some embodiments, (b) comprises processing the genetic data to identify at least one genetic aberration in the one or more biomarkers.

Another aspect of the present disclosure provides a method for monitoring the subject for a lung condition. The method comprises (a) assaying a first sample comprising epithelial tissue obtained from a subject and identifying a presence or absence of one or more biomarkers, wherein the subject has previously initiated a treatment for a lung condition; and (b) upon receiving a confirmation of remission, obtaining a second sample from the subject and repeating (a) with the second sample.

In some embodiments, the method identifies early stage lung condition recurrence through non-invasive monitoring. In some embodiments, the lung condition is lung cancer, chronic obstructive pulmonary disease (COPD), interstitial lung disease (ILD), or any combination thereof. In some embodiments, the lung condition is a lung cancer and the lung cancer comprises: a non-small cell lung cancer; an adenocarcinoma; a squamous cell carcinoma; a large cell carcinoma; a small cell lung cancer; or any combination thereof.

In some embodiments, the first sample or the second sample is obtained by a bronchoscopy. In some embodiments, the first sample or the second sample is obtained by fine needle aspiration. In some embodiments, the first sample or the second sample comprises a mucous epithelial tissue, a nasal epithelial tissue, a lung epithelial tissue, or any combination thereof. In some embodiments, the first sample or the second sample comprises epithelial tissue obtained along an airway of the subject.

In some embodiments, the second sample is different from the first sample. In some embodiments, the second sample is a different sample type from the first sample. In some embodiments, the second sample is obtained from the subject at a time period later in time than the first sample is obtained from the subject. In some embodiments, the time period is from about 1 year to about 2 years.

In some embodiments, (a) comprises comparing the presence or absence of the one or more biomarkers to a reference set of one or more biomarkers. In some embodiments, the subject is a subject in need of a treatment for the lung condition. In some embodiments, the subject is suspected of having an increased risk for a recurrence of the lung condition. In some embodiments, the subject is asymptomatic with respect to the lung condition.

In some embodiments, the one or more biomarkers comprise at least five biomarkers. In some embodiments, the one or more biomarkers comprise one or more of: a gene or fragment thereof a sequence variant; a fusion; a mitochondrial transcript; an epigenetic modification; a copy number variation; a loss of heterozygosity (LOH); or any combination thereof. In some embodiments, the presence or absence of the one or more biomarkers comprises a level of expression.

In some embodiments, the identifying of (a) comprises employing a trained algorithm. In some embodiments, the trained algorithm is trained by a training set comprising epithelial cells obtained from an airway of an individual. In some embodiments, the trained algorithm is trained by a training set comprising samples benign for the lung condition and samples malignant for the lung condition. In some embodiments, the trained algorithm is trained by a training set comprising samples obtained from subjects having one or more risk factors. In some embodiments, the method further comprises analyzing a blood sample from the subject, performing an electronic imaging scan on the subject, or a combination thereof. Another aspect of the present disclosure provides a method for monitoring a subject having or suspected of having a lung condition. The method comprises (a)

assaying a first sample comprising epithelial tissue obtained from a subject suspected of having the lung condition to identify a presence or absence of one or more biomarkers associated with the lung condition, wherein the subject has previously received a negative indication of a presence of a lung nodule; and (b) upon identifying the presence or absence of the one or more biomarkers, (i) obtaining a second sample from the subject or (ii) directing the subject to obtain an electronic imaging scan of a lung region of the subject based on a result from (a). In some embodiments, the method further comprises, prior to (a), computer analyzing the subject for a presence of one or more risk factors for developing the lung condition, and identifying the subject with the presence of the one or more risk factors.

Another aspect of the present disclosure provides a system for screening a subject for a lung condition. The system comprises one or more computer databases comprising health or physiological data of a subject; and one or more computer processors that are individually or collectively programmed to (i) analyze the health or physiological data for a presence of one or more risk factors for the subject developing the lung condition, and (2) upon identifying the one or more risk factors, generate a recommendation that epithelial tissue from a sample of the subject be assayed for one or more biomarkers associated with a risk of developing the lung condition.

Another aspect of the present disclosure provides a system for screening a subject for a lung condition. The system comprises one or more computer databases comprising (i) a first data set comprising data indicative of a presence of one or more risk factors for the subject developing the lung condition, and (ii) a second data set comprising data indicative of a presence or absence of one or more biomarkers in epithelial tissue in a sample of the subject, which one or more biomarkers are associated with a risk of developing the lung condition; and one or more computer processors that are individually or collectively programmed to (i) analyzing the first data set to identify the presence of the one or more risk factors, (ii) analyzing the second data set to identify the presence or absence of the one or more biomarkers, and (iii) upon identifying the presence or absence of the one or more biomarkers, generate a report that (1) directs an electronic imaging scan of a lung region of the subject to be obtained, which lung region is suspected of exhibiting the lung condition, or (2) directs other epithelial tissue from a second sample of the subject to be assayed.

Another aspect of the present disclosure provides a system for monitoring a subject having or suspected of having a lung condition. The system comprises one or more computer databases comprising a data set comprising data indicative of a presence or absence of one or more biomarkers in epithelial tissue in a first sample of the subject, which one or more biomarkers are associated with the lung condition; and one or more computer processors that are individually or collectively programmed to (i) determine that the subject has previously received a positive indication of a presence of one or more lung nodules, (ii) subsequent to (i), process the data set to identify the presence or absence of the one or more biomarkers, and (iii) upon identifying the presence or absence of the one or more biomarkers, generate a report that (1) directs a second sample to be obtained from the subject, or (2) directs another electronic imaging scan of a lung region of the subject to be obtained.

Another aspect of the present disclosure provides a system for monitoring a subject having or suspected of having a lung condition wherein the subject has previously received a recommendation to complete an interventive therapy for preventing or reversing the lung condition. The system comprises one or more computer databases comprising a data set comprising genetic data; and one or more computer processors that are individually or collectively programmed to (i) subsequent to the subject completing at least a portion of the interventive therapy for the lung condition, process the genetic data to identify a presence or absence of one or more biomarkers associated with the lung condition, and (iii) generate a report comprising a recommendation that a second sample be obtained from the subject.

Another aspect of the present disclosure provides a non-transitory computer readable medium comprising machine-executable code that, upon execution by one or more computer processors, implements any of the methods above or elsewhere herein.

Another aspect of the present disclosure provides a computer system comprising one or more computer processors and memory coupled thereto. The memory comprises a non-transitory computer-readable medium comprising machine-executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a diagram highlighting the clinical challenges of lung cancer diagnosis.

FIG. 2 shows the benefit of integrating methods that include genomic classifier analysis into the clinical pathway of care for lung cancer.

FIG. 3 shows an improved clinical decisions pathway which includes a genomic classifier analysis.

FIG. 4 shows the benefit of integrating methods that include genomic classifier analysis into the clinical pathway of care with a 47% reduction in procedure recommendations.

FIG. 5 shows the benefit of integrating methods that include genomic classifier analysis into the clinical pathway of care for idiopathic pulmonary fibrosis (IPF).

FIG. 6 shows a positive change in treatment decision by integrating genomic classifier analysis into the clinical pathway of care to differentiate usual interstitial pneumonia (UIP) from other interstitial lung disease (ILD) pathologies.

FIG. 7 shows the etiologic field of injury shares common pathways.

FIG. 8 shows an example of the difference between field of cancerization and the field of injury in a subject.

FIG. 9 shows a molecular view of the field of injury and field of cancerization.

FIG. 10 shows a standard clinical pathway of care for lung cancer improved by inclusion of a genomic classifier analysis (Bronchial Genomic Classifier).

FIG. 11a-b shows an improved clinical pathway of care for lung cancer by inclusion of multiple genomic classifier analysis (Bronchial Genomic Classifier; Nasa-Detect; Nasa-Risk Stratifier; Nasa-Protect Monitoring; Nasa-Recurrence).

FIG. 12 shows test characteristics of the Nasa-Detect classifier.

FIG. 13 shows test characteristics of the Nasa-Risk Stratifier classifier.

FIG. 14 shows test characteristics of the Nasa-Protect classifier.

FIG. 15 shows test characteristics of the Nasa-Recurrence classifier.

FIG. 16 shows evaluation of genomics in practice and prevention.

FIG. 17 shows an example of the samples characteristics and sample types used in the methods described herein.

FIG. 18 shows different subject cohorts with nasal/bronchial brushing samples.

FIG. 19 shows examples of training samples used to train a genomic classifier, such as the Nasa-Detect classifier.

FIG. 20 shows examples of training samples used to train a genomic classifier, such as the Nasa-Risk Stratifier classifier.

FIG. 21 shows types of biomarkers and the technology platforms used to detect different types of biomarkers.

FIG. 22 shows an example of RNA sequencing for genomic classifiers.

FIG. 23 shows an example of RNA sequencing.

FIG. 24 shows a flow diagram of a training and validation of a genomic classifier comprising a trained algorithm.

FIG. 25 shows an example of the diverse cytological and histological subtypes employed in training sets used to train a genomic classifier.

FIG. 26 shows a computer control system that may be programmed or otherwise configured to implement methods provided herein.

FIG. 27 shows challenges and solutions in machine learning applications.

FIG. 28 shows an analysis pipeline in the development and evaluation of a molecular genomic classifier to predict usual interstitial pneumonia (UIP) pattern in ILD patients.

FIG. 29 shows gene selection using DESeq2 and a classifier using a volcano plot to show 151 genes selected by DESeq2 (adjusted p-value<0.05 and fold change>2) and 190 predictive genes in a classifier, with 32 common between two sets of genes.

FIG. 30 shows gene selection using DESeq2 and a classifier using a principal component analysis (PCA) plot of all transbronchial biopsies (TBB) samples using only DESeq2 selected genes showing that these genes may not be sufficient to separate UIP samples (circle) from non-UIP samples (cross).

FIG. 31 shows gene selection using DESeq2 and a classifier using a PCA plot of all TBB samples using classifier genes illustrating that TBB samples can be classified into UIP (circle) and non-UIP (cross) samples using these genes.

FIG. 32 shows a comparison between in silico and in vitro mixing within a patient. FIG. 32 shows a scatterplot of in silico and in vitro mixing comparison scored by an ensemble classifier with an R-squared value of 0.99.

FIG. 33 shows a comparison between in silico and in vitro mixing within a patient. FIG. 33 shows a scatterplot of in silico and in vitro mixing comparison scored by a penalized logistic regression classifier with an R-squared value of 0.98.

FIG. 34 shows classification scores of Ensemble Model. Different gray coloring distinguishes samples with histopathology UIP, non-UIP, and non-diagnostic. Circle, up-pointing triangle, square and down-pointing triangle indicate in silico mixed sample, upper, middle and lower lobe samples respectively.

FIG. 35 shows classification scores of Penalized Logistic Regression Model from leave one patient out cross validation. Different gray coloring distinguishes samples with histopathology UIP, non-UIP, and non-diagnostic. Circle, up-pointing triangle, square and down-pointing triangle indicate in silico mixed sample, upper, middle and lower lobe samples respectively.

FIG. 36A-B shows receiver operating characteristic (ROC) curves from leave-one patient-out cross validation (LOPO CV) and validation on independent test set (Testing). The asteroid on each ROC curve corresponds to the prospectively defined decision boundary of each proposed model.

FIG. 37 shows classification performance from leave-one patient-out cross validation and validation on independent test set.

FIG. 38 shows a heatmap of correlation matrix showing intra- and inter-patient heterogeneity in 6-representative patient data with multiple samples.

FIG. 39 shows a PCA plot using genes selected by comparing a non-UIP subtype and UIP samples. The first two principal components in PCA of all training samples using significantly differentially expressed genes comparing UIP samples (circle) and respiratory bronchiolitis (RB).

FIG. 40 shows a PCA plot using genes selected by comparing a non-UIP subtype and UIP samples. The first two principal components in PCA of all training samples using significantly differentially expressed genes comparing UIP samples (circle) and bronchiolitis.

FIG. 41 shows a PCA plot using genes selected by comparing a non-UIP subtype and UIP samples. The first two principal components in PCA of all training samples using significantly differentially expressed genes comparing UIP samples (circle) and hypersensitivity pneumonia (HP).

FIG. 42 shows a PCA plot using genes selected by comparing a non-UIP subtype and UIP samples. The first two principal components in PCA of all training samples using significantly differentially expressed genes comparing UIP samples (circle) and non-specific interstitial pneumonia (NSIP).

FIG. 43 shows a PCA plot using genes selected by comparing a non-UIP subtype and UIP samples. The first two principal components in PCA of all training samples using significantly differentially expressed genes comparing UIP samples (circle) and (organizing pneumonia (OP).

FIG. 44 shows a PCA plot using genes selected by comparing a non-UIP subtype and UIP samples. The first two principal components in PCA of all training samples using significantly differentially expressed genes comparing UIP samples (circle) and sarcoidosis.

FIG. 45 shows variability in gene expressions. The darker upper gray dots indicate genes removed from the training classification.

FIG. 46A-B show threshold vs. sensitivity/specificity in in silico mixed samples using the training set in an Ensemble Model (FIG. 46A) and in a penalized logistic regression model (FIG. 46B).

FIG. 47A-C show score variability simulation for the ensemble model. The final threshold of score variability, 0.90, may be defined by specificity (dotted vertical line) in FIG. 47A. The individual threshold of score variability for sensitivity (1.80) and flip-rate (1.15) may be indicated by a dotted vertical line in FIG. 47B and FIG. 47C.

FIG. 48A-C show score variability simulation for the penalized logistic regression model. The final threshold of score variability, 0.48, may be defined by specificity (vertical line) indicated in FIG. 48A. The individual threshold of score variability for sensitivity (0.78) and flip-rate (0.68) are indicated by gray vertical lines in FIG. 48B and FIG. 48C.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “cancer,” as used herein, generally refers to a condition of abnormal cell growth. The cancer may include a solid tumor or circulating cancer cells. The cancer may metastasize. The cancer may be a tissue-specific cancer. The cancer may be a lung cancer. The cancer may be malignant or benign.

The term “lung cancer,” as used herein, generally refers to a cancer or tumor of a lung or lung-associated tissue. For example, a lung cancer may comprise a non-small cell lung cancer, a small cell lung cancer, a lung carcinoid tumor, or any combination thereof. A non-small cell lung cancer may comprise an adenocarcinoma, a squamous cell carcinoma, a large cell carcinoma, or any combination thereof. A lung carcinoid tumor may comprise a bronchial carcinoid. A lung cancer may comprise a cancer of a lung tissue, such as a bronchiole, an epithelial cell, a smooth muscle cell, an alveoli, or any combination thereof. A lung cancer may comprise a cancer of a trachea, a bronchius, a bronchiole, a terminal bronchiole, or any combination thereof. A lung cancer may comprise a cancer of a basal cell, a goblet cell, a ciliated cell, a neuroendocrine cell, a fibroblast cell, a macrophage cell, a Clara cell, or any combination thereof.

The term “disease or condition,” as used herein, generally refers to an abnormal or pathological condition. A disease or condition may be a lung disease or lung condition. A lung disease or condition may include a lung cancer, interstitial lung disease (ILD), chronic obstructive pulmonary disease (COPD), chronic bronchitis, cystic fibrosis, asthma, emphysema, pneumonia, tuberculosis, pulmonary edema, acute respiratory distress syndrome, or pneumoconiosis. Types of ILD may include idiopathic pulmonary fibrosis, non-specific interstitial pneumonia, desquamative interstitial pneumonia, respiratory bronchiolitis, acute interstitial pneumonia, lymphoid interstitial pneumonia, or cryptogenic organizing pneumonia.

The term “interstitial lung disease” (ILD), as used herein, generally refers to a disease of the interstitial lung tissue. An ILD may comprise an interstitial pneumonia, an idiopathic pulmonary fibrosis, a nonspecific interstitial pneumonitis, a hypersensitivity pneumonitis, a crytogenic organizing pneumonia (COP), an acute interstitial pneumonitis, a desquamative interstitial pneumonitis; a sarcoidosis, an asbestosis, or any combination thereof.

Low-dose computerized tomography (CT) scan (LDCT) generally refers to an imaging procedure that reduces radiation exposure to a subject. For example, a radiation exposure from a LDCT may be less than about 1.5 millisievert (mSv). A radiation exposure from a LDCT may be less than about: 5 mSv, 4 mSv, 3 mSv, 2 mSv, 1 mSv, 0.5 mSv, 0.1 mSv or less. A radiation exposure from a LDCT may be from about 1.0 mSv to about 2.0 mSv. A radiation exposure from an LDCT may be from about 0.5 mSv to about 1.5 mSv. A radiation exposure from an LDCT may be from about 1.0 mSv to about 4.0 mSv. A radiation exposure from an LDCT may be from about 1.0 mSv to about 3.0 mSv. A tube current setting for a LDCT may be less than about: 40 milliampere*seconds (mAs), 35 mAs, 30 mAs, 25 mAs, 20 mAs, 15 mAs, 10 mAs, 5 mAs, 1 mAs or less and still yield sufficient image quality. A tube current setting for a LDCT may be from about 20 mAs to about 40 mAs. A tube current setting from a LDCT may be from about 20 mAs to about 50 mAs. A tube current setting from a LDCT may be from about 20 mAs to about 80 mAs. A tube current setting from a LDCT may be from about 20 mAs to about 100 mAs.

A radiation exposure from a median dose CT scan may be greater than or equal to about 1 mSv, 5 mSv, 6 mSv, 7 mSv, 8 mSv, 9 mSv, 10 mSv, 15 mSv or more. A radiation exposure from a median dose CT scan may be about 8 mSv. A radiation exposure from a median dose CT scan may be from about 7 mSv to about 10 mSv. A radiation exposure from a median dose CT scan may be from about 1 mSv to about 10 mSv. A radiation exposure from a median dose CT scan may be from about 5 mSv to about 10 mSv. A radiation exposure from a median dose CT scan may be from about 1 mSv to about 5 mSv. A tube current setting for a median dose CT scan may be greater than or equal to about: 100 mAs, 125 mAs, 150 mAs, 175 mAs, 200 mAs, 225 mAs, 250 mAs, 300 mAs, 350 mAs, 400 mAs, 500 mAs or more. A tube current setting for a median dose CT scan may be from about 200 mAs to about 250 mAs. A tube current setting for a median dose CT scan may be from about 150 mAs to about 250 mAs. A tube current setting for a median dose CT scan may be from about 100 mAs to about 300 mAs. A tube current setting for a median dose CT scan may be from about 100 mAs to about 200 mAs. A tube current setting for a median dose CT scan may be from about 150 mAs to about 300 mAs. A tube current setting for a median dose CT scan may be from about 150 mAs to about 400 mAs.

The term “homology,” as used herein, generally refers to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences is a function of the number of identical positions shared by the sequences (i.e., % homology=# of identical positions/total # of positions×100). For example, if a position in the first sequence is occupied by the same nucleotide as the corresponding position in the second sequence, then the molecules are identical at that position. The percent homology between the two sequences is a function of the number of identical positions shared by the sequences, taking into account the number of gaps, and the length of each gap, which need to be introduced for optimal alignment of the two sequences. In some embodiments, the length of a sequence aligned for comparison purposes is at least about 30%, at least about 40%, at least about 50%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 95%, of the length of the reference sequence. In some cases, a sequence homology may be from about 70% to 100%. In some cases, a sequence homology may be from about 80% to 100%. In some cases, a sequence homology may be from about 90% to 100%. In some cases, a sequence homology may be from about 95% to 100%. In some cases, a sequence homology may be from about 70% to 99%. In some cases, a sequence homology may be from about 80% to 99%. In some cases, a sequence homology may be from about 90% to 99%. In some cases, a sequence homology may be from about 95% to 99%. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90-5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “fragment,” as used herein, generally refers to a portion of a sequence, such as a subset that may be shorter than a full length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, or 50 amino acids in length. A fragment may be less than about: 20, 30, 40, or 50 nucleotides in length. A fragment may be from about 10 amino acids to about 50 amino acids in length. A fragment may be from about 10 amino acids to about 40 amino acids in length. A fragment may be from about 10 amino acids to about 30 amino acids in length. A fragment may be from about 10 amino acids to about 20 amino acids in length. A fragment may be from about 20 amino acids to about 50 amino acids in length. A fragment may be from about 30 amino acids to about 50 amino acids in length. A fragment may be from about 40 amino acids to about 50 amino acids in length. A fragment may be from about 10 nucleotides to about 50 nucleotides in length. A fragment may be from about 10 nucleotides to about 40 nucleotides in length. A fragment may be from about 10 nucleotides to about 30 nucleotides in length. A fragment may be from about 10 nucleotides to about 20 nucleotides in length. A fragment may be from about 20 nucleotides to about 50 nucleotides in length. A fragment may be from about 30 nucleotides to about 50 nucleotides in length. A fragment may be from about 40 nucleotides to about 50 nucleotides in length.

The term “subject,” as used herein, generally refers to any individual that has, may have, or may be suspected of having a disease condition (e.g., lung disease). The subject may be an animal. The animal can be a mammal, such as a human, non-human primate, a rodent such as a mouse or rat, a dog, a cat, pig, sheep, or rabbit. Animals can be fish, reptiles, or others. Animals can be neonatal, infant, adolescent, or adult animals. The subject may be a living organism. The subject may be a human. Humans can be greater than or equal to 1, 2, 5, 10, 20, 30, 40, 50, 60, 65, 70, 75, 80 or more years of age. A human may be from about 18 to about 90 years of age. A human may be from about 18 to about 30 years of age. A human may be from about 30 to about 50 years of age. A human may be from about 50 to about 90 years of age. The subject may have one or more risk factors of a condition and be asymptomatic. The subject may be asymptomatic of a condition. The subject may have one or more risk factors for a condition. The subject may be symptomatic for a condition. The subject may be symptomatic for a condition and have one or more risk factors of the condition. The subject may have or be suspected of having a disease, such as a cancer or a tumor. The subject may be a patient being treated for a disease, such as a cancer patient, a tumor patient, or a cancer and tumor patient. The subject may be predisposed to a risk of developing a disease such as a cancer or a tumor. The subject may be in remission from a disease, such as a cancer or a tumor. The subject may not have a cancer, may not have a tumor, or may not have a cancer or a tumor. The subject may be healthy.

The term “tissue sample,” as used herein, generally refers to any tissue sample of a subject. A tissue sample may comprise cells obtained from a portion of an airway, such as epithelial cells obtained from a portion of an airway. A tissue sample may be a nasal tissue, a bronchial tissue, a lung tissue, an esophagus tissue, a larynx tissue, an oral tissue or any combination thereof. A tissue sample may be a sample suspected or confirmed of having a disease or condition such as a cancer or a tumor. A tissue sample may be a sample removed from a subject, such as a tissue brushing, a swabbing, a tissue biopsy, an excised tissue, a fine needle aspirate, a tissue washing, a cytology specimen, a bronchoscopy, or any combination thereof. A tissue sample may be an ambiguous or suspicious sample, such as a sample obtained by fine needle aspiration, a bronchoscopy, or other small volume sample collection method. A tissue sample may be an intact region of a patient's body receiving cancer therapy, such as radiation. A tissue sample may be a tumor in a patient's body. A tissue sample may comprise cancerous cells, tumor cells, non-cancerous cells, or a combination thereof. A tissue may comprise invasive cells, non-invasive cells, or a combination thereof. A tissue sample may be a nasal tissue, a trachea tissue, a lung tissue, a pharynx tissue, a larynx tissue, a bronchus tissue, a pleura tissue, an alveoli tissue, breast tissue, bladder tissue, kidney tissue, liver tissue, colon tissue, thyroid tissue, cervical tissue, prostate tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “increased risk” in the context of developing or having a lung condition, as used herein, generally refers to an increased risk or probability associated with the occurrence of a lung condition in a subject. An increased risk of developing a lung condition can include a first occurrence of the condition in a subject or can include subsequent occurrences, such as a second, third, fourth, or subsequent occurrence. An increased risk of developing a lung condition can include a) a risk of developing the condition for a first time, b) a risk of relapse or of developing the condition again, c) a risk of developing the condition in the future, d) a risk of being predisposed to developing the condition in the subject's lifetime, or e) a risk of being predisposed to developing the condition as an infant, adolescent, or adult. An increased risk of a lung condition occurrence or recurrence can include a risk of the condition (such as cancer) becoming metastatic. An increased risk of tumor or cancer occurrence or recurrence can include a risk of occurrence of a stage I cancer, a stage II cancer, a stage III cancer, or a stage IV cancer. Risk of tumor or cancer occurrence or recurrence can include a risk for a blood cancer, tissue cancer (e.g., a tumor), or a cancer becoming metastatic to one or more organ sites from other sites.

The term “an effectiveness of a interventive therapy or treatment regime,” as used herein, generally refers to an assessment or determination about whether an interventive therapy or treatment regime has achieved the results it may be intended to achieve. For example, an effectiveness of a treatment regime, such as administration of an anti-cancer drug, may be an assessment of the anti-cancer drug to reduce a tumor or cancer cell invasiveness, to kill cancer or tumor cells, or to eliminate a cancer or tumor in a subject, to reverse the progression of the disease, or to prevent the disease from developing. A treatment regime may include a surgery (i.e., surgical resection), a nutrition regime, a physical activity, radiation, chemotherapy, cell transplantation, blood fusion, or others. An interventive therapy may include administering to a subject: a pharmaceutical composition, an exercise regime, a dietary regime, a reduction or omission of one or more risk factors (such as smoking or second hand smoke exposure), or any combination thereof.

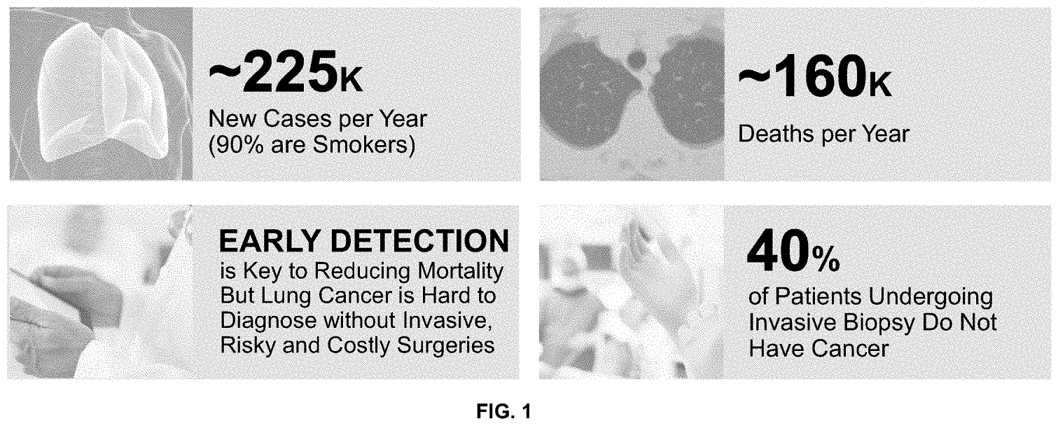

As shown in FIG. 1, greater than about 225,000 new cases of lung cancer may be diagnosed per year. About 90% of subjects newly diagnoses with lung cancer may be subjects having a prior history of smoking. Lung cancer causes about 160,000 deaths per year. Developing new methods, systems, and kits, such as those described herein, may improve early detection of lung cancer or an increased risk of developing lung cancer, wherein early detection may be a key improvement for reducing overall mortality. Further, current clinical standards of care make it difficult to accurately diagnose lung cancer without the need for invasive, high-risk, costly invasive procedures, such as surgery or lung biopsy. Approximately 40% of subjects undergoing an invasive lung biopsy as part of a current clinical standard of care do not have cancer. Therefore, new methods, systems, and kits, such as those described herein, may also reduce the number of unnecessary invasive procedures (carrying associated risks and extra costs) while improving early detection and highly accurate diagnosis of lung cancer.

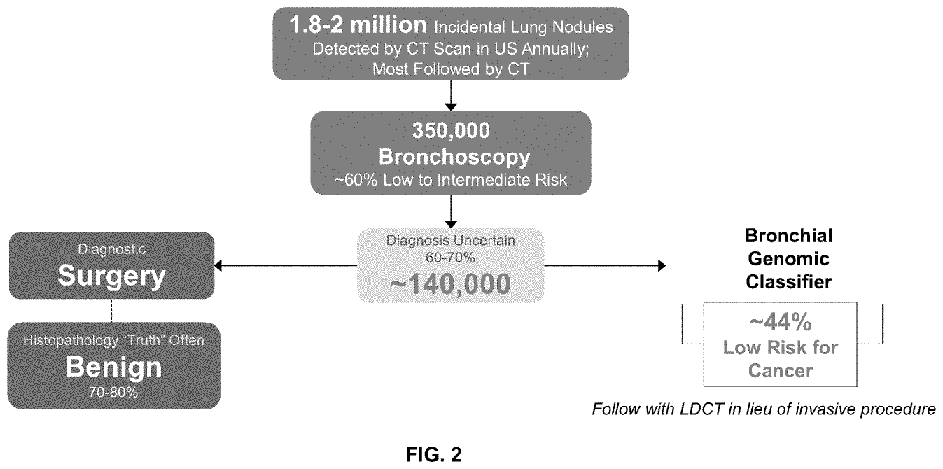

As shown in FIG. 2, integrating genomic classifiers at different decision points within current clinical standards of care can reduce the number of unnecessary invasive procedures and identify subjects having low risk for lung cancer. For example, about 1.8 million to 2 million cases of incidental lung nodules may be detected by imaging scans in the US annually. The current clinical standard of care dictates these subjects, having nodules detected by imaging scan, then receive an invasive bronchoscopy to further evaluate whether lung nodules may be indicative of a presence of lung cancer. About 140,000 subjects (or about: 60-70% of the 350,000 subjects having a bronchoscopy) may receive an ambiguous or suspicious result. Current clinical standard of care dictates that bronchoscopies having an ambiguous or suspicious result, then receive a diagnostic surgery to determine a histopathological truth. However, about 70-80% of those subjects having an ambiguous or suspicious result may have lung tissue that may be histopathologically benign. Therefore, new methods, systems, and kits, such as those described herein, can improve the current clinical standard of care such that an ambiguous or suspicious result will be followed by analysis on one or more genomic classifiers to identify subjects having a low risk of lung cancer from those subjects having an increased risk or high risk of lung cancer. And, those subjects having an increased risk or high risk of lung cancer will be subjected to the invasive diagnostic surgery—thereby avoiding an unnecessary invasive procedure on a low-risk population.

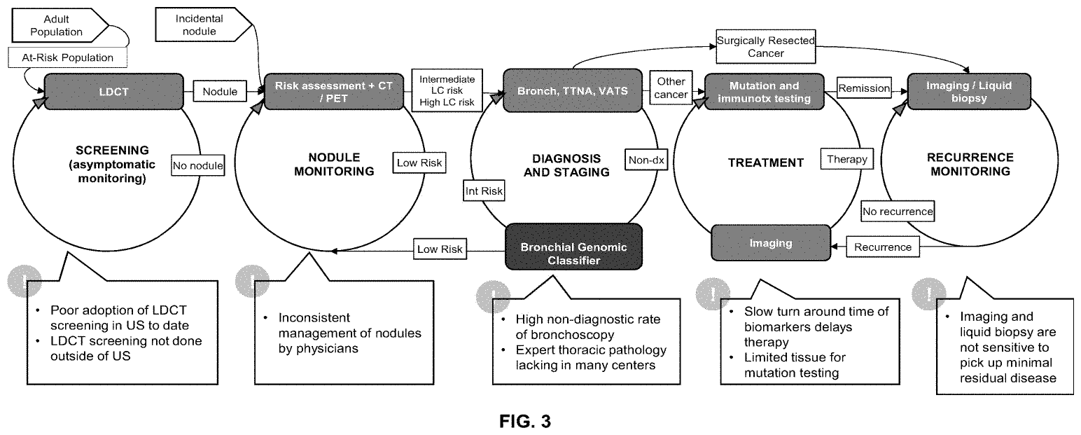

FIG. 3 shows a current clinical standard of care with the addition/improvement of a bronchial genomic classifier as described herein. From a generic adult population, those individuals identified as at-risk for lung cancer may receive an imaging scan, such as a low dose CT scan. If no nodules may be identified, another imaging scan may be obtained at a later time point. If a nodule may be identified, a subject may receive a risk assessment, a CT scan, a PET scan, magnetic resonance imaging (MM) scan, an X-ray, or any combination thereof. Currently, there is poor adoption of low dose CT scanning in the United States. If a risk assessment, a CT scan, a PET scan, an MRI scan, an X-ray, or any combination thereof identifies the subject as having an low risk of lung cancer, then another risk assessment, another CT scan, another PET scan, another MRI scan, another X-ray, or any combination thereof may be performed at a later time point. If a risk assessment, a CT scan, a PET scan, an Mill scan, an X-ray, or any combination thereof identifies the subject as having an intermediate or high risk of lung cancer, a subject may receive a bronchoscopy, a transthoracic needle aspiration (TTNA), a video-assisted thoracic-scopic surgery (VATS), any method to obtain an airway tissue sample, or any combination thereof. If the airway sample obtained is identified as ambiguous or suspicious, a bronchial genomic classifier may be run to identify the risk of lung cancer. If the bronchial genomic classifier identifies the sample as a low risk, then another risk assessment, another CT scan, another PET scan, another MM scan, another X-ray, or any combination thereof may be performed. If the bronchial genomic classifier identifies a sample as intermediate risk, then another bronchoscopy, another transthoracic needle aspiration (TTNA), another video-assisted thoracic-scopic surgery (VATS), another method to obtain an airway tissue sample, or any combination thereof may be performed. A bronchoscopy sample may ambiguous or suspicious. A high percentage of bronchoscopy samples may be ambiguous or suspicious. Therefore, adding a bronchial genomic classifier to the current clinical standard of care may significantly reduce the number of ambiguous or suspicious results. If a subject is identified as having a lung cancer, the subject may treated for the lung cancer and may be monitored for recurrence of lung cancer by imaging, liquid biopsy, or a combination thereof. However, these current methods of imaging and liquid biopsy to identify disease recurrence suffer from low sensitivity and minimal ability to identify residual disease.

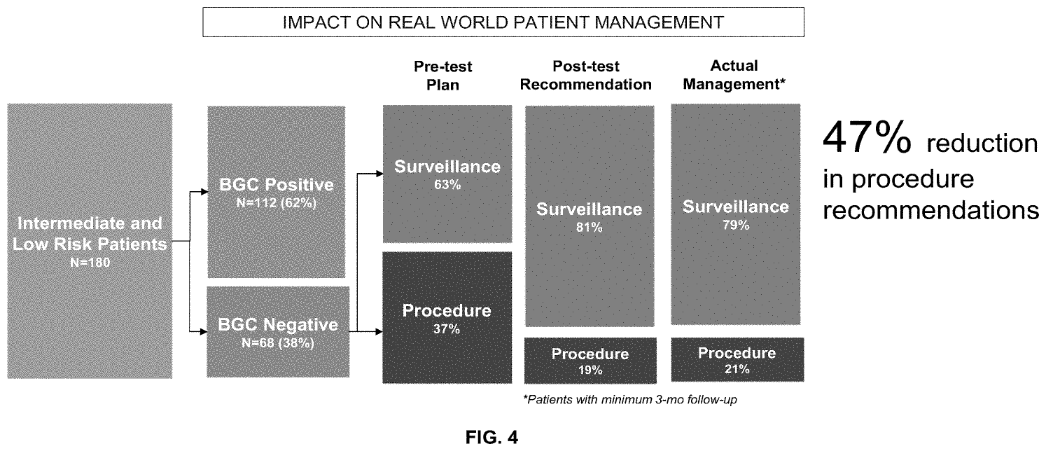

As shown in FIG. 4, addition of a bronchial genomic classifier to the clinical standard of care of lung cancer may significantly improve subject management and may have of positive impact. For example, prior to the addition of a bronchial genomic classifier, about 37% or more of intermediate to low risk subjects may be subjected to an invasive procedure. In contrast, by the addition of a bronchial genomic classifier to the clinical standard of care, there may be a reduction of about 47% or more in the number of invasive procedures performed on intermediate to low risk subjects.

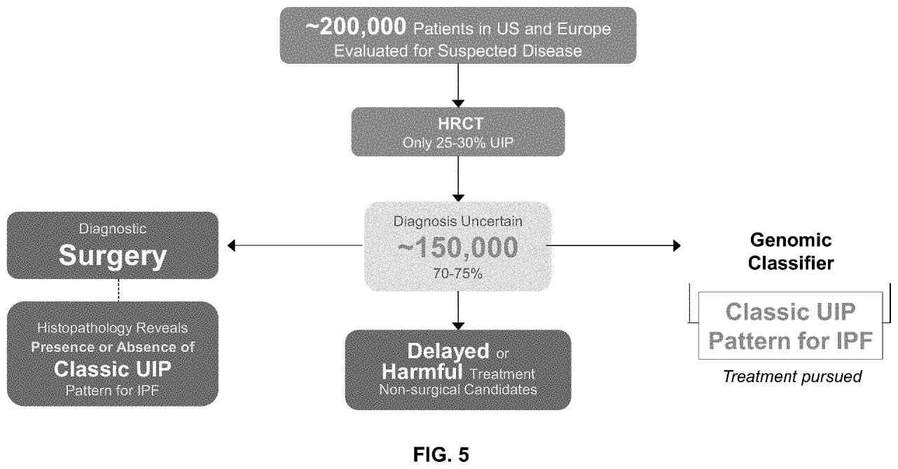

As shown in FIG. 5, addition of a genomic classifier to the clinical standard of care of idiopathic pulmonary fibrosis (IPF) may significantly reduce the number of unnecessary invasive procedures. For example, about 200,000 subjects in the US and Europe may be evaluated for a suspected presence of IPF and may receive a diagnostic high-resolution computed tomography (HRCT). Of those 200,000 subjects, about 150,000 subjects (or 70-75%) may receive an ambiguous or suspicious result from the HRCT. Those subjects having an ambiguous or suspicious result, may receive a diagnostic surgery to identify a histopathological truth (a presence or absence of IPF). However, implementation of a genomic classifier as described herein, may identify a presence or an absence of a classic interstitial pneumonia pattern (UIP) (a pattern for IPF). In the case of an identification of a presence of classic UIP, a subject may then receive a diagnostic surgery or treatment. In the case of an identification of an absence of classic UIP, a subject may not receive an invasive procedure.

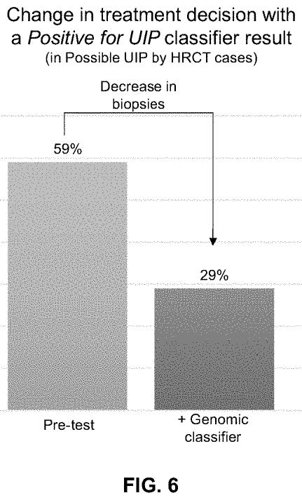

FIG. 6 shows a graph of percent decrease in the number of biopsies and highlights the clinical utility of employing a genomic classifier in differentiating UIP from other ILD pathologies. For example, introduction of a genomic classifier may have a strong clinical impact on improving management approaches for ILD. A significant decrease in the number of invasive biopsies may be observed by the inclusion of a genomic classifier in differentiating UIP from other ILD pathologies.

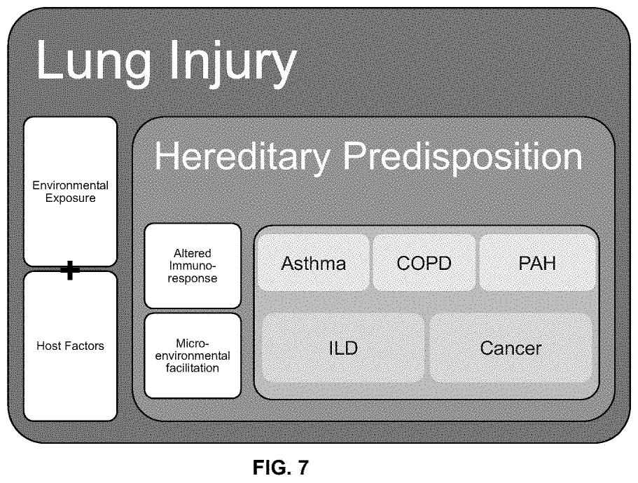

As shown in FIG. 7, the etiologic field of injury may share common pathways. For example, etiologic exposures and chronic airway injury may modify a tissue microenvironment, such as an airway epithelial environment. An altered microenvironment may result in one or more molecular aberrations and activation of one or more repair pathways. Phenotype may be determined by intrinsic host response to an injury. COPD, ILD, asthma or any combination thereof may reflect a host response that may increase risk for a lung cancer. Biomarker analysis from airway epithelium may represent significant opportunities to identify the continuum of change.

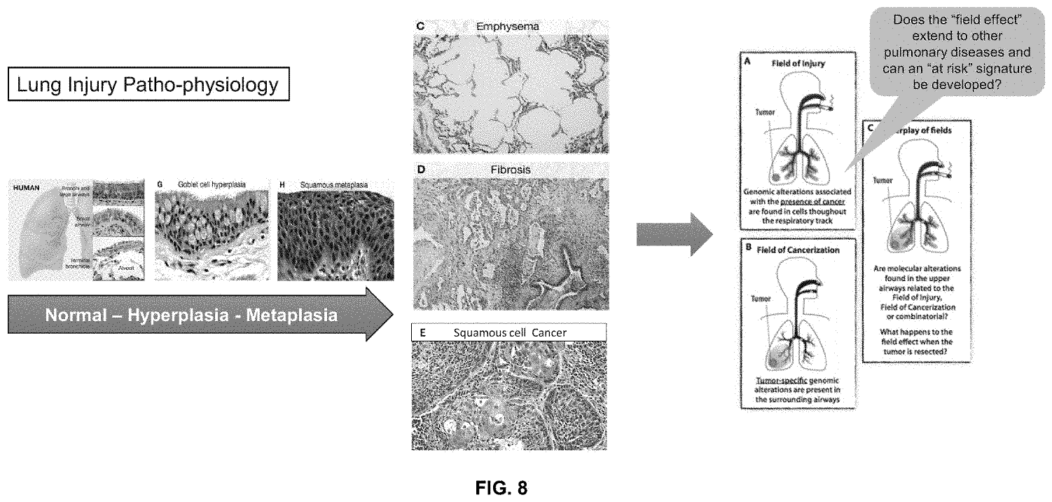

As shown in FIG. 8, there may be more than one field, such as a field of cancerization and a field of injury. A field of injury may include genomic alterations associated with a presence of a lung cancer that may be found in cells throughout the respiratory track. A field of cancerization may include tumor-specific genomic alterations that may be present in the surrounding airways, such as proximal a tumor source. There may be interplay between a field of injury and a field of cancerization. For example, molecular alternations found in the upper airway may or may not be related to the field of injury, the field of cancerization, or a combination thereof. An at-risk molecular signature may be implemented for any lung condition, such as a lung cancer, ILD, COPD, asthma, or others.

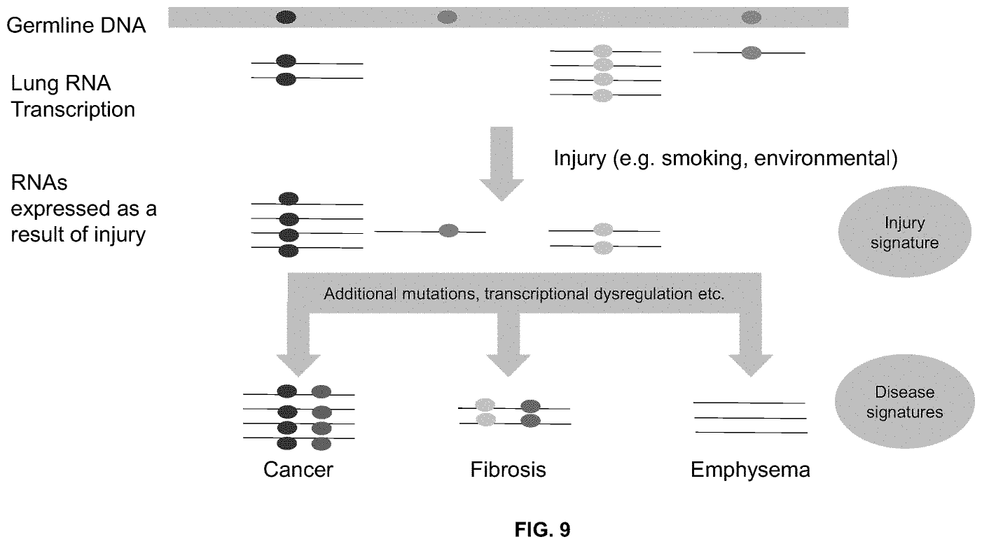

FIG. 9 shows a molecular view of the field of injury and field of cancerization concepts. Injury may include smoking or environmental exposures. Injury signatures (such as altered RNA expression) and disease signatures (such as additional mutations, transcriptional dysregulation, and others) may be outlined for lung conditions such as cancer, fibrosis, and emphysema.

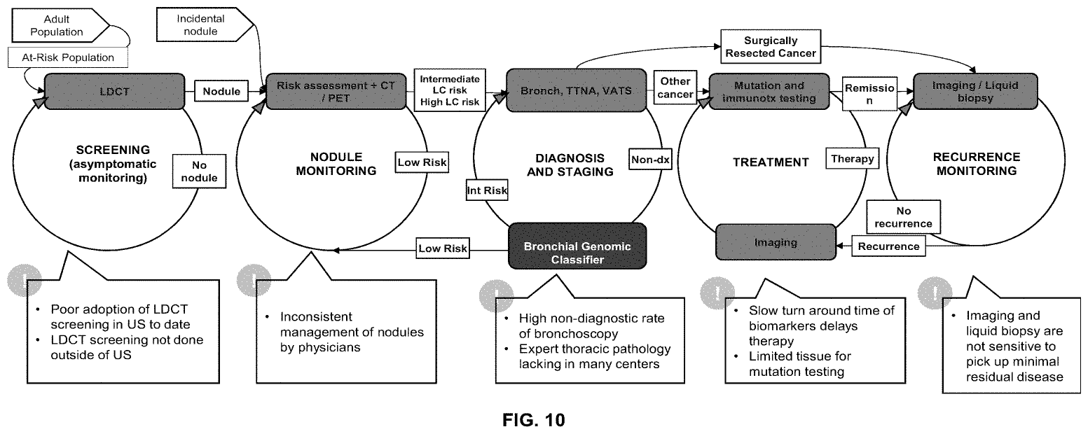

FIG. 10 shows a similar pathway to FIG. 3 showing the current state of clinical decisions improved by the addition of a single bronchial genomic classifier. However, the current state of clinical care may benefit from the addition of other genomic classifiers at other decision points within the clinical care pathway.

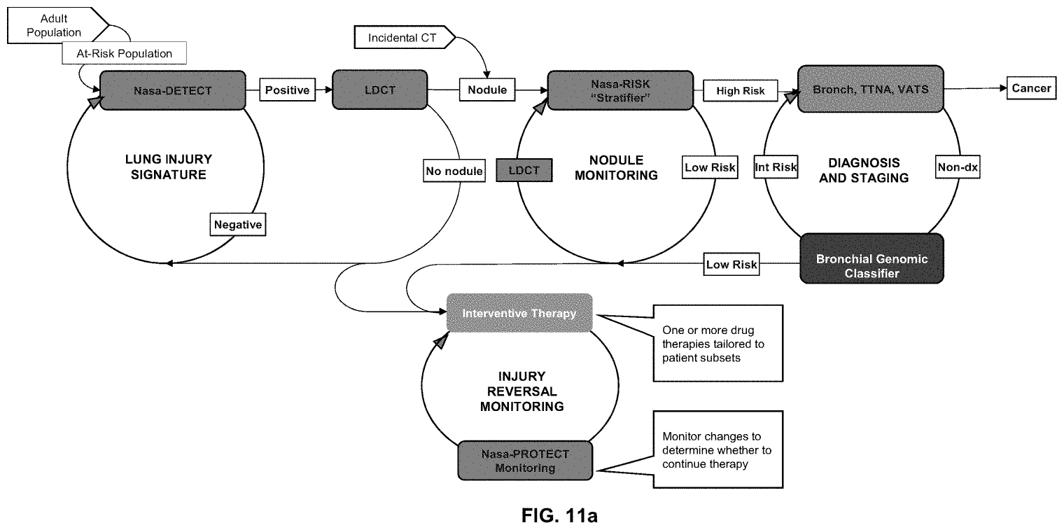

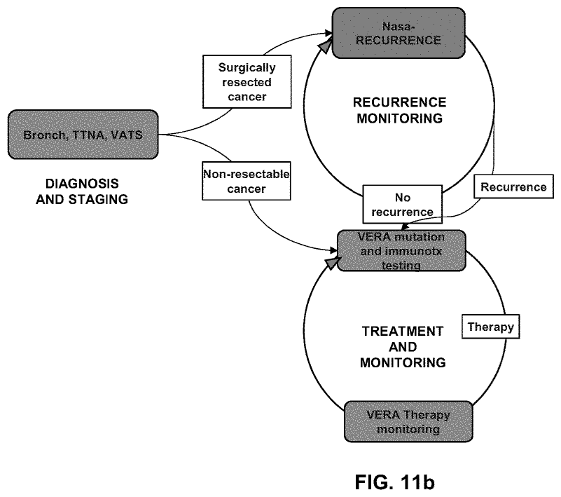

FIG. 11a and FIG. 11b show addition of various genomic classifiers at specific decision points within the current clinical standard of care that improve early detection and minimize unnecessary invasive procedures. For example, an at-risk population may be identified within a generic population. An at-risk population may include subjects having an increased risk of developing or having a lung condition (such as lung cancer). An at-risk population may be identified by identifying a presence of one or more risk factors associated with the lung condition. Subjects may be given a questionnaire that may assess the presence of the one or more risk factors. Subjects may be prompted by a medical professional to provide answers to questions that may assess the presence of the one or more risk factors. A sample (such as a non-invasive sample, such as a nasal brushing) may be obtained from subjects that may be identified as at-risk for the lung condition. Data obtained from the sample (such as for example expression levels or sequence variant data) may be input to a genomic classifier (such as a Nasa-DETECT classifier). The genomic classifier may identify the sample as positive or negative. A subject receiving a positive result may receive an imaging scan (such as a low-dose CT scan) to scan for lung nodules. A subject receiving a negative result may have another sample obtained at a later time point, the data from which may be input to the genomic classifier.

Subjects having a confirmed presence of a lung nodule based on an imaging scan (such as a low-dose CT scan), may have a sample obtained. Data from the sample (such as expression levels or sequence variant data) may be input to a genomic classifier (such as a Nasa-RISK classifier). The genomic classifier may identify the sample as high risk or low risk for a lung condition (such as lung cancer). A subject receiving a high risk result from the classifier may receive an invasive procedure (such as a bronchoscopy, a TTNA, or a VATS) to confirm a presence or an absence of the lung condition. A subject receiving a low risk result from the classifier may receive another imaging scan to scan for the presence of a nodule followed by inputting data from another sample into the genomic classifier at a later time point.

Subjects having a low risk of a lung condition as identified by a genomic classifier (such as the Nasa-RISK Stratifier classifier or the Bronchial Genomic Classifier) may receive an interventive therapy to slow or reversal disease progression or prevent occurrence of a lung condition. A sample from a subject may be obtained following at least completion of a portion of the interventive therapy. Data from the sample (such as expression levels or sequence variant data) may be input to a genomic classifier (such as a Nasa-PROTECT Monitoring classifier). The genomic classifier may identify the efficacy of the interventive therapy, a subject compliance, a disease reversal or lung condition prevention, or a combination thereof.

Subjects having a curative treatment such as a surgically resected cancer or a therapy regime (such as administration of a pharmaceutical composition), may have a sample obtained following the curative treatment. Data from the sample (such as expression levels or sequence variant data) may be input to a genomic classifier (such as a Nasa-RECURRENCE classifier). The genomic classifier may provide early detection of a lung condition recurrence.

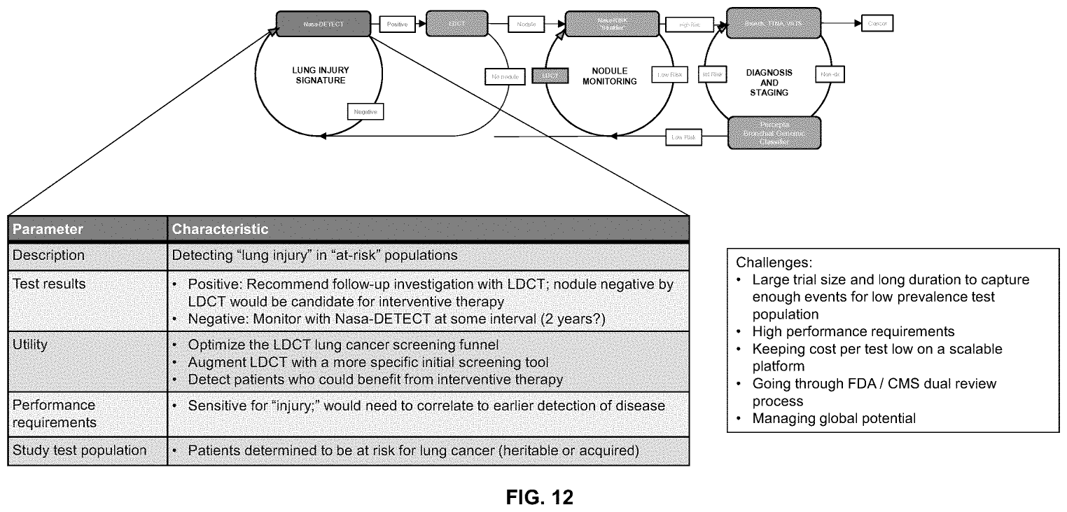

FIG. 12 shows characteristics of a Nasa-DETECT classifier. This classifier may detect lung injury in at-risk populations. This classifier may (i) optimize an imaging screening funnel; (ii) may augment an imaging scan with a more specific initial screening tool; (iii) may enhance early detection of subjects whom may benefit from interventive therapy; or (iv) any combination thereof. Subjects evaluated by this classifier may be previously determined to be at risk for lung cancer. A positive result from this classifier may include a recommendation for a follow-up investigation with an imaging scan (such as a LDCT) and an absence of nodules by the LDCT may indicate the subject as a candidate for interventive therapy. A negative result from this classifier may include monitoring again with this classifier at a later time point.

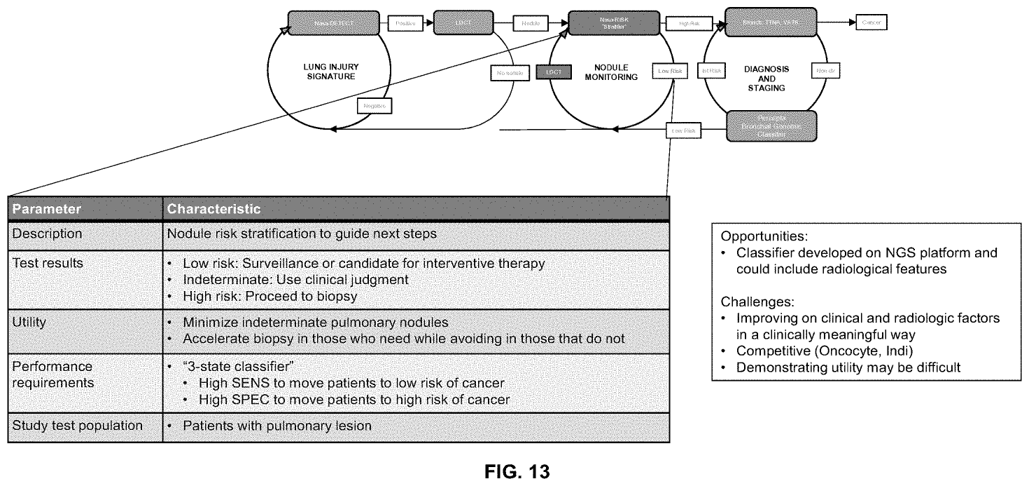

FIG. 13 shows characteristics of a Nasa-RISK Stratifier classifier. This classifier may stratify nodule risk. This classifier may minimize the number of indeterminate pulmonary nodules. This classifier may accelerate biopsy in those subjects who may need a biopsy while avoiding an invasive biopsy in those subjects that do not need one. Subjects evaluated by this classifier may include subjects having an identified pulmonary lesion. A low risk result from this classifier may include surveillance or an indication of the subject as a candidate for an interventive therapy. An intermediate result from this classifier may include a use of clinical judgement. A high risk result from this classifier may include a subject receiving a biopsy. This classifier may be developed on a Next-Generation Sequencing (NGS) platform. This classifier may include sequencing information, radiological features, or a combination thereof.

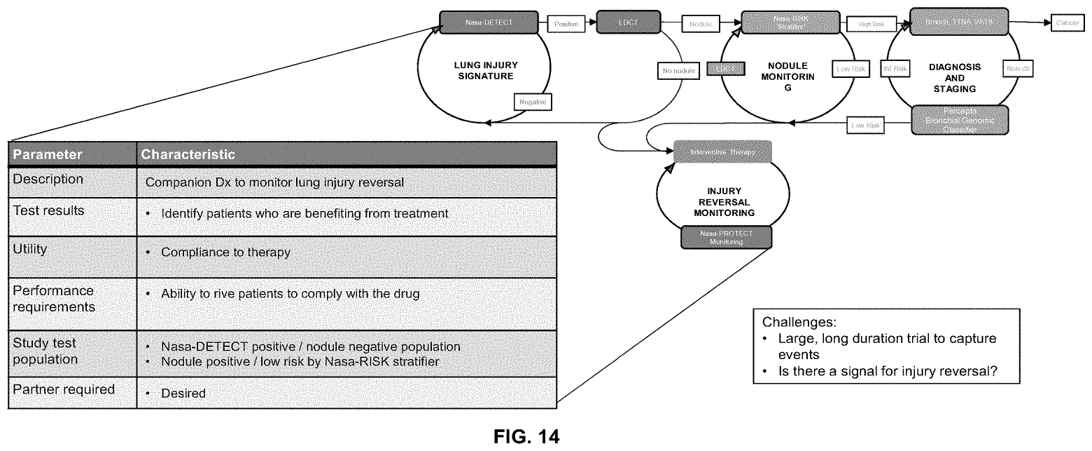

FIG. 14 shows characteristics of a Nasa-PROTECT classifier. This classifier may be a companion diagnostic to monitor lung injury reversal. This classifier may identify subject compliance with a given treatment or therapy. This classifier may identify subjects that may be benefiting from a recommended treatment or therapy. Subjects evaluated by this classifier may include Nasa-DETECT positive and nodule negative subject populations. Subjects evaluated by this classifier may include nodule positive and low risk by Nasa-RISK Stratifier classifier.

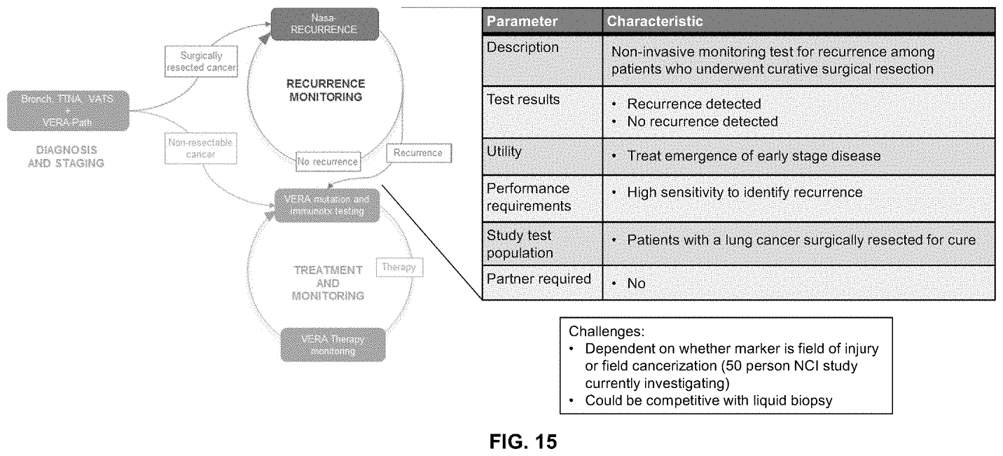

FIG. 15 shows characteristics of Nasa-RECURRENCE classifier. This classifier may be a non-invasive monitoring method to test for recurrence among subject having received a curative surgical resection or curative treatment regime. This classifier may identify emergence or reemergence of early stage disease. This classifier may comprise high sensitivity to identify recurrence. Subjects evaluated by this classifier may include subjects having a lung cancer surgically resected for cure or receiving a curative treatment regime.

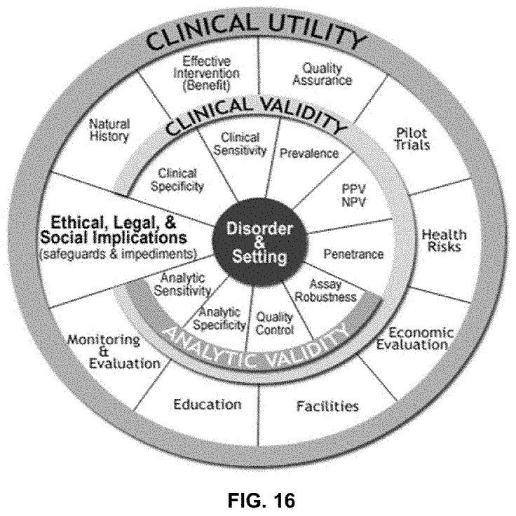

FIG. 16 shows the ACCE evaluation process for genetic testing. The four main criteria in evaluations a genetic test include Analytic validity, Clinical validity, Clinical utility, and Ethical implications.

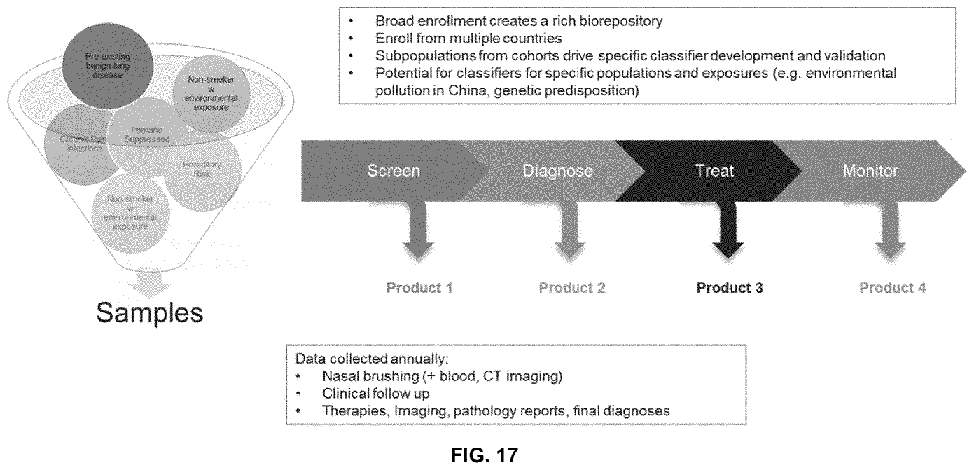

FIG. 17 shows examples of (i) types samples used to train and to validate genomic classifiers and (ii) types of samples input into a genomic classifier for identification. Samples may include samples obtained from: a subject having a pre-existing benign lung disease; a subject having chronic pulmonary infections; a subject having a suppressed immune system; a subject having an increased hereditary risk of developing a lung condition; a non-smoker having environmental exposure; or any combination thereof. Samples may be obtained from a plurality of different countries. Subpopulations from cohorts may drive specific classifier development and validation. Classifiers may be developed for specific population, types of exposures, or combinations thereof. For example, classifiers may be developed for environmental pollution in China or for a genetic predisposition to a lung condition. A genomic classifier may be developed to screen for a lung condition, to diagnose a lung condition, to evaluate a treatment for a lung condition, to monitor a subject's condition, or any combination thereof. Samples may be collected annually from a subject. Samples obtained annually may include nasal brushing, a blood sample, an imaging scan, or combinations thereof.

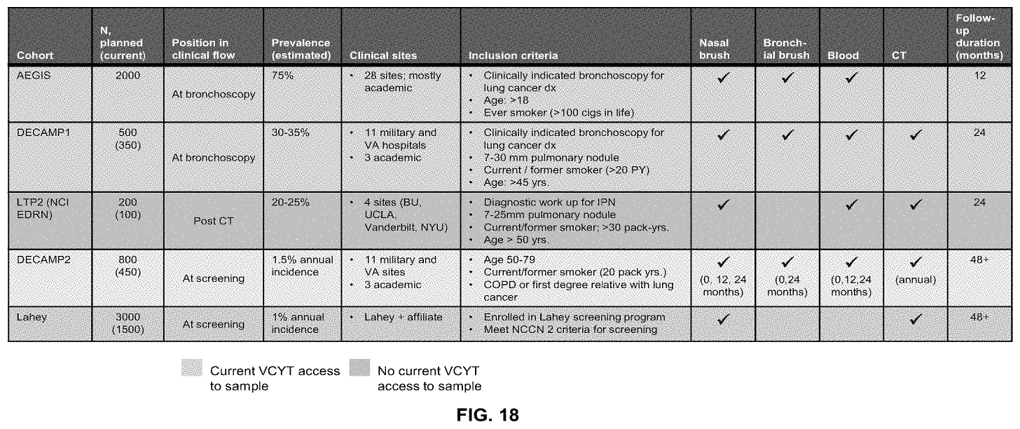

FIG. 18 shows cohorts with nasal or bronchial brushing samples. Each cohort may be identified (AEGIS, DECAMP1, LTP2, DECAMP2, and Lahey). The number of subjects enrolled and the position in the current standard of care may be identified (at bronchoscopy, post imaging scan, or at screening) and indicated for each sample cohort. Inclusion criteria may be indicated, including age of subject and smoking history. Types of samples (nasal brush, bronchial brush, blood, imaging scan) and follow-up duration (12 months, 24 months, 48 months) may also be indicated for each sample cohort.

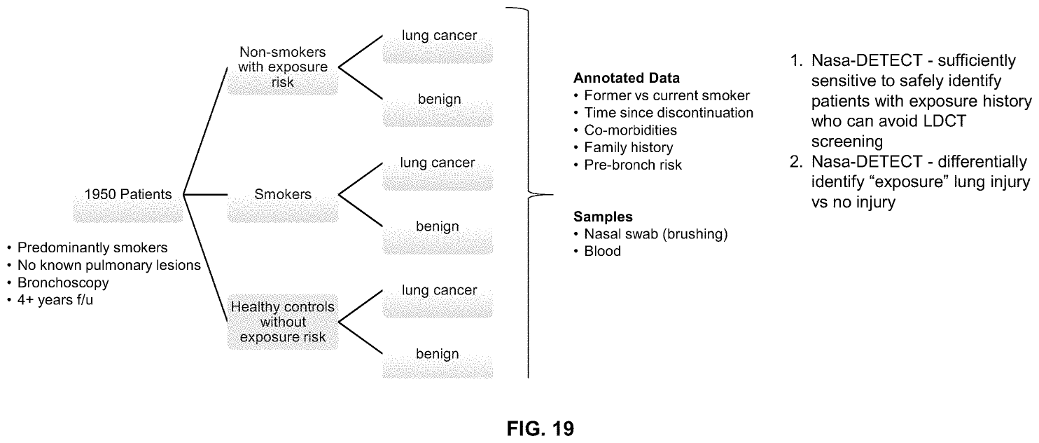

FIG. 19 shows examples of training samples used to train and validate a classifier (such as a Nasa-DETECT classifier). Cohorts DECAMP2 and Lahey may be employed for training of this classifier. Samples may include nasal brushing, blood samples, or a combination thereof. Additional data may be collected from each subject providing a sample including: whether the subject may be a former or current smoker; time since discontinuation of smoking; presence of co-morbidities; a family history of lung conditions; a pre-bronchial risk; or any combination thereof. Training samples used to train and validate a classifier may be greater than about: 100 samples, 200 samples, 300 samples, 400 samples, 500 samples, 600 samples, 700 samples, 800 samples, 900 samples, 1000 samples, 1100 samples, 1200 samples, 1300 samples, 1400 samples, 1500 samples, 1600 samples, 1700 samples, 1800 samples, 1900 samples, 2000 samples, or more (for example 1950 samples obtained from different subjects). In some cases, training samples may comprise from about 100 samples to about 200 samples. In some cases, training samples may comprise from about 100 samples to about 300 samples. In some cases, training samples may comprise from about 100 samples to about 400 samples. In some cases, training samples may comprise from about 100 samples to about 500 samples. In some cases, training samples may comprise from about 100 samples to about 600 samples. In some cases, training samples may comprise from about 100 samples to about 700 samples. In some cases, training samples may comprise from about 100 samples to about 800 samples. In some cases, training samples may comprise from about 100 samples to about 900 samples. In some cases, training samples may comprise from about 100 samples to about 1000 samples. In some cases, training samples may comprise from about 100 samples to about 1500 samples. In some cases, training samples may comprise from about 100 samples to about 2000 samples. In some cases, training samples may comprise from about 100 samples to about 3000 samples. In some cases, training samples may comprise from about 100 samples to about 4000 samples. In some cases, training samples may comprise from about 100 samples to about 5000 samples. Subjects providing a sample may be smokers, non-smokers with exposure risk, or health subjects without a smoking history or exposure risk.

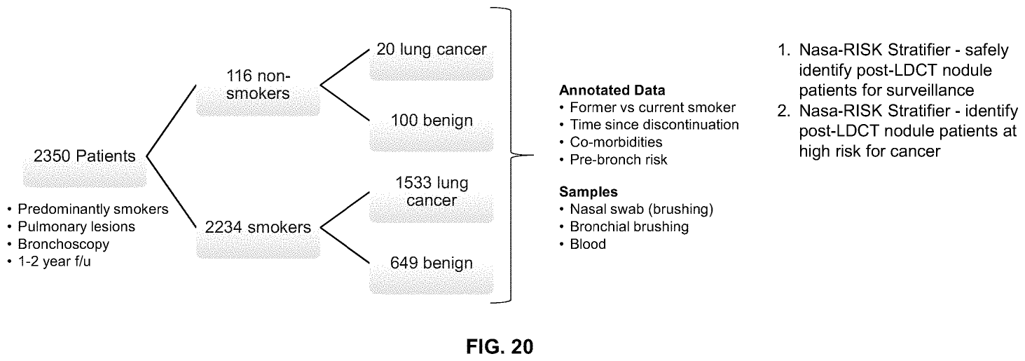

FIG. 20 shows examples of training samples used to train and validate a classifier (such as a Nasa-RISK Stratifier classifier. Cohorts AEGIS and DECAMP1 may be employed for training of this classifier. Samples may include nasal brushing, bronchial brushing, blood sample, or any combination thereof. Additional data may be collected from each subject providing a sample including: whether the subject may be a former or current smoker; time since discontinuation of smoking; presence of co-morbidities; a pre-bronchial risk; or any combination thereof. Training samples used to train and to validate a classifier may be greater than about: 100 samples, 200 samples, 300 samples, 400 samples, 500 samples, 600 samples, 700 samples, 800 samples, 900 samples, 1000 samples, 1100 samples, 1200 samples, 1300 samples, 1400 samples, 1500 samples, 1600 samples, 1700 samples, 1800 samples, 1900 samples, 2000 samples, 2100 samples, 2200 samples, 2300 samples, 2400 samples, 2500 samples, 2600 samples, 2700 samples, 2800 samples 2900 samples, 3000 samples, or more (for example 2350 samples obtained from different subjects). In some cases, training samples may comprise from about 100 samples to about 200 samples. In some cases, training samples may comprise from about 100 samples to about 300 samples. In some cases, training samples may comprise from about 100 samples to about 400 samples. In some cases, training samples may comprise from about 100 samples to about 500 samples. In some cases, training samples may comprise from about 100 samples to about 600 samples. In some cases, training samples may comprise from about 100 samples to about 700 samples. In some cases, training samples may comprise from about 100 samples to about 800 samples. In some cases, training samples may comprise from about 100 samples to about 900 samples. In some cases, training samples may comprise from about 100 samples to about 1000 samples. In some cases, training samples may comprise from about 100 samples to about 1500 samples. In some cases, training samples may comprise from about 100 samples to about 2000 samples. In some cases, training samples may comprise from about 100 samples to about 3000 samples. In some cases, training samples may comprise from about 100 samples to about 4000 samples. In some cases, training samples may comprise from about 100 samples to about 5000 samples. Subjects providing a sample may be smokers or non-smokers.

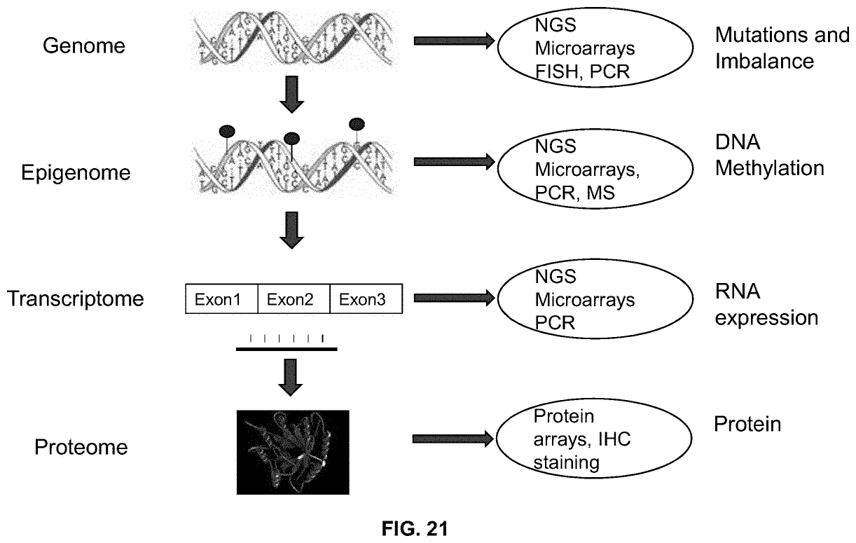

FIG. 21 shows biomarkers and the technology employed to detect their presence or absence. For example, genomic biomarkers (including mutations and imbalance) may be detected by next-generation sequencing (NGS), microarrays, fluorescent in situ hybridization (FISH), polymerase chain reaction (PCR), or any combination thereof. Epigenetic biomarkers (such as DNA methylation, such as 5-hydroxymethylated cytosine, 5-methylated cytosine, 5-carboxymethylated cytosine, or 5-formylated cytosine) may be detected by NGS, microarrays, PCR, mass spectrometry (MS), or any combination thereof. Transcriptomic biomarkers (such as RNA expression levels) may be detected by NGS, microarrays, PCR, or any combination thereof. Proteomic biomarkers (such as a presence of a protein) may be detected by protein arrays, immunohistochemical staining (IHC), or a combination thereof.

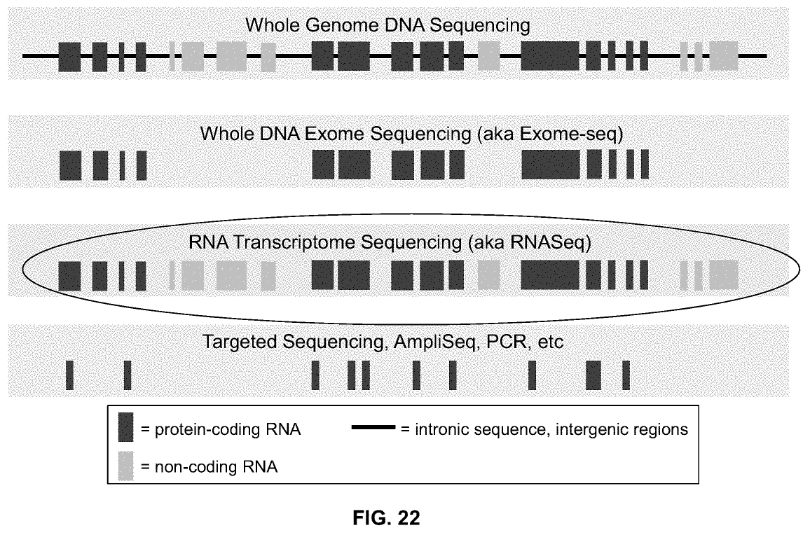

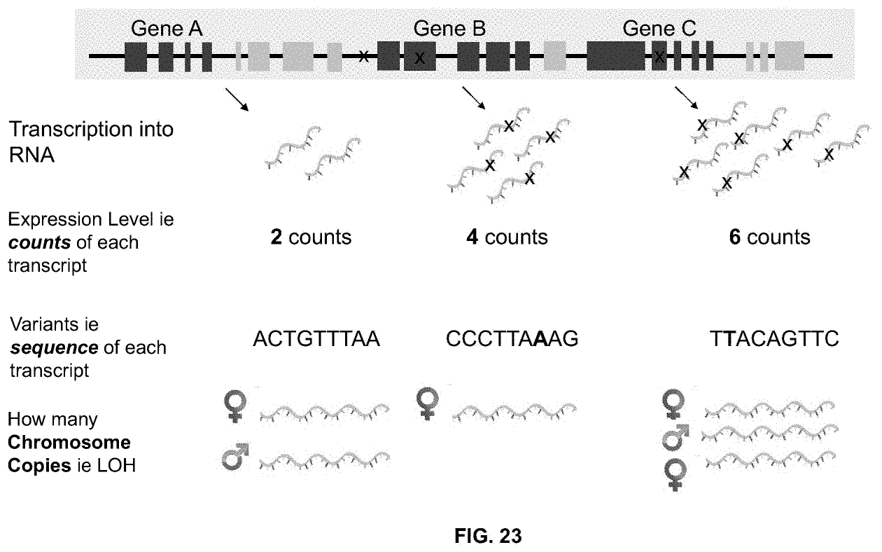

FIG. 22 shows RNA sequencing for a genomic classifier and thyroid FNA analysis of the genomic classifier. FIG. 23 shows an example of RNA sequencing of gene A, gene B, and gene C. Transcription into RNA may be followed by: (i) detecting one or more expression levels (such as counts of each transcript); (ii) detecting one or more variants (such as a sequence of each transcript); (iii) detecting a number of chromosome copies (such as loss of heterozygosity (LOH)); or (iv) any combination thereof.

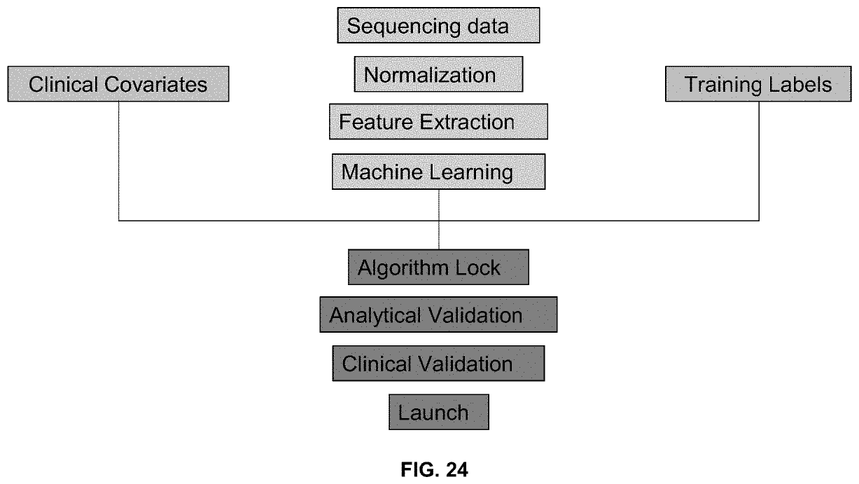

FIG. 24 shows a flow diagram of a trained algorithm as described herein. For example, an algorithm may receive one or more types of sequencing data from a sample. Data received into an algorithm may be normalized. Feature extraction or feature selection may occur along with supervised machine learning. One or more clinical covariates may be added to the algorithm. One or more training labels may be added to the algorithm. One or more locks may be incorporated into the algorithm. Analytical validation may be confirmed. Clinical validation may be confirmed. A genomic classifier may be launched.

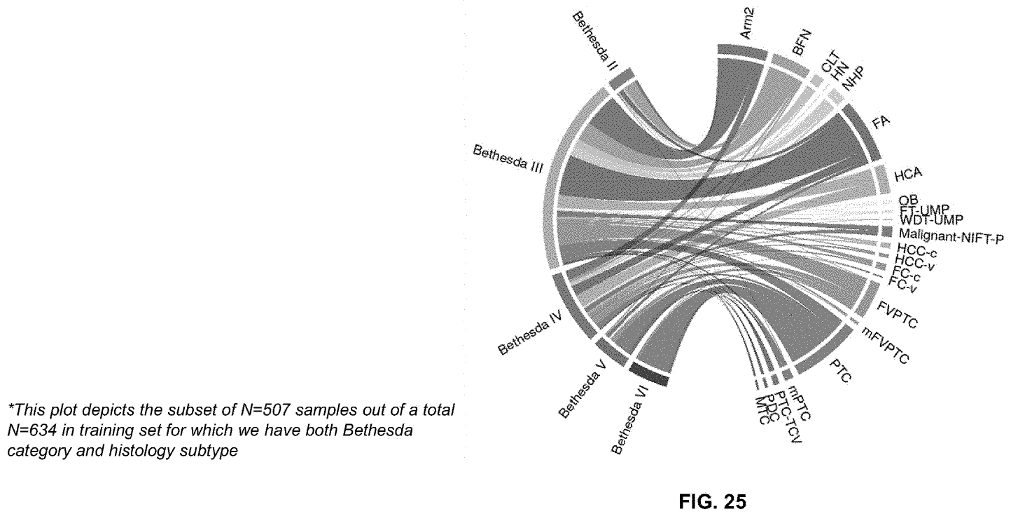

FIG. 25 shows an example of a training set rich in Bethesda cytology and histology subtypes. For example, FIG. 25 shows 507 samples of a total 634 samples in a training set that have both Bethesda cytology and histology subtypes. A training set may span all biological categories.

Accuracy, Specificity and Sensitivity

A method as described herein may (i) determine a presence or an absence of a condition, such as a lung cancer or (ii) classify a tissue as benign or malignant, such methods may provide a specificity of diagnosis that may be greater than about 70%. In some embodiments, the specificity may be at least about: 70%, 75%, 80%, 85%, 86%, 87%, 88%, 89%, 90%, 91%, 92%, 93%, 94%, 95%, 96%, 97%, 98%, 99%, or more. In some cases, the specificity may be from about 70% to about 99%. In some cases, the specificity may be from about 80% to about 99%. In some cases, the specificity may be from about 85% to about 99%. In some cases, the specificity may be from about 90% to about 99%. In some cases, the specificity may be from about 95% to about 99%. In some cases, the specificity may be from about 70% to about 95%. In some cases, the specificity may be from about 80% to about 95%. In some cases, the specificity may be from about 85% to about 95%. In some cases, the specificity may be from about 90% to about 95%. In some cases, the specificity may be from about 70% to 100%. In some cases, the specificity may be from about 80% to 100%. In some cases, the specificity may be from about 85% to 100%. In some cases, the specificity may be from about 90% to 100%. In some cases, the specificity may be from about 90% to 100%.

A method as described herein may (i) determine a presence or an absence of a condition, such as a lung cancer or (ii) classify a tissue as benign or malignant, such methods may provide a sensitivity of diagnosis that may be greater than about 70%. In some embodiments, the sensitivity may be at least about: 70%, 75%, 80%, 85%, 86%, 87%, 88%, 89%, 90%, 91%, 92%, 93%, 94%, 95%, 96%, 97%, 98%, 99%, or more. In some cases, the sensitivity may be from about 70% to about 99%. In some cases, the sensitivity may be from about 80% to about 99%. In some cases, the sensitivity may be from about 85% to about 99%. In some cases, the sensitivity may be from about 90% to about 99%. In some cases, the sensitivity may be from about 95% to about 99%. In some cases, the sensitivity may be from about 70% to about 95%. In some cases, the sensitivity may be from about 80% to about 95%. In some cases, the sensitivity may be from about 85% to about 95%. In some cases, the sensitivity may be from about 90% to about 95%. In some cases, the sensitivity may be from about 70% to 100%. In some cases, the sensitivity may be from about 80% to 100%. In some cases, the sensitivity may be from about 85% to 100%. In some cases, the sensitivity may be from about 90% to 100%. In some cases, the sensitivity may be from about 90% to 100%.

A method as described herein may (i) determine a presence or an absence of a condition, such as a lung cancer or (ii) classify a tissue as benign or malignant, such methods may provide a sensitivity of diagnosis that may be greater than about 70% and a specificity that may be greater than about 70%. The sensitivity may be greater than about 70% and the specificity may be greater than about 80%. The sensitivity may be greater than about 70% and the specificity may be greater than about 90%. The sensitivity may be greater than about 70% and the specificity may be greater than about 95%. The sensitivity may be greater than about 80% and the specificity may be greater than about 70%. The sensitivity may be greater than about 80% and the specificity may be greater than about 80%. The sensitivity may be greater than about 80% and the specificity may be greater than about 90%. The sensitivity may be greater than about 80% and the specificity may be greater than about 95%. The sensitivity may be greater than about 90% and the specificity may be greater than about 70%. The sensitivity may be greater than about 90% and the specificity may be greater than about 80%. The sensitivity may be greater than about 90% and the specificity may be greater than about 90%. The sensitivity may be greater than about 90% and the specificity may be greater than about 95%. The sensitivity may be greater than about 95% and the specificity may be greater than about 70%. The sensitivity may be greater than about 95% and the specificity may be greater than about 80%. The sensitivity may be greater than about 95% and the specificity may be greater than about 90%. The sensitivity may be greater than about 95% and the specificity may be greater than about 75%.

A method as described herein may (i) determine a presence of a condition, such as a lung cancer or (ii) classify a tissue as benign or malignant, such method may provide a negative predictive value (NPV) that may be greater than or equal to about 95%. The NPV may be at least about: 95%, 95.5%, 96%, 96.5%, 97%, 97.5%, 98%, 98.5%, 99%, 99.5% or more. In some cases, the NPV may be from about 95% to about 99%. In some cases, the NPV may be from about 96% to about 99%. In some cases, the NPV may be from about 97% to about 99%. In some cases, the NPV may be from about 98% to about 99%. In some cases, the NPV may be from about 95% to 100%. In some cases, the NPV may be from about 96% to 100%. In some cases, the NPV may be from about 97% to 100%. In some cases, the NPV may be from about 98% to 100%.

In some embodiments, the nominal specificity is greater than or equal to about 50%. In some embodiments, the nominal specificity is greater than or equal to about 60%. In some embodiments, the nominal specificity is greater than or equal to about 70%. In some embodiments, the nominal negative predictive value (NPV) is greater than or equal to about 95%. In some embodiments, the NPV is at least about: 90%, 91%, 92%, 93%, 94%, 95%, 95.5%, 96%, 96.5%, 97%, 97.5%, 98%, 98.5%, 99%, 99.5% (e.g., 90%, 91%, 92%, 93%, 94%, 95%, 95.5%, 96%, 96.5%, 97%, 97.5%, 98%, 98.5%, 99%, 99.5%, or 100%) and the specificity (or positive predictive value (PPV)) is at least about: 30%, 35%, 40%, 50%, 60%, 70%, 80%, 90%, 95%, 95.5%, 96%, 96.5%, 97%, 97.5%, 98%, 98.5%, 99%, or 99.5% (e.g., 30%, 35%, 40%, 50%, 60%, 70%, 80%, 90%, 95%, 95.5%, 96%, 96.5%, 97%, 97.5%, 98%, 98.5%, 99%, 99.5%, or 100%). In some cases the NPV is at least about 95%, and the specificity is at least about 50%. In some cases the NPV is at least about 95% and the specificity is at least about 70%. In some cases the NPV is at least about 95% and the specificity is at least about 75%. In some cases the NPV is at least about 95% and the specificity is at least about 80%.

Sensitivity may refer to TP/(TP+FN), where TP is true positive and FN is false negative. Number of Continued Indeterminate results divided by the total number of malignant results based on adjudicated histopathology diagnosis. Specificity typically refers to TN/(TN+FP), where TN is true negative and FP is false positive. The number of benign results divided by the total number of benign results based on adjudicated histopathology diagnosis. Positive Predictive Value (PPV): TP/(TP+FP); Negative Predictive Value (NPV): TN/(TN+FN).

The present methods and compositions also relate to the use of biomarker panels for purposes of identification, classification, diagnosis, or to otherwise characterize a biological sample. A panel may identify one or more of the following: a field of injury; a field of cancerization; a presence of a condition (such as ILD, COPD, or lung cancer); an increased risk of developing a condition; a presence of a disease recurrence; a reversal of a disease; a prevention of a disease; or any combination thereof. The methods and compositions may also use groups of biomarker panels. Often the pattern of levels of gene expression of biomarkers in a panel (also known as a signature such as an injury signature or a cancerization signature) may be determined and then may be used to evaluate the signature of the same panel of biomarkers in a biological sample, such as by a measure of similarity between the sample signature and the reference signature. In some embodiments, the method involves measuring (or obtaining) the levels of two or more gene expression products that may be within a biomarker panel and/or within a classification panel. For example, in some embodiments, a biomarker panel or a classification panel may contain at least about: 1, 2, 3, 4, 5, 6, 7, 8, 9, 10, 15, 20, 25, 30, 33, 35, 38, 40, 43, 45, 48, 50, 53, 58, 63, 65, 68, 100, 120, 140, 142, 145, 147, 150, 152, 157, 160, 162, 167, 175, 180, 185, 190, 195, 200, or 300 biomarkers. In some embodiments, a biomarker panel or a classification panel contains no greater than or equal to about: 1, 2, 3, 4, 5, 6, 7, 8, 9, 10, 15, 20, 25, 30, 33, 35, 38, 40, 43, 45, 48, 50, 53, 58, 63, 65, 68, 100, 120, 140, 142, 145, 147, 150, 152, 157, 160, 162, 167, 175, 180, 185, 190, 195, 200, or 300 biomarkers. In some embodiments, a biomarker panel or a classification panel contains from about 1 to about 500 biomarkers. In some embodiments, a biomarker panel or a classification panel contains from about 1 to about 400 biomarkers. In some embodiments, a biomarker panel or a classification panel contains from about 1 to about 300 biomarkers. In some embodiments, a biomarker panel or a classification panel contains from about 1 to about 200 biomarkers. In some embodiments, a biomarker panel or a classification panel contains from about 1 to about 100 biomarkers. In some embodiments, a biomarker panel or a classification panel contains from about 1 to about 500 biomarkers. In some embodiments, a biomarker panel or a classification panel contains from about 100 to about 500 biomarkers. In some embodiments, a biomarker panel or a classification panel contains from about 200 to about 500 biomarkers. In some embodiments, a biomarker panel or a classification panel contains from about 300 to about 500 biomarkers. In some embodiments, a biomarker panel or a classification panel contains from about 400 to about 500 biomarkers. In some embodiments, a classification panel contains at least about: 1, 2, 3, 4, 5, 6, 7, 8, 9, 10, 15, 20, or 25 different biomarker panels. In other embodiments, a classification panel contains no greater than or equal to about: 1, 2, 3, 4, 5, 6, 7, 8, 9, 10, 15, 20, or 25 different biomarker panels. A biomarker panel may comprise a panel of genes that may identify an injury signature, confirm a presence of an interstitial pneumonia pattern (UIP), identify a risk of developing a disease, identify a risk of disease recurrence, monitor a disease progression, or any combination thereof.

One or more risk factors that may increase a risk or likelihood of developing lung cancer may including smoking, exposure to environmental smoke (such as secondhand smoke), exposure to radon, exposure to industrial substances (such as asbestos, arsenic, diesel exhaust, mustard gas, uranium, beryllium, vinyl chloride, nickel chromates, coal products, chloromethyl ethers, gasoline), inherited or environmentally-acquired gene mutations, tuberculosis, exposure to air pollution, exposure to radiation (such as previous radiation therapy), a subject's age, having a secondary condition (such as chronic obstructive pulmonary disease (COPD)), interstitial lung disease (ILD), asthma, or others), consumption of a dietary supplement (such as beta carotene) or any combination thereof. A risk factor that may increase a risk or a likelihood of developing a lung cancer may comprise cigarette smoking, cigar smoking, pipe smoking, or any combination thereof.

A subject having one risk factor may identify the subject as an at-risk individual. A subject having two risk factors may identify the subject as an at-risk individual. A subject having three risk factors may identify the subject as an at-risk individual. Individual risk factors may not be weighted equally. The presence of a single risk factor, such as smoking, may identify the subject as an at-risk individual. The presence of a single risk factor, such as having a particular genetic mutation, may not be sufficient alone but needed in combination with other risk factors to identify the subject as an at-risk individual.

A subject may be given a questionnaire (written or computerized) to provide answers to one or more questions that assess the presence of one or more risk factors. A medical professional may request answers to one or more questions directly from a subject to assess the presence of one or more risk factors. A non-invasive sample may be provided by a subject to assess a presence of one or more risk factors. A previous medical history of a subject may be provided to assess a presence of one or more risk factors. A medical professional may retain health or physiological data of a subject, which may comprise, for example, a medical history of the subject.

An inconclusive diagnosis can lead to unnecessary surgery, delayed diagnosis, delayed treatment, or any combination thereof. In the current clinical pathway, from 15-70% of diagnosis may be uncertain or inconclusive. In the case of an inconclusive diagnosis, diagnostic surgery may be recommended. A portion of those subjects recommended for surgery, due to an inconclusive diagnosis, may be benign. Development of genomic classifiers that can diagnosis or classify a sample with high sensitivity and specificity may be needed.

Currently there may be about 225,000 new cases of lung cancer each year. In about 90% of these new cases, the subject may be identified as a smoker during at least a portion of their life. About 40% of subjects that undergo an invasive biopsy do not have cancer. Further, early detection may also be important to reducing mortality. However, current standards of care require invasive procedures to diagnose.

Lung tissue, such as peripheral lung nodules may be difficult to obtain a biopsy and can yield high rates of inconclusive or non-diagnostic bronchoscopies. Therefore, alternative options for diagnosing lung cancer may be desired.

Smoking may alter gene expression of epithelial cells throughout an airway including epithelial cells of the nose, mouth, oral cavity, nasal cavity, pharynx, larynx, trachea, lung, bronchus, alveolus, or any combination thereof.

Isolating epithelial cells from a portion of an airway and assaying for a gene signature or panel of biomarkers in the isolated epithelial cells may determine a risk of developing cancer or confirm a presence of cancer or classifying a lung tissue as benign or malignant. Such assaying may be performed, for example, using nucleic acid amplification (e.g., PCR), array hybridization or sequencing. Such sequencing may be massively parallel sequencing (e.g., Illumina, Pacific Biosciences of California, or Oxford Nanopore). Sequencing may provide sequencing reads, which may be used to identify genetic (or genomic) aberrations (e.g., copy number variation, single nucleotide polymorphism, single nucleotide variant, insertion or deletion, etc.) and an expression level corresponding to a gene or expression levels corresponding to genes. This may advantageously provide information relating to genetic aberrations in a genome of the subject together with information relating to a level of expression of a transcript messenger ribonucleic acid molecule (mRNA) from the same sample.

An isolated epithelial cell may be isolated from a section of an airway that may be distant from the site of a cancer or a tumor. For example, an isolated epithelial cell may be a nasal epithelial cell or an oral epithelial cell and a gene signature of expression level of a panel of biomarkers obtained from the isolated nasal epithelial cell may predict a risk of developing cancer or confirm a presence of cancer in a bronchial tissue or in a peripheral lung nodule. Tumor-specific genomic alternations may be present in the surrounding airway tissues. Genomic alterations associated with the presence of a cancer may be found in cells throughout an airway.

Subtypes of interstitial lung disease (ILD) may be difficult to differentiate and to diagnosis with clinical certainty. Many subjects having ILD, such as about 42%, report at least one year delay from initial symptoms to receiving a confirmed diagnosis. Misdiagnosis may be common. At least 55% of subjects having ILD report at least one misdiagnosis.

About 200,000 subjects in the US and Europe suspected of ILD may be evaluated each year. About 25-30% of subjects receiving a high-resolution CT scan show a presence of UIP. About 70-75% (about 150,000) subjects receive an uncertain or inconclusive diagnosis following high-resolution CT scan. These subjects receiving an inconclusive diagnosis may be recommended for diagnostic surgery.

There may be a need to develop a genomic classifier using gene signatures (such as class UIP pattern for IPF) to improve diagnostic accuracy and reduce the number of subjects receiving diagnostic surgery.

The methods described herein provide a genomic classifier to identify the presence of an ILD (such as IPF) by assaying for a biomarker panel (such as a classic UIP pattern) in a sample obtained from a subject suspected of having the ILD. The method may have at least about 88% specificity and at least about 67% sensitivity. For subjects having a positive UIP pattern identified by a genomic classifier, the percent of subjects having a subsequent diagnostic biopsy decreased from about 59% without use of the genomic classifier to about 29% with use of the genomic classifier.

High resolution computed tomography (HRCT) criteria for a classic UIP pattern may include at least four of: a subpleural basal predominance, a reticular abnormality, a honeycombing with or without traction bronchiectasis, and an absence of features listed as inconsistent with UIP pattern. A possible UIP pattern may include three of the following: subpleural basal predominance, a reticular abnormality, an absence of features listing as inconsistent with UIP pattern. Indications that may be inconsistent with a classic UIP pattern include any of the following: upper or mid-lung predominance, peribronchvascular predominance, extensive ground glass abnormality, profuse micronodules, discrete cysts, diffuse mosaic attenuation or air-trapping, consolidation of bronchopulmonary segments or lobes.

A subject (such as a subject at a low risk for developing a lung cancer) may receive a bronchoscopy, a transthoracic needle aspiration (TTNA), a video-assisted thoracic-scopic surgery (VATS) or other method to obtain an airway tissue sample, such as a lung tissue sample. If the bronchoscopy may be inconclusive or non-diagnostic, a classifier (such as a Bronchial Genomic Classifier) may be applied to identify and classify the airway tissue sample and avoid a further invasive procedure.

A subject may receive a biopsy, such as a transbronchial biopsy. A classifier (such as a Genomic Classifier) may be applied to one or more expression levels obtained from the biopsy to detect a presence or an absence of one or more genes of a panel of genes or a gene expression pattern (such as the classic IPF “UIP pattern”). A classifier may identify a presence or an absence of an ILD, such as IPF, in the biopsy.

For subjects who may be at an increased risk of developing lung cancer (based on one or more risk factors) as compared to the general population, a classifier (such as a Nasa-Detect classifier) may be employed to determine a presence or an absence of an “injury” signature in a subject that may be an early detection method for lung cancer diagnosis. A classifier (such as a Nasa-Detect classifier) may be applied to one or more expression levels assayed in a sample obtained from a subject to detect a presence or an absence of one or more genes of a panel of genes or a gene expression pattern. The panel of genes may comprise a signature of “injury” that may predispose a subject to develop a lung cancer or may be an early indicator of a presence of the disease. This classifier may be utilized to identify subjects that may be potential candidates for interventive therapy or injury reversal. If the classifier (such as the Nasa-Detect classifier) reports a negative result, that the subject does not have a presence or an altered expression of one or more genes of the “injury” panel, the classifier may be re-run on a second sample obtained from the subject at a later time point to monitor changes in gene expression. If the classifier (such as the Nasa-Detect classifier) reports a positive result, that the subject does have a presence or an altered expression of one or more genes of the “injury” panel, then a subject may receive a low-dose CT scan (LDCT).

A classifier may be trained to detect “injury” in “at-risk” populations of subjects. A positive result may include a recommendation for a follow-up investigation with a LDCT. A negative result may include a recommendation for monitoring with a second classifier (such as Nasa-Detect classifier) at a recurring time interval, such as about: every 0.5 year, every 1 year, every 1.5 years, every 2 years, every 2.5 years, every 3 years, every 3.5 years, every 4 years, every 4.5 years, or every 5 years, or longer. In some cases, a recurring time interval may be from about 0.5 year to about 3 years. In some cases, a recurring time interval may be from about 1 years to about 3 years. In some cases, a recurring time interval may be from about 2 years to about 3 years. In some cases, a recurring time interval may be from about 0.5 year to about 2 years. In some cases, a recurring time interval may be from about 0.5 year to about 1.5 years. A classifier trained to detect “injury” in “at-risk” populations may (i) optimize the subset of subjects that may be screened by an LDCT, (ii) augment LDCT screening with a specific screening tool, (iii) detect subjects that may benefit from interventive therapy, or any combination thereof.

A subject may receive a low-dose CT scan to determine a presence or absence of one or more lung nodules. If the LDCT shows an absence of lung nodules, (i) the classifier (such as the Nasa-Detect classifier) may be re-run on a second sample obtained from the subject at a later time point to monitor changes in gene expression of the one or more genes of the “injury” panel or (ii) the subject may be recommended for receiving an interventive therapy. If the LDCT shows a presence of one or more lung nodules, a classifier (such as a Nasa-Risk Stratifier classifier) may be applied to one or more expression levels assayed in a sample run obtained from a subject.

A subject recommended from interventive therapy (such as a subject with an absence of lung nodules as measured by LDCT), may receive one or more drug therapies. Following administering of one or more drug therapies, a sample may be obtained from the subject, assayed for one or more expression levels and run on a classifier (such as a Nasa-Protect Monitoring classifier). The classifier (such as the Nasa-Protect Monitoring classifier) may be trained to monitor changes of a particular set of biomarkers and to make a recommendation of whether to continue a particular drug regime. A result of the classifier (such as the Nasa-Protect Monitoring classifier) may be to recommend ceasing a drug therapy, switching to a different drug therapy, switching to a different non-drug therapy, maintaining a current therapy, or any combination thereof. A classifier (such as a Nasa-Protect Monitoring classifier) may be utilized as a companion diagnostic to monitor a reversal of a field of injury that may halt progression of a cancer, such as lung cancer.

A classifier (such as a Nasa-Protect classifier) may be trained as a companion diagnostic to monitor lung injury reversal. A classifier may be trained to identify a subset of subjects that may be benefiting from a particular treatment or drug regime.

When a LDCT yields a presence of one or more lung nodules, a sample may be obtained from a subject. The sample may be assayed for one or more expression levels and the one or more expression levels input into a classifier (such as a Nasa-Risk Stratifier classifier). A classifier (such as a Nasa-Risk Stratifier classifier) may be run prior to a bronchoscopy or other invasive procedure. A classifier (such as a Nasa-Risk Stratifier classifier) may identify a subject at low-risk for developing lung cancer, at high-risk for developing lung cancer, at low-risk of having lung cancer, or at high-risk of having lung cancer. When a result of the classifier (such as the Nasa-Risk Stratifier classifier) yields a low-risk result, another LDCT may be performed on the subject at a later point in time. When a result of the classifier (such as the Nasa-Risk Stratifier classifier) yields a high-risk result, then the subject may receive a bronchoscopy, a transthoracic needle aspiration (TTNA), a video-assisted thoracic-scopic surgery (VATS), or another invasive procedure. A classifier (such as a Nasa-Risk Stratifier classifier) may shift the course of next steps for a subject into two different categories (such as a subject with high-risk and a subject with low-risk). This shift in the course of next steps may improve early detection of cancer with a lower false positive.

A classifier (such as a Nasa-Risk Stratifier classifier) may be trained to stratify a risk of a presence of nodules, such as nodules detected by LDCT, to better inform next clinical steps. A classifier may include radiological selection features. A classifier may be developed on an Next-generation sequencing (NGS) platform. A classifier yielding a low-risk result, may include a recommendation of continued surveillance or monitoring of a subject or include a recommendation of a subject as a potential candidate for interventive therapy. A classifier yielding a high-risk result, may include a recommendation to proceed with a surgical biopsy. A classifier may accelerate surgical biopsy in those subjects that need further testing and avoid surgical biopsy in those subjects that do not. A classifier may minimize the number of indeterminate pulmonary nodules. A subject population for a classifier may include subjects having confirmed presence of pulmonary lesions, such as by LDCT.

In some cases, a bronchoscopy or other invasive procedure (such as TTNA or VATS) may yield a positive cancer diagnosis. In some cases, a bronchoscopy may yield a non-diagnostic result. In these cases, when a bronchoscopy may yield a non-diagnostic result, a sample may be obtained from the subject, assayed for one or more expression levels, and the expression levels may be input into a classifier (such as a Bronchial Genomic Classifier). If a classifier (such as a Bronchial Genomic Classifier) returns a result of intermediate risk, a subject may receive a second bronchoscopy or invasive procedure. If a classifier (such as a Bronchial Genomic Classifier) returns a result of low-risk, a subject may receive an interventive therapy or a second LDCT. In some cases, a bronchoscopy may yield a cancerous or malignant result. A subject receiving a cancerous or malignant result from a bronchoscopy or other invasive procedure may have the affected tissue surgically resected. If the affected tissue can be surgically resected, a sample may be obtained from a subject, assayed for one or more expression levels, and the expression levels may be input into a classifier (such as a Nasa-Recurrence classifier). After a cancer, such as an early stage cancer, may be detected and resected, a classifier (such as a Nasa-Recurrence classifier) may predict early recurrence through monitoring. If a result of a classifier (such as a Nasa-Recurrence classifier) may indicate no risk of recurrence than a second sample from the subject may be obtained at a later point in time, assayed for one or more expression levels, and the expression levels run through the classifier (such as the Nasa-Recurrence classifier). If a result of a classifier (such as a Nasa-Recurrence classifier) may indicate a risk of recurrence, a sample may be obtained from a subject and mutation testing, immune toxicology testing, or a combination thereof may be performed on the sample. Based on a result of the mutation or immunotx testing, a therapy may be recommended to a subject following by therapy monitoring and a second mutation or immunotx testing.

A classifier (such as a Nasa-Recurrence classifier) may be trained to non-invasively monitor subjects for a recurrence of cancer. A classifier may be trained to monitor subject that underwent curative surgical resection of a tumor for a recurrence of the tumor or cancer. In some cases, a classifier may indicate recurrence is detected or no recurrence is detected. A subject population may include subjects having received surgical resection to cure a lung cancer. A classifier may identify recurrence of disease in early stages.

If an affected tissue identified as cancerous or malignant cannot be surgically resected, a sample may be obtained from a subject and mutation or immunotx testing may be performing on the sample.

### Samples

One or more samples may be obtained from a subject. One or more samples may be a same type of sample, such as one or more biopsies. One or more samples obtained from a subject may be different types of samples, such as a biopsy and a fine needle aspiration.

A type of sample may include a blood sample, a tissue sample, or an image sample. A sample may comprise cell-free DNA. A blood sample may comprise cell-free DNA. A blood sample may comprise blood cells. A blood sample may comprise serum or plasma. A tissue sample may be obtained by surgical biopsy, surgical resection, needle aspiration, fine needle aspiration, a tissue swabbing, a tissue brushing or any combination thereof. A tissue sample may comprise epithelial cells, blood cells or a combination thereof. A tissue sample may comprise cancerous cells, non-cancerous cells, or a combination thereof. An image sample may be obtained by a bronchoscopy, a CT scan (such as a low-dose CT scan), a VATS, or a TTNA, or any combination thereof.

A sample may be an isolated and purified sample. A sample may be a freshly isolated sample. Cells from a freshly isolated sample may be isolated and cultures. A sample may comprise one or more cells. An isolated sample may comprise a heterogeneous mixture of cells. A sample may be purified to comprise a homogeneous mixture of cells. A sample may comprise about: 100 cells, 1,000 cells, 5,000 cells, 10,000 cells, 20,000 cells, 30,000 cells, 40,000 cells, 50,000 cells, 60,000 cells, 70,000 cells, 80,000 cells, 90,000 cells, 100,000 cells, 150,000 cells, 200,000 cells, 250,000 cells, 300,000 cells, 350,000 cells, 400,000 cells, 450,000 cells, 500,000 cells, 550,000 cells, 600,000 cells, 650,000 cells, 700,000 cells, 750,000 cells, 800,000 cells, 850,000 cells, 900,000 cells, 950,000 cells, or more. A sample may comprise from about 30,000 cells to about 1,000,000 cells. A sample may comprise from about 20,000 cells to about 50,000 cells. A sample may comprise from about 100,000 cells to about 400,000 cells. A sample may comprise from about 400,000 cells to about 800,000 cells.

A sample may comprise epithelial cells. A sample may comprise blood cells. A sample may comprise nasal tissue, oral tissue (gum tissue, cheek tissue, tongue tissue, or others), pharynx tissue, larynx tissue, trachea tissue, bronchi tissue, lung tissue, or any combination thereof.

A classifier may be trained with one or more training samples. A classifier may be trained with one or more different types of training samples. Different training sample types may comprise a surgical biopsy, a tissue resection, a needle aspiration, a fine needle aspiration, a blood sample, a cell-free DNA sample, an image or imaging data (such as a CT scan), or any combination thereof. A classifier may be trained with at least two different types of training samples, such as a surgical biopsy and a fine needle aspiration. A classifier may be trained with at least three different types of training samples, such as a surgical biopsy, fine needle aspiration, and blood sample. A classifier may be trained with at least three different types of training samples, such as a surgical biopsy, fine needle aspiration, and an image obtained from a CT scan. A classifier may be trained with at least four different types of training samples, such as a surgical biopsy, fine needle aspiration, a blood sample, and an image obtained from a CT scan.

Training samples may be obtained from one or more subjects. Subject may include subjects having a different country of birth. Subject may include subject having a different place of residence. Training samples may represent at least about: 2, 3, 4, 5, 6, 7, 8, 9, 10, 11, 12, 13, 14, 15, 16, 17, 18, 19, or 20 different countries of birth. Training samples may represent at least about 3 different countries of birth. Training samples may represent at least about 5 different countries of birth. Training samples may represent at least about 10 different countries of birth. Training samples may represent from about 2 to about 10 different countries of birth. Training samples may represent from about 3 to about 15 different countries of birth. Training samples may represent from about 2 to about 20 different countries of birth. Training samples may represent at least about: 2, 3, 4, 5, 6, 7, 8, 9, 10, 11, 12, 13, 14, 15, 16, 17, 18, 19, or 20 different countries of residence. Training samples may represent at least about 3 different countries of residence. Training samples may represent at least about 5 different countries of residence. Training samples may represent at least about 10 different countries of residence. Training samples may represent from about 2 to about 10 different countries of residence. Training samples may represent from about 3 to about 15 different countries of residence. Training samples may represent from about 2 to about 20 different countries of residence.

Training samples may comprise one or more samples obtained from a subject suspected of having a condition (such as lung cancer), a subject having a confirmed diagnosis of a condition (such as lung cancer), a subject having a pre-existing condition (such as a benign lung disease), a subject having lung nodules identified on a LDCT, a subject that may be a non-smoker, a subject that may be a non-smoker with environmental exposure to smoking, a current smoker, a previous smoker, a subject having smoked at least about: 1, 10, 20, 100, 200, 300, 400, 500, 600, 700, 800, 900, 1,000, 2,000, 3,000, 4,000, 5,000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000 or more cigarettes or cigars or e-cigarettes in their lifetime, a subject having an increased hereditary risk of developing a condition (such as lung cancer), a subject having a suppressed immune system, a subject having chronic pulmonary infections, or any combination thereof. In some cases, a subject may have smoked from about 1 to about 10 cigarettes, cigars, e-cigarettes in their lifetime. In some cases, a subject may have smoked from about 1 to about 100 cigarettes, cigars, e-cigarettes in their lifetime. In some cases, a subject may have smoked from about 1 to about 1000 cigarettes, cigars, e-cigarettes in their lifetime. In some cases, a subject may have smoked from about 1000 to about 10,000 cigarettes, cigars, e-cigarettes in their lifetime. In some cases, a subject may have smoked from about 10,000 to about 50,000 cigarettes, cigars, e-cigarettes in their lifetime. In some cases, a subject may have smoked from about 10,000 to about 100,000 cigarettes, cigars, e-cigarettes in their lifetime.

A smoker may be an individual having at least about: 1, 5, 10, 20, 30, 40, 50, 60, 70, 80, 90, 100, 200, 300, 400, or 500 cigarettes, cigars, or e-cigarettes in their lifetime. A smoker may be an individual having at least about 100 cigarettes, cigars, or e-cigarettes in their lifetime. A smoker may be an individual having at least about 500 cigarettes, cigars, or e-cigarettes in their lifetime. A smoker may be an individual having had greater than about: 5, 10, 20, 30, 40, or 50 packs of cigarettes, cigars, e-cigarettes per year. A smoker may be an individual having had greater than about 5 packs of cigarettes, cigars, e-cigarettes per year. A smoker may be an individual having had greater than about 10 packs of cigarettes, cigars, e-cigarettes per year. A smoker may be an individual having had greater than about 20 packs of cigarettes, cigars, e-cigarettes per year. A smoker may be an individual having had greater than about 30 packs of cigarettes, cigars, e-cigarettes per year. A smoker may be an individual having had from about 1 pack to about 12 packs (or more) of cigarettes, cigars, e-cigarettes per year. A smoker may be an individual having had from about 10 packs to about 25 packs of cigarettes, cigars, e-cigarettes per year. A smoker may be an individual having had from about 25 packs to about 50 packs of cigarettes, cigars, e-cigarettes per year. A smoker may be an individual having had from about 1 pack to about 50 packs of cigarettes, cigars, e-cigarettes per year. A smoker may be an individual having had from about 10 packs to about 50 packs of cigarettes, cigars, e-cigarettes per year.

Training samples may comprise one or more samples obtained from a smoker having received a positive diagnosis of a condition (such as lung cancer), a smoker having received a negative diagnosis of a condition (such as lung cancer), a smoker not having previously received a diagnosis, a non-smoker with environmental exposure having received a positive diagnosis of a condition (such as lung cancer), a non-smoker with environmental exposure having received a negative diagnosis of a condition (such as lung cancer), a non-smoker with environmental exposure not having previously received a diagnosis, a non-smoker having received a positive diagnosis of a condition (such as lung cancer), a non-smoker having received a negative diagnosis of a condition (such as lung cancer), a non-smoker not having previously received a diagnosis, or any combination thereof.

One or more types of genomic information may be obtained from a sample, such as a training sample or a validation sample. For example, a sample may be assayed for an expression level of one or more genes (such as genes of a biomarker panel). A sample may be assayed for a presence of an absence of one or more genes. A sample may be assayed for an expression level, a count or number of reads, a sequence variant, a fusion, a loss of heterozygosity (LOH), a mitochondrial transcript, one or more of any of these, or any combination thereof.

A sample may be collected from the same subject more than one time. For example, a first sample may be collected from a subject and a second sample may be collected about 1 year after the first sample has been collected. Samples may be collected from the same subject daily, multiple times a week, bi-weekly, weekly, bi-monthly, monthly, bi-yearly, yearly, every two years, every three years, every four years, or every five years. In some examples, a first sample is collected at a given point in time and at least a second sample is collected within a time period of 1 day, 2 days, 3 days, 4 days, 5 days, 6 days, 1 week, 2 weeks, 3 weeks, 1 month, 2 months, 3 months, 4 months, 5 months, 6 months, 1 year, 2 years, 3 years, 4 years, 5 years or more with respect to the given point in time. Results from the second sample may be compared to results of the first sample to monitor a disease progression in the subject, an efficacy of a prescribed treatment or therapy, or a change in a risk of developing a condition, or any combination thereof.

A classifier may be trained to spot one or more features. A feature may relate to a condition (such as a lung cancer), a tissue type (such as a lung tissue), a population (such as subjects of a similar genetic makeup), an exposure risk (such as an environmental pollution or exposure to cigarette or cigar smoke), an injury profile, or any combination thereof. A classifier may be part of a screening assay, a diagnostic assay, a treatment regime, a monitoring regime, or any combination thereof.

The present disclosure provides methods for storing a sample for a period of time, such as seconds, minutes, hours, days, weeks, months, years or longer, after the sample has been obtained and before the sample is analyzed by one or more methods of the present disclosure. In some cases, the sample obtained from a subject may be subdivided prior to the step of storage or further analysis such that different portions of the sample may be subject to different downstream methods or processes including but not limited to storage, cytological analysis, adequacy tests, nucleic acid extraction, molecular profiling or a combination thereof.

In some cases, a portion of the sample may be stored while another portion of the sample may be further manipulated. Such manipulations may include but may not be limited to molecular profiling; cytological staining; nucleic acid (RNA or DNA) extraction, detection, or quantification; gene expression product (RNA or Protein) extraction, detection, or quantification; fixation; and examination. The sample may be fixed prior to or during storage by any method known to the art such as using glutaraldehyde, formaldehyde, or methanol. In other cases, the sample is obtained and stored and subdivided after the step of storage for further analysis such that different portions of the sample may be subject to different downstream methods or processes including but not limited to storage, cytological analysis, adequacy tests, nucleic acid extraction, molecular profiling or a combination thereof. In some cases, samples may be obtained and analyzed by, for example cytological analysis, and the resulting sample material is further analyzed by one or more molecular profiling methods provided herein. In such cases, the samples may be stored between the steps of cytological analysis and the steps of molecular profiling. Samples may be stored upon acquisition to facilitate transport, or to wait for the results of other analyses. In another embodiment, samples may be stored while awaiting instructions from a physician or other medical professional.

Cytological assays mark the current diagnostic standard for many types of suspected tumors including for example thyroid tumors or nodules. In some embodiments of the present disclosure, samples that assay as negative, indeterminate, diagnostic, or non-diagnostic may be subjected to subsequent assays to obtain more information. In the present disclosure, these subsequent assays may comprise molecular profiling of genomic DNA, RNA, mRNA expression product levels, miRNA levels, gene expression product levels or gene expression product alternative splicing. In some embodiments of the present disclosure, molecular profiling refers to the determination of the number (e.g., copy number) and/or type of genomic DNA in a biological sample. In some cases, the number and/or type may further be compared to a control sample or a sample considered normal. In some embodiments, genomic DNA can be analyzed for copy number variation, such as an increase (amplification) or decrease in copy number, or variants, such as insertions, deletions, truncations and the like. Molecular profiling may be performed on the same sample, a portion of the same sample, or a new sample may be acquired using any of the methods described herein. The molecular profiling company may request additional sample by directly contacting the individual or through an intermediary such as a physician, third party testing center or laboratory, or a medical professional. In some cases, samples may be assayed using methods and compositions of the molecular profiling business in combination with some or all cytological staining or other diagnostic methods. In other cases, samples may be directly assayed using the methods and compositions of the molecular profiling business without the previous use of routine cytological staining or other diagnostic methods. In some cases the results of molecular profiling alone or in combination with cytology or other assays may enable those skilled in the art to diagnose or suggest treatment for the subject. In some cases, molecular profiling may be used alone or in combination with cytology to monitor tumors or suspected tumors over time for malignant changes.

The molecular profiling methods of the present disclosure provide for extracting and analyzing protein or nucleic acid (RNA or DNA) from one or more samples from a subject. In some cases, nucleic acid is extracted from the entire sample obtained. In other cases, nucleic acid is extracted from a portion of the sample obtained. In some cases, the portion of the sample not subjected to nucleic acid extraction may be analyzed by cytological examination or immuno-histochemistry. In some cases, multiple samples may be obtained from locations in close proximity to one another in a subject. For example, two different samples may be obtained from two different locations that are located at most about 500 millimeters (mm), 400 mm, 300 mm, 200 mm, 100 mm, 90 mm, 80 mm, 70 mm, 60 mm, 50 mm, 40 mm, 30 mm, 20 mm, 10 mm, 9 mm, 8 mm, 7 mm, 6 mm, 5 mm, 4 mm, 3 mm, 2 mm, 1 mm or less apart. In some cases multiple samples (e.g., obtained from proximate locations) may be analyzed by different methods. For example, a first sample may be analyzed by cytological examination or immuno-histochemistry, and a second sample may be analyzed via molecular profiling.

In some embodiments, the methods of the present disclosure comprise extracting nucleic acid molecules (e.g., DNA, RNA) from a tissue sample from a subject and generating a nucleic acid sequencing library. For example, a nucleic acid library may be generated by amplifying cDNA generated from isolated RNA by reverse transcription (RT-PCR). In some cases cDNA may be amplified by polymerase chain reaction (PCR).

### Classifiers

Intensity values for a sample can be analyzed using feature selection techniques including filter techniques which assess the relevance of features by looking at the intrinsic properties of the data, wrapper methods which embed the model hypothesis within a feature subset search, and embedded techniques in which the search for an optimal set of features may be built into a classifier algorithm.

Filter techniques useful in the methods of the present disclosure include (1) parametric methods such as the use of two sample t-tests, ANOVA analyses, Bayesian frameworks, and Gamma distribution models (2) model free methods such as the use of Wilcoxon rank sum tests, between-within class sum of squares tests, rank products methods, random permutation methods, or TNoM which involves setting a threshold point for fold-change differences in expression between two datasets and then detecting the threshold point in each gene that minimizes the number of misclassifications (3) and multivariate methods such as bivariate methods, correlation based feature selection methods (CFS), minimum redundancy maximum relevance methods (MRMR), Markov blanket filter methods, and uncorrelated shrunken centroid methods. Wrapper methods useful in the methods of the present disclosure include sequential search methods, genetic algorithms, and estimation of distribution algorithms. Embedded methods useful in the methods of the present disclosure include random forest algorithms, weight vector of support vector machine algorithms, and weights of logistic regression algorithms. Bioinformatics, 2007 Oct. 1; 23(19):2507-17 provides an overview of the relative merits of the filter techniques provided above for the analysis of intensity data.

Selected features may then be classified using a classifier algorithm. Illustrative algorithms include but may not be limited to methods that reduce the number of variables such as principal component analysis algorithms, partial least squares methods, and independent component analysis algorithms. Illustrative algorithms further include but may not be limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques include bagging procedures, boosting procedures, random forest algorithms, and combinations thereof. Cancer Inform, 2008; 6: 77-97 provides an overview of the classification techniques provided above for the analysis of microarray intensity data.

The subject methods and algorithms enable: 1) gene expression analysis of samples containing low amount and/or low quality of nucleic acid; 2) a significant reduction of false positives and false negatives, 3) a determination of the underlying genetic, metabolic, or signaling pathways responsible for the resulting pathology, 4) the ability to assign a statistical probability to the accuracy of a diagnosis, a risk of developing a condition, a monitoring of changes in a condition, an effectiveness of an interventive therapy, or combinations thereof, 5) the ability to resolve ambiguous results, and 6) the ability to distinguish between lung conditions or sub-types of lung conditions.

In some embodiments, the methods of the present disclosure provide for an upfront method of determining the cellular make-up of a particular biological sample so that the resulting molecular profiling signatures can be calibrated against the dilution effect due to the presence of other cell and/or tissue types. In one aspect, this upfront method may be an algorithm that uses a combination of known cell and/or tissue specific gene expression patterns as an upfront mini-classifier for each component of the sample. This algorithm utilizes this molecular fingerprint to pre-classify the samples according to their composition and then apply a correction/normalization factor. This data may in some cases then feed in to a final classification algorithm which may incorporate that information to aid in the final diagnosis.

Raw gene expression level and alternative splicing data may in some cases be improved through the application of algorithms designed to normalize and or improve the reliability of the data. In some embodiments of the present disclosure the data analysis requires a computer or other device, machine or apparatus for application of the various algorithms described herein due to the large number of individual data points that may be processed. A “machine learning algorithm” refers to a computational-based prediction methodology, also known to persons skilled in the art as a “classifier”, employed for characterizing a gene expression profile. The signals corresponding to certain expression levels, which may be obtained by, e.g., microarray-based hybridization assays, may be typically subjected to the algorithm in order to classify the expression profile. Supervised learning generally involves “training” a classifier to recognize the distinctions among classes and then “testing” the accuracy of the classifier on an independent test set. For new, unknown samples the classifier can be used to predict the class in which the samples belong.

In some cases, the robust multi-array Average (RMA) method may be used to normalize the raw data. The RMA method begins by computing background-corrected intensities for each matched cell on a number of microarrays. The background corrected values may be restricted to positive values as described by Irizarry et al. Biostatistics 2003 Apr. 4 (2): 249-64. After background correction, the base-2 logarithm of each background corrected matched-cell intensity may be then obtained. The back-ground corrected, log-transformed, matched intensity on each microarray may be then normalized using the quantile normalization method in which for each input array and each probe expression value, the array percentile probe value may be replaced with the average of all array percentile points, this method may be more completely described by Bolstad et al. Bioinformatics 2003. Following quantile normalization, the normalized data may then be fit to a linear model to obtain an expression measure for each probe on each microarray. Tukey's median polish algorithm (Tukey, J. W., Exploratory Data Analysis. 1977) may then be used to determine the log-scale expression level for the normalized probe set data.

Data may further be filtered to remove data that may be considered suspect. In some embodiments, data deriving from microarray probes that have fewer than about: 1, 2, 3, 4, 5, 6, 7 or 8 guanosine+cytosine nucleotides may be considered to be unreliable due to their aberrant hybridization propensity or secondary structure issues. A microarray probe having greater than or equal to about 4 guanosine+cytosine nucleotides may be considered unreliable. A microarray probe having greater than or equal to about 6 guanosine+cytosine nucleotides may be considered unreliable. A microarray probe having greater than or equal to about 8 guanosine+cytosine nucleotides may be considered unreliable. A microarray probe having from about 4 guanosine+cytosine nucleotides to about 8 guanosine+cytosine nucleotides may be considered unreliable. Similarly, data deriving from microarray probes that have greater than or equal to about: 8, 9, 10, 11, 12, 13, 14, 15, 16, 17, 18, 19, 20, 21, 22, 23, 24, 25 guanosine+cytosine nucleotides may be considered unreliable due to their aberrant hybridization propensity or secondary structure issues. A microarray probe having greater than or equal to about 10 guanosine+cytosine nucleotides may be unreliable. A microarray probe having greater than or equal to about 15 guanosine+cytosine nucleotides may be unreliable. A microarray probe having greater than or equal to about 20 guanosine+cytosine nucleotides may be unreliable. A microarray probe having greater than or equal to about 25 guanosine+cytosine nucleotides may be unreliable. A microarray probe having from about 8 guanosine+cytosine nucleotides to about 30 guanosine+cytosine nucleotides may be unreliable. A microarray probe having from about 10 guanosine+cytosine nucleotides to about 30 guanosine+cytosine nucleotides may be unreliable. A microarray probe having from about 12 guanosine+cytosine nucleotides to about 30 guanosine+cytosine nucleotides may be unreliable. A microarray probe having from about 15 guanosine+cytosine nucleotides to about 30 guanosine+cytosine nucleotides may be unreliable.

In some cases, unreliable probe sets may be selected for exclusion from data analysis by ranking probe-set reliability against a series of reference datasets. For example, RefSeq or Ensembl (EMBL) may be considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion. In some cases, the Ensembl high throughput cDNA and/or mRNA reference datasets may be used to determine the probe-set reliability separately or together. In other cases, probe-set reliability may be ranked. For example, probes and/or probe-sets that match perfectly to all reference datasets may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there may be many methods understood in the art for assessing the reliability of a given probe and/or probe-set for molecular profiling and the methods of the present disclosure encompass any of these methods and combinations thereof.

Methods of data analysis of gene expression levels or of alternative splicing may further include the use of a feature selection algorithm as provided herein. In some embodiments of the present disclosure, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420).

Methods of data analysis of gene expression levels and or of alternative splicing may further include the use of a pre-classifier algorithm. For example, an algorithm may use a cell-specific molecular fingerprint to pre-classify the samples according to their composition and then apply a correction/normalization factor. This data/information may then be fed in to a final classification algorithm which may incorporate that information to aid in the final diagnosis or prognosis, or monitoring evaluation.

Methods of data analysis of gene expression levels and or of alternative splicing may further include the use of a classifier algorithm as provided herein. In some embodiments of the present disclosure a support vector machine (SVM) algorithm, a random forest algorithm, or a combination thereof is provided for classification of microarray data. In some embodiments, identified markers that distinguish samples (e.g., benign vs. malignant, normal vs. malignant, low risk vs. high risk) or distinguish types (e.g., ILD vs. lung cancer) may selected based on statistical significance. In some cases, the statistical significance selection is performed after applying a Benjamini Hochberg correction for false discovery rate (FDR).

In some cases, the classifier algorithm may be supplemented with a meta-analysis approach such as that described by Fishel and Kaufman et al. 2007 Bioinformatics 23(13): 1599-606. In some cases, the classifier algorithm may be supplemented with a meta-analysis approach such as a repeatability analysis. In some cases, the repeatability analysis selects markers that appear in at least one predictive expression product marker set.

In some cases, the results of feature selection and classification may be ranked using a Bayesian post-analysis method. For example, microarray data may be extracted, normalized, and summarized using methods known in the art such as the methods provided herein. The data may then be subjected to a feature selection step such as any feature selection methods known in the art such as the methods provided herein including but not limited to the feature selection methods provided in LIMMA. The data may then be subjected to a classification step such as any of the classification methods known in the art such as the use of any of the algorithms or methods provided herein including but not limited to the use of SVM or random forest algorithms. The results of the classifier algorithm may then be ranked by according to a posterior probability function. For example, the posterior probability function may be derived from examining known molecular profiling results, such as published results, to derive prior probabilities from type I and type II error rates of assigning a marker to a category (e.g., ILD, COPD, lung cancer etc.). These error rates may be calculated based on reported sample size for each study using an estimated fold change value (e.g., 1.1, 1.2, 1.3, 1.4, 1.5, 1.6, 1.7, 1.8, 1.9, 2, 2.2, 2.4, 2.5, 3, 4, 5, 6, 7, 8, 9, 10 or more). A fold change value may be about: 0.5, 0.8, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, or 10.0. A fold change value may be from about 0.5 to about 10.0. A fold change value may be from about 0.5 to about 1.0. A fold change value may be from about 0.5 to about 5.0. A fold change value may be from about 2.0 to about 8.0. A fold change value may be from about 2.0 to about 6.0. A fold change value may be from about 6.0 to about 10.0. A fold change value may be from about 5.0 to about 10.0. A fold change value may be from about 8.0 to about 10.0. These prior probabilities may then be combined with a molecular profiling dataset of the present disclosure to estimate the posterior probability of differential gene expression. Finally, the posterior probability estimates may be combined with a second dataset of the present disclosure to formulate the final posterior probabilities of differential expression. Additional methods for deriving and applying posterior probabilities to the analysis of microarray data may be known in the art and have been described for example in Smyth, G. K. 2004 Stat. Appl. Genet. Mol. Biol. 3: Article 3. In some cases, the posterior probabilities may be used to rank the markers provided by the classifier algorithm. In some cases, markers may be ranked according to their posterior probabilities and those that pass a chosen threshold may be chosen as markers whose differential expression is indicative of or diagnostic for samples that may be for example benign, malignant, normal, low risk, high risk, or condition type (ILD, COPD, lung cancer). Illustrative threshold values include prior probabilities of at least about: 0.7, 0.75, 0.8, 0.85, 0.9, 0.925, 0.95, 0.975, 0.98, 0.985, 0.99, 0.995 or higher. A probability may be at least about 0.7. A probability may be at least about 0.75. A probability may be at least about 0.8. A probability may be at least about 0.85. A probability may be at least about 0.9. A probability may be at least about 0.95. A probability may be at least about 0.99. A probability may be from about 0.75 to about 0.995. A probability may be from about 0.80 to about 0.995. A probability may be from about 0.85 to about 0.995. A probability may be from about 0.9 to about 0.995. A probability may be from about 0.85 to about 0.95. A probability may be from about 0.8 to about 0.95. A probability may be from about 0.75 to about 0.95.

A statistical evaluation of the results of the molecular profiling may provide a quantitative value or values indicative of one or more of the following: the likelihood of diagnostic accuracy, the likelihood of cancer, disease or condition, the likelihood of a particular cancer, disease or condition, the likelihood of the success of a particular therapeutic intervention. Thus a physician, who may not be likely to be trained in genetics or molecular biology, need not understand the raw data. Rather, the data may be presented directly to the physician in its most useful form to guide patient care. The results of the molecular profiling can be statistically evaluated using a number of methods known to the art including, but not limited to: the students T test, the two sided T test, pearson rank sum analysis, hidden markov model analysis, analysis of q-q plots, principal component analysis, one way ANOVA, two way ANOVA, LIMMA and the like.

In some embodiments of the present disclosure, results may be classified using a trained algorithm. Trained algorithms of the present disclosure include algorithms that have been developed using a reference set of known malignant, benign, and normal samples. Training samples may comprise FNA samples, surgical biopsy samples, bronchoscope samples, or any combination thereof. Algorithms suitable for categorization of samples include but may not be limited to k-nearest neighbor algorithms, concept vector algorithms, naive bayesian algorithms, neural network algorithms, hidden markov model algorithms, genetic algorithms, and mutual information feature selection algorithms or any combination thereof. In some cases, trained algorithms of the present disclosure may incorporate data other than gene expression or alternative splicing data such as but not limited to DNA polymorphism data, sequencing data, scoring or diagnosis by cytologists or pathologists of the present disclosure, information provided by the pre-classifier algorithm of the present disclosure, or information about the medical history of the subject of the present disclosure.

Classifiers used early in the sequential analysis may be used to either rule-in or rule-out a sample as benign or suspicious or a sample as low-risk or high-risk or samples having ILD from samples not having ILD. In some embodiments, such sequential analysis ends with the application of a “main” classifier to data from samples that have not been ruled out by the preceding classifiers, wherein the main classifier may be obtained from data analysis of gene expression levels in multiple types of tissue and wherein the main classifier may be capable of designating the sample as benign or suspicious (or malignant).

In the next step of the example classification process, a first comparison may be made between the gene expression level(s) of the sample and the first set of biomarkers or first classifier. If the result of this first comparison is a match, the classification process ends with a result, such as designating the sample as low risk or high risk for developing a lung condition or for identifying samples having ILD vs. lung cancer. If the result of the comparison is not a match, the gene expression level(s) of the sample may be compared in a second round of comparison to a second set of biomarkers or second classifier. If the result of this second comparison is a match, the classification process ends with a result, such as (a) reporting a diagnosis to a subject with a lung condition, (b) reporting a risk of developing a lung condition, (c) reporting an effectiveness of an interventive therapy, (d) recommending a follow-on procedure such as an imaging scan, another sample acquisition, a bronchoscopy, a biopsy, a surgical resection, a pharmaceutical composition. If the result of the comparison is not a match, the process continues in a similar stepwise process of comparisons until a match is found, or until all sets of biomarkers or classifiers included in the classification process may be used as a basis of comparison. In some embodiments, the final comparison in the classification process is between the gene expression level(s) of the sample and a main classifier, as described herein.

In some cases, a method may employ more than one machine learning algorithm. For example, a method may employ about: 2, 3, 4, 5, 6, 7, 8, 9, 10, 11, 12, 13, 14, 15, 16, 17, 18, 19, 20 machine learning algorithms or more. In some cases, a method may employ at least about 4 machine learning algorithms. In some cases, a method may employ at least about 5 machine learning algorithms. In some cases, a method may employ at least about 6 machine learning algorithms. In some cases, a method may employ at least about 7 machine learning algorithms. In some cases, a method may employ at least about 8 machine learning algorithms. In some cases, a method may employ at least about 9 machine learning algorithms. In some cases, a method may employ at least about 10 machine learning algorithms. In some cases, a method may employ from about 4 machine learning algorithms to about 10 machine learning algorithms. In some cases, a method may employ from about 6 machine learning algorithms to about 10 machine learning algorithms. In some cases, a method may employ from about 4 machine learning algorithms to about 8 machine learning algorithms. In some cases, a method may employ from about 4 machine learning algorithms to about 15 machine learning algorithms. A method may employ more than one machine learning algorithm in a sequential manner. In some cases, a method may employ a mixture of machine learning algorithms and fusion calling algorithms. For example, a method may employ at least one machine learning algorithm and at least one fusion calling algorithm. In some cases, a method may employ at least 5 machine learning algorithms and at least one fusion calling algorithm. In some cases, a method may employ at least 7 machine learning algorithms and at least one fusion calling algorithm.

The present methods and systems may identify a presence or an absence of one or more biomarkers in a sample. For example, biomarkers may comprise biomarkers from Tables 1, 2, 3, 4, 5, 6, 7, 8, 9, 10, 11, 12, 13, or any combination thereof. In some cases, biomarkers may comprise biomarkers from Table 1, Table 2, or a combination thereof. In some cases, biomarkers may comprise biomarkers from Table 1, Table 2, Table 3, or any combination thereof. In some cases, biomarkers may comprise biomarkers from Table 4, Table 5, Table 6, Table 7, or any combination thereof. In some cases, biomarkers may comprise biomarkers from Table 8, Table 9, Table 10, or any combination thereof. In some cases, biomarkers may comprise biomarkers from Table 11, Table 12, Table 13, or any combination thereof. In some cases, biomarkers may comprise biomarkers from Table 1 or any combination thereof. In some cases, biomarkers may comprise biomarkers from Table 2 or any combination thereof. In some cases, biomarkers may comprise biomarkers from Table 3 or any combination thereof. In some cases, biomarkers may comprise biomarkers from Table 4 or any combination thereof. In some cases, biomarkers may comprise biomarkers from Table 5 or any combination thereof. In some cases, biomarkers may comprise biomarkers from Table 6 or any combination thereof. In some cases, biomarkers may comprise biomarkers from Table 7 or any combination thereof. In some cases, biomarkers may comprise biomarkers from Table 8 or any combination thereof. In some cases, biomarkers may comprise biomarkers from Table 9 or any combination thereof. In some cases, biomarkers may comprise biomarkers from Table 10 or any combination thereof. In some cases, biomarkers may comprise biomarkers from Table 11 or any combination thereof. In some cases, biomarkers may comprise biomarkers from Table 12 or any combination thereof. In some cases, biomarkers may comprise biomarkers from Table 13 or any combination thereof.

A presence or an absence or a differential expression of one or more biomarkers may be indicative of a presence of one or more risk factors for developing a condition, such as a lung cancer, IPF, ILD, COPD, or any combination thereof. A presence or an absence or a differential expression of one or more biomarkers may identify an effectiveness of an inventive therapy for preventing or reversing a condition (such as a lung cancer, IPF, ILD, COPD). A presence or an absence or a differential expression of one or more biomarkers may identify a risk or a presence of remission of a condition (such as a lung cancer, IPF, ILD, COPD) in a subject. A presence or an absence or a differential expression of one or more biomarkers may distinguish a smoker with condition from a smoker without a condition (such as lung cancer, IPF, ILD, COPD). A presence or an absence or a differential expression of one or more biomarkers may identify a diagnosis of a condition (such as lung cancer, IPF, ILD, COPD), a prognosis of a condition (such as lung cancer, IPF, ILD, COPD), or a combination thereof. A presence or an absence or a differential expression of one or more biomarkers may identify a field of injury. A presence or an absence or a differential expression of one or more biomarkers may identify a relationship between expression profiles of a first cell type or a first cell obtained from a first location and a second cell type or a second cell obtained from a second location. For example, a presence or an absence or a differential expression of one or more biomarkers in a nasal tissue may be indicative of a presence of a condition (such as lung cancer, IPF, ILD, COPD) in a bronchial tissue.

TABLE 1
Examples of biomarkers that may be up-regulated in IPF

| | |
|---|---|
| Mmp7/MMP7 | Matrix metallopeptidase 7 (matrilysin, uterine) |
| Pla2g2a/PLA2G2A | Phospholipase A2, group IIA (platelets, synovial fluid) |
| Lcn2/LCN2 | Lipocalin 2 |
| Cthrc1/CTHRC1 | Collagen triple helix repeat containing 1 |
| C6/C6 | Complement component 6 |
| Ctse/CTSE | Cathepsin E |
| Dclk1 /DCLK1 | Double cortin-like kinase 1 |
| Anln/ANLN | Anillin, actin binding protein |
| Kcnn4/KCNN4 | Potassium intermediate/small conductance calcium-activated channel, subfam- |
| | ily N, member 4 |
| Aspn/ASPN | Asporin |
| Pkib/PKIB | Protein kinase (cAMP-dependent, catalytic) inhibitor β |
| Fhl2/FHL2 | Four and a half LIM domains 2 |
| Mnd1/MND1 | Meiotic nuclear divisions 1 homolog (Saccharomyces cerevisiae) |
| Mycn/MYCN | V-myc myelocytomatosis viral related oncogene, neuroblastoma derived |
| | (avian) |
| Calca/CALCA | Calcitonin-related polypeptide α |
| Slc2a5/SLC2A5 | Solute carrier family 2 (facilitated glucose/fructose transporter), member 5 |
| Fkbp11/FKBP11 | FK506 binding protein 11, 19 kD |
| Gdf15/GDF15 | Growth differentiation factor 15 |
| Gal/GAL | Galanin prepropeptide |
| Top2a/TOP2A | Topoisomerase (DNA) II α, 170 kD |
| Tmem213/TMEM213 | Transmembrane protein 213 |
| Podnl1/PODNL1 | Podocan-like 1 |
| Pln/PLN | Phospholamban |
| Mia/MIA | Melanoma inhibitory activity |
| Bik/BIK | BCL2-interacting killer (apoptosis inducing) |
| Col1a2/COL1A2 | Collagen, type I, α 2 |
| Ccnb2/CCNB2 | Cyclin B2 |
| MGC105649/C15orf48 | Chromosome 15 open reading frame 48 |
| Ptges/PTGES | Prostaglandin E synthase |
| Ctsk/CTSK | Cathepsin K |
| Nuf2/NUF2 | NUF2, NDC80 kinetochore complex component, homolog (S. cerevisiae) |
| Bub1b/BUB1B | Budding uninhibited by benzimidazoles 1 homolog β (yeast) |
| Fap/FAP | Fibroblast activation protein, α |
| Col5a1/COL5A1 | Collagen, type V, α 1 |
| Fkbp10/FKBP10 | FK506 binding protein 10, 65 kD |
| Uchl1/UCHL1 | Ubiquitin carboxyl-terminal esterase Ll (ubiquitin thiolesterase) |
| Pla2g7/PLA2G7 | Phospholipase A2, group VII (platelet-activating factor acetylhydrolase, |
| | plasma) |
| Spc25/SPC25 | SPC25, NDC80 kinetochore complex component, homolog (S. cerevisiae) |
| Mlf1ip/MLFlIP | MLF1 interacting protein |
| Sel1l3/SEL1L3 | sel-1 suppressor of lin-12-like 3 (Caenorhabditis elegans) |
| Foxm1/FOXM1 | Forkhead box M1 |

TABLE 2
Examples of biomarkers that may be down regulated in IPF

| | |
|---|---|
| Esm1/ESM1 | Endothelial cell-specific molecule 1 |
| Tmem100/TMEM100 | Transmembrane protein 100 |
| Stxbp6/STXBP6 | Syntaxin binding protein 6 (amisyn) |
| Gcom1/GCOM1 | GRINL1A complex locus 1 |
| Hpgd/HPGD | Hydroxyprostaglandin dehydrogenase 15-(NAD) |
| Vegfa/VEGFA | Vascular endothelial growth factor A |
| Mme/MME | Membrane metallo-endopeptidase |
| Emp2/EMP2 | Epithelial membrane protein 2 |
| Slc1a1/SLC1A1 | Solute carrier family 1 (neuronal/epithelial high affinity glutamate transporter, |
| | system Xag), member 1 |
| Clic5/CLIC5 | Chloride intracellular channel 5 |
| Ptprr/PTPRR | Protein tyrosine phosphatase, receptor type, R |
| Anxa3/ANXA3 | Annexin A3 |
| Lrrn3/LRRN3 | Leucine rich repeat neuronal 3 |
| Rapgef5/RAPGEF5 | Rap guanine nucleotide exchange factor (GEF) 5 |
| Olfml2a/OLFML2A | Olfactomedin-like 2A |
| Sgef/ARHGEF26 | Rho guanine nucleotide exchange factor (GEF) 26 |
| Sdpr/SDPR | Serum deprivation response |
| Adrb2/ADRB2 | Adrenoceptor β 2, surface |
| Ramp2/RAMP2 | Receptor (G protein-coupled) activity modifying protein 2 |
| Ccdc68/CCDC68 | Coiled-coil domain containing 68 |
| RGD1306437/C13orf1 | NA |
| Cav2/CAV2 | Caveolin 2 |
| Npr3/NPR3 | Natriuretic peptide receptor C/guanylate cyclase C |
| Tal1/TAL1 | T cell acute lymphocytic leukemia 1 |
| Lifr/LIFR | Leukemia inhibitory factor receptor α |
| Prkce/PRKCE | Protein kinase C, ε |
| Cav1/CAV1 | Caveolin 1, caveolae protein, 22 kD |
| RGD1311307/C6orf145 | NA |
| Nebl/NEBL | Nebulette |
| Nedd9/NEDD9 | Neural precursor cell expressed, developmentally down-regulated 9 |
| S1pr5/S1PR5 | Sphingosine- 1-phosphate receptor 5 |
| Afap1l1/AFAP1L1 | Actin filament associated protein 1-like 1 |
| Thbd/THBD | Thrombomodulin |
| Pard6b/PARD6B | Par-6 partitioning defective 6 homolog β (C. elegans) |
| Radil/RADIL | Ras association and DIL domains |
| Dnase2b/DNASE2B | DeoxyRNase II β |
| LOC691221/C5orf4 | Chromosome 5 open reading frame 4 |
| Sh3bp5/SH3BP5 | SH3-domain binding protein 5 (BTK-associated) |
| Fgg/FGG | Fibrinogen γ chain |
| Epb4.115/EPB41L5 | Erythrocyte membrane protein band 4.1-like 5 |
| Tspan12/TSPAN12 | Tetraspanin 12 |
| Slc4a1/SLC4A1 | Solute carrier family 4, anion exchanger, member 1 |
| | (erythrocyte membrane protein band 3, Diego blood group) |
| Zfp365/ZNF365 | Zinc finger protein 365 |
| Phactrl/PHACTR1 | Phosphatase and actin regulator 1 |
| Gpdl/GPD1 | Glycerol-3-phosphate dehydrogenase 1 (soluble) |
| Veph1NEPH1 | Ventricular zone expressed PH domain homolog 1 (zebrafish) |
| Selenbp1/SELENBP1 | Selenium binding protein 1 |

TABLE 3
Examples of biomarkers that may be
differentially expressed in COPD
Gene
PCDH7
CCDC81
CEACAM5
PTPRH
C12orf36
B3GNT6
PLAG1
PDE7B
CACHD1
EPB41L2
FRNID4A
PRKCE
SULF1
TLE1
FAM114A1
ELF5
SGCE
SEC14L3
GPR155
ITGA9
PTGFR
ISLR
SLC5A7
ZNF483
DPYSL3
TNS3
FMNL2
GALE
CNTN3
HSD17B13
PTPRM
HLF
PROS1
PLA2G4A
KAL1
TCN1
DPP4
GPR98
KCNA1
CABLES1
PEG10
PPP1R9A
POLA2
C17orf37
ABCC4
CA8
CYP2A13
SETBP1
ANKS1B
CHP
THSD4
MPDU1
CD109
STK32A
HLHLA2
AMMECR1
NPAS3
GXYLT2
KLF12
CA12
C21orf121
SH3BP4
FABP6
GUCY1B3
FUT3
STX10
FTO
CNIN4
ATP8A1
GMDS
ZNF671
WBP5
MYO5B
FLRT3
SCGB1A1
SCNN1G
CFTR
LOC339524
THSD7A
CACNB4
DQX1
GLI3
NFAT5
RUNX1T1
SNTB1
C16orf89
PRKD1
ANXA6
YIPF1
ATP10B
HK2
ABHD2
DNAH5
GGT7
FBN1
PRSS12
TMPRSS4
AMIGO2
TMEM54
CAPRIN2

TABLE 4
Examples of biomarkers that may distinguish smokers
with lung cancer from smokers without lung cancer.

| Affymetrix ID | GenBank ID | Gene Name |
|---|---|---|
| 1316_at | NM_003335 | UBE1L |
| 200654_at | NM_000918 | P4HB |
| 200877_at | NM_006430.1 | CCT4 |
| 201530_x_at | NM_001416.1 | EIF4A1 |
| 201537_s_at | NM_004090 | DUSP3 |
| 201923_at | NM_006406.1 | PRDX4 |
| 202004_x_at | NM_003001.2 | SDHC |
| 202573_at | NM_001319 | CSNK1G2 |
| 203246_s_at | NM_006545.1 | TUSC4 |
| 203301_s_at | NM_021145.1 | DMTF1 |
| 203466_at | NM_002437.1 | MPV17 |
| 203588_s_at | NM_006286 | TFDP2 |
| 203704_s_at | NM_001003698 /// | RREB1 |
| | NM_001003699 /// | |
| | NM_002955 | |
| 204119_s_at | NM_001123 /// | ADK |
| | NM_006721 | |
| 204216_s_at | NM_024824 | FLJ11806 |
| 204247_s_at | NM_004935.1 | CDK5 |
| 204461_x_at | NM_002853.1 | RAD1 |
| 205010_at | NM_019067.1 | FLJ10613 |
| 205238_at | NM_024917.1 | CXorf34 |
| 205367_at | NM_020979.1 | APS |
| 206929_s_at | NM_005597.1 | NFIC |
| 207020_at | NM_007031.1 | HSF2BP |
| 207064_s_at | NM_009590.1 | AOC2 |
| 207283_at | NM_020217.1 | DKFZp547I014 |
| 207287_at | NM_025026.1 | FLJ14107 |
| 207365_x_at | NM_014709.1 | USP34 |
| 207436_x_at | NM_014896.1 | KIAA0894 |
| 207953_at | AF010144 | — |
| 207984_s_at | NM_005374.1 | MPP2 |
| 208678_at | NM_001696 | ATP6V1E1 |
| 209015_s_at | NM_005494 /// | DNAJB6 |
| | NM_058246 | |
| 209061_at | NM_006534 /// | NCOA3 |
| | NM_181659 | |
| 209432_s_at | NM_006368 | CREB3 |
| 209653_at | NM_002268 /// | KPNA4 |
| | NM_032771 | |
| 209703_x_at | NM_014033 | DKFZP586A0522 |
| 209746_s_at | NM_016138 | COQ7 |
| 209770_at | NM_007048 /// | BTN3A1 |
| | NM_194441 | |
| 210434_x_at | NM_006694 | JTB |
| 210858_x_at | NM_000051 /// | ATM |
| | NM_138292 /// | |
| | NM_138293 | |
| 211328_x_at | NM_000410 /// | HFE |
| | NM_139002 /// | |
| | NM_139003 /// | |
| | NM_139004 /// | |
| | NM_139005 /// | |
| | NM_139006 /// | |
| | NM_139007 /// | |
| | NM_139008 /// | |
| | NM_139009 /// | |
| | NM_139010 /// | |
| | NM_139011 | |
| 212041_at | NM_004691 | ATP6V0D1 |
| 212517_at | NM_012070 /// | ATRN |
| | NM_139321 /// | |
| | NM_139322 | |
| 213106_at | NM_006095 | ATP8A1 |
| 213212_x_at | AI632181 | — |
| 213919_at | AW024467 | — |
| 214153_at | NM_021814 | ELOVL5 |
| 214599_at | NM_005547.1 | IVL |
| 214722_at | NM_203458 | N2N |
| 214763_at | NM_015547 /// | THEA |
| | NM_147161 | |
| 214833_at | AB007958.1 | KIAA0792 |
| 214902_x_at | NM_207488 | FLJ42393 |
| 215067_x_at | NM_005809 /// | PRDX2 |
| | NM_181737 /// | |
| | NM_181738 | |
| 215336_at | NM_016248 /// | AKAP11 |
| | NM_144490 | |
| 215373_x_at | AK022213.1 | FLJ12151 |
| 215387_x_at | NM_005708 | GPC6 |
| 215600_x_at | NM_207102 | FBXW12 |
| 215609_at | AK023895 | — |
| 215645_at | NM_144606 /// | FLCN |
| | NM_144997 | |
| 215659_at | NM_018530 | GSDML |
| 215892_at | AK021474 | — |
| 216012_at | U43604.1 | — |
| 216110_x_at | AU147017 | — |
| 216187_x_at | AF222691.1 | LNX1 |
| 216745_x_at | NM_015116 | LRCH1 |
| 216922_x_at | NM_001005375 /// | DAZ2 |
| | NM_001005785 /// | |
| | NM_001005786 /// | |
| | NM_004081 /// | |
| | NM_020363 /// | |
| | NM_020364 /// | |
| | NM_020420 | |
| 217313_at | AC004692 | — |
| 217336_at | NM_001014 | RPS10 |
| 217371_s_at | NM_000585 /// | IL15 |
| | NM_172174 /// | |
| | NM_172175 | |
| 217588_at | NM_054020 /// | CATSPER2 |
| | NM_172095 /// | |
| | NM_172096 /// | |
| | NM_172097 | |
| 217671_at | BE466926 | — |
| 218067_s_at | NM_018011 | FLJ10154 |
| 218265_at | NM_024077 | SECISBP2 |
| 218336_at | NM_012394 | PFDN2 |
| 218425_at | NM_019011 /// | TRIAD3 |
| | NM_207111 /// | |
| | NM_207116 | |
| 218617_at | NM_017646 | TRIT1 |
| 218976_at | NM_021800 | DNAJC12 |
| 219203_at | NM_016049 | C14orf122 |
| 219290_x_at | NM_014395 | DAPP1 |
| 219977_at | NM_014336 | AIPL1 |
| 220071_x_at | NM_018097 | C15orf25 |
| 220113_x_at | NM_019014 | POLR1B |
| 220215_at | NM_024804 | FLJ12606 |
| 220242_x_at | NM_018260 | FLJ10891 |
| 220459_at | NM_018118 | MCM3APAS |
| 220856_x_at | NM_014128 | |
| 220934_s_at | NM_024084 | MGC3196 |
| 221294_at | NM_005294 | GPR21 |
| 221616_s_at | AF077053 | PGK1 |
| 221759_at | NM_138387 | G6PC3 |
| 222155_s_at | NM_024531 | GPR172A |
| 222168_at | NM_000693 | ALDH1A3 |
| 22223l_s_at | NM_018509 | PRO1855 |
| 222272_x_at | NM_033128 | SCIN |
| 222310_at | NM_020706 | SFRS15 |
| 222358_x_at | AI523613 | |
| 64371_at | NM_014884 | SFRS14 |

TABLE 5
Examples of biomarkers that may distinguish
smokers with cancer from smokers without cancer.

| GenBank ID | Gene Name | Affymetrix ID |
|---|---|---|
| NM_030757.1 | MKRN4 | 208082_x_at |
| R83000 | BTF3 | 214800_x_at |
| AK021571.1 | MUC20 | 215208_x_at |
| NM_014182.1 | ORMDL2 | 218556_at |
| NM_17932.1 | FLJ20700 | 207730_x_at |
| U85430.1 | NFATC3 | 210556_at |
| AI683552 | — | 217679_x_at |
| BC002642.1 | CTSS | 202901_x_at |
| AW024467 | RIPX | 213939_s_at |
| NM_030972.1 | MGC5384 | 208137_x_at |
| BC021135.1 | INADL | 214705_at |
| AL161952.1 | GLUL | 215001_s_at |
| AK026565.1 | FLJ10534 | 218155_x_at |
| AK023783.1 | — | 215604_x_at |
| BF218804 | AFURS1 | 212297_at |
| NM_001281.1 | CKAP1 | 201804_x_at |
| NM_024006.1 | IMAGE3455200 | 217949_s_at |
| AK023843.1 | PGF | 215179_x_at |
| BC001602.1 | CFLAR | 211316_x_at |
| BC034707.1 | — | 217653_x_at |
| BC064619.1 | CD24 | 266_s_at |
| AY280502.1 | EPHB6 | 204718_at |
| BC059387.1 | MYO1A | 211916_s_at |
| | — | 215032_at |
| AF135421.1 | GMPPB | 219920_s_at |
| BC061522.1 | MGC70907 | 211996_s_at |
| L76200.1 | GUK1 | 200075_s_at |
| U50532.1 | CG005 | 214753_at |
| BC006547.2 | EEF2 | 204102_s_at |
| BC008797.2 | FVT1 | 202419_at |
| BC000807.1 | ZNF160 | 214715_x_at |
| AL080112.1 | — | 216859_x_at |
| BC033718.1 /// | C21orf106 | 215529_x_at |
| BC046176.1 /// | | |
| BC038443.1 | | |
| NM_000346.1 | SOX9 | 202936_s_at |
| BC008710.1 | SUI1 | 212130_x_at |
| Hs.288575 | — | 215204_at |
| (Unigene ID) | | |
| AF020591.1 | AF020591 | 218735_s_at |
| BC000423.2 | ATP6V0B | 200078_s_at |
| BC002503.2 | SAT | 203455_s_at |
| BC008710.1 | SUI1 | 212227_x_at |
| | — | 222282_at |
| BC009185.2 | DCLRE1C | 219678_x_at |
| Hs.528304 | ADAM28 | 208268_at |
| (UNIGENE ID) | | |
| U50532.1 | CG005 | 221899_at |
| BC013923.2 | SOX2 | 213721_at |
| BC031091 | ODAG | 214718_at |
| NM_007062 | PWP1 | 201608_s_at |
| Hs.249591 | FLJ20686 | 205684_s_at |
| (Unigene ID) | | |
| BC075839.1 /// | KRT8 | 209008_x_at |
| BC073760.1 | | |
| BC072436.1 /// | HYOU1 | 200825_s_at |
| BC004560.2 | | |
| BC001016.2 | NDUFA8 | 218160_at |
| Hs.286261 | FLJ20195 | 57739_at |
| (Unigene ID) | | |
| AF348514.1 | — | 211921_x_at |
| BC005023.1 | CGI-128 | 218074_at |
| BC066337.1 /// | KTN1 | 200914_x_at |
| BC058736.1 /// | | |
| BC050555.1 | | |
| | — | 216384_x_at |
| Hs.216623 | ATP8B1 | 214594_x_at |
| (Unigene ID) | | |
| BC072400.1 | THOC2 | 222122_s_at |
| BC041073.1 | PRKX | 204060_s_at |
| U43965.1 | ANK3 | 215314_at |
| | — | 208238_x_at |
| BC021258.2 | TRIM5 | 210705_s_at |
| BC016057.1 | USH1C | 211184_s_at |
| BC016713.1 /// | PARVA | 215418_at |
| BC014535.1 /// | | |
| AF237771.1 | | |
| BC000360.2 | EIF4EL3 | 209393_s_at |
| BC007455.2 | SH3GLB1 | 210101_x_at |
| BC000701.2 | KIAA0676 | 212052_s_at |
| BC010067.2 | CHC1 | 215011_at |
| BC023528.2 /// | C14orf87 | 221932_s_at |
| BC047680.1 | | |
| BC064957.1 | KIAA0102 | 201239_s_at |
| Hs.156701 | — | 215553_x_at |
| (Unigene ID) | | |
| BC030619.2 | KIAA0779 | 213351_s_at |
| BC008710.1 | SUI1 | 202021_x_at |
| U43965.1 | ANK3 | 209442_x_at |
| BC066329.1 | SDHC | 210131_x_at |
| Hs.438867 | — | 217713_x_at |
| (Unigene ID) | | |
| BC035025.2 /// | ALMS1 | 214707_x_at |
| BC050330.1 | | |
| BC023976.2 | PDAP2 | 203272_s_at |
| BC074852.2 /// | PRKY | 206279_at |
| BC074851.2 | | |
| Hs.445885 | KIAA1217 | 214912_at |
| (Unigene ID) | | |
| BC008591.2 /// | KIAA0100 | 201729_s_at |
| BC050440.1 /// | | |
| BC048096.1 | | |
| AF365931.1 | ZNF264 | 205917_at |
| AF257099.1 | PTMA | 200772_x_at |
| BC028912.1 | DNAJB9 | 202842_s_at |

TABLE 6
Examples of biomarkers that may distinguish smokers
with lung cancer from smokers without lung cancer.

| GenBank ID | Gene Name | Affymetrix ID |
|---|---|---|
| NM_007062.1 | PWP1 | 201608_s_at |
| NM_001281.1 | CKAP1 | 201804_x_at |
| BC000120.1 | | 202355_s_at |
| NM_014255.1 | TMEM4 | 202857_at |
| BC002642.1 | CTSS | 202901_x_at |
| NM_000346.1 | SOX9 | 202936_s_at |
| NM_006545.1 | NPR2L | 203246_s_at |
| BG034328 | | 203588_s_at |
| NM_021822.1 | APOBEC3G | 204205_at |
| NM_021069.1 | ARGBP2 | 204288_s_at |
| NM_019067.1 | FLJ10613 | 205010_at |
| NM_017925.1 | FLJ20686 | 205684_s_at |
| NM_017932.1 | FLJ20700 | 207730_x_at |
| NM_030757.1 | MKRN4 | 208082_x_at |
| NM_030972.1 | MGC5384 | 208137_x_at |
| AF126181.1 | BCG1 | 208682_s_at |
| U93240.1 | | 209653_at |
| U90552.1 | | 209770_at |
| AF151056.1 | | 210434_x_at |
| U85430.1 | NFATC3 | 210556_at |
| U51007.1 | | 211609_x_at |
| BC005969.1 | | 211759_x_at |
| NM_002271.1 | | 211954_s_at |
| AL566172 | | 212041_at |
| AB014576.1 | KIAA0676 | 212052_s_at |
| BF218804 | AFURS1 | 212297_at |
| AK022494.1 | | 212932_at |
| AA114843 | | 213884_s_at |
| BE467941 | | 214153_at |
| NM_003541.1 | HIST1H4K | 214463_x_at |
| R83000 | BTF3 | 214800_x_at |
| AL161952.1 | GLUL | 215001_s_at |
| AK023843.1 | PGF | 215179_x_at |
| AK021571.1 | MUC20 | 215208_x_at |
| AK023783.1 | — | 215604_x_at |
| AU147182 | | 215620_at |
| AL080112.1 | — | 216859_x_at |
| AW971983 | | 217588_at |
| AI683552 | — | 217679_x_at |
| NM_024006.1 | IMAGE3455200 | 217949_s_at |
| AK026565.1 | FLJ10534 | 218155_x_at |
| NM_014182.1 | ORMDL2 | 218556_at |
| NM_021800.1 | DNAJC12 | 218976_at |
| NM_016049.1 | CGI-112 | 219203_at |
| NM_019023.1 | PRMT7 | 219408_at |
| NM_021971.1 | GMPPB | 219920_s_at |
| NM_014128.1 | — | 220856_x_at |
| AK025651.1 | | 221648_s_at |
| AA133341 | C14orf87 | 221932_s_at |
| AF198444.1 | | 222168_at |

TABLE 7
Examples of biomarkers that may distinguish smokers
having lung cancer from smokers without lung cancer.

| GenBank ID | Gene Name | Affymetrix ID |
|---|---|---|
| NM_007062.1 | PWP1 | 201608_s_at |
| NM_001281.1 | CKAP1 | 201804_x_at |
| BC002642.1 | CTSS | 202901_x_at |
| NM_000346.1 | SOX9 | 202936_s_at |
| NM_006545.1 | NPR2L | 203246_s_at |
| BG034328 | | 203588_s_at |
| NM_019067.1 | FLJ10613 | 205010_at |
| NM_017925.1 | FLJ20686 | 205684_s_at |
| NM_017932.1 | FLJ20700 | 207730_x_at |
| NM_030757.1 | MKRN4 | 208082_x_at |
| NM_030972.1 | MGC5384 | 208137_x_at |
| NM_002268 /// | KPNA4 | 209653_at |
| NM_032771 | | |
| NM_007048 /// | BTN3A1 | 209770_at |
| NM_194441 | | |
| NM_006694 | JBT | 210434_x_at |
| U85430.1 | NFATC3 | 210556_at |
| NM_004691 | ATP6V0D1 | 212041_at |
| AB014576.1 | KIAA0676 | 212052_s_at |
| BF218804 | AFURS1 | 212297_at |
| BE467941 | | 214153_at |
| R83000 | BTF3 | 214800_x_at |
| AL161952.1 | GLUL | 215001_s_at |
| AK023843.1 | PGF | 215179_x_at |
| AK021571.1 | MUC20 | 215208_x_at |
| AK023783.1 | — | 215604_x_at |
| AL080112.1 | — | 216859_x_at |
| AW971983 | | 217588_at |
| AI683552 | — | 217679_x_at |
| NM_024006.1 | IMAGE3455200 | 217949_s_at |
| AK026565.1 | FLJ10534 | 218155_x_at |
| NM_014182.1 | ORMDL2 | 218556_at |
| NM_021800.1 | DNAJC12 | 218976_at |
| NM_016049.1 | CGI-112 | 219203_at |
| NM_021971.1 | GMPPB | 219920_s_at |
| NM_014128.1 | — | 220856_x_at |
| AA133341 | C14orf87 | 221932_s_at |
| AF198444.1 | | 222168_at |

TABLE 8
Examples of biomarkers that may identify
a diagnosis or a prognosis of lung cancer.

| | Gene symbol |
|---|---|
| Affymetrix ID | (HUGO ID) |
| 200729_s_at | ACTR2 |
| 200760_s_at | ARL6IP5 |
| 201399_s_at | TRAM1 |
| 201444_s_at | ATP6AP2 |
| 201635_s_at | FXR1 |
| 201689_s_at | TPD52 |
| 201925_s_at | DAF |
| 201926_s_at | DAF |
| 201946_s_at | CCT2 |
| 202118_s_at | CPNE3 |
| 202704_at | TOB1 |
| 202833_s_at | SERPINA1 |
| 202935_s_at | SOX9 |
| 203413_at | NELL2 |
| 203881_s_at | DMD |
| 203908_at | SLC4A4 |
| 204006_s_at | FCGR3A /// FCGR3B |
| 204403_x_at | KIAA0738 |
| 204427_s_at | RNP24 |
| 206056_x_at | SPN |
| 206169_x_at | RoXaN |
| 207730_x_at | HDGF2 |
| 207756_at | — |
| 207791_s_at | RAB1A |
| 207953_at | AD7C-NTP |
| 208137_x_at | — |
| 208246_x_at | TK2 |
| 208654_s_at | CD164 |
| 208892_s_at | DUSP6 |
| 209189_at | FOS |
| 209204_at | LMO4 |
| 209267_s_at | SLC39A8 |
| 209369_at | ANXA3 |
| 209656_s_at | TMEM47 |
| 209774_x_at | CXCL2 |
| 210145_at | PLA2G4A |
| 210168_at | C6 |
| 210317_s_at | YWHAE |
| 210397_at | DEFB1 |
| 210679_x_at | — |
| 211506_s_at | IL8 |
| 212006_at | UBXD2 |
| 213089_at | LOC153561 |
| 213736_at | COX5B |
| 213813_x_at | — |
| 214007_s_at | PTK9 |
| 214146_s_at | PPBP |
| 214594_x_at | ATP8B1 |
| 214707_x_at | ALMS1 |
| 214715_x_at | ZNF160 |
| 215204_at | SENP6 |
| 215208_x_at | RPL35A |
| 215385_at | FTO |
| 215600_x_at | FBXW12 |
| 215604_x_at | UBE2D2 |
| 215609_at | STARD7 |
| 215628_x_at | PPP2CA |
| 215800_at | DUOX1 |
| 215907_at | BACH2 |
| 215978_x_at | LOC152719 |
| 216834_at | — |
| 216858_x_at | — |
| 217446_x_at | — |
| 217653_x_at | — |
| 217679_x_at | — |
| 217715_x_at | ZNF354A |
| 217826_s_at | UBE2J1 |
| 218155_x_at | FLJ10534 |
| 218976_at | DNAJC12 |
| 219392_x_at | FLJ11029 |
| 219678_x_at | DCLRE1C |
| 220199_s_at | FLJ12806 |
| 220389_at | FLJ23514 |
| 220720_x_at | FLJ14346 |
| 221191_at | DKFZP434A0131 |
| 221310_at | FGF14 |
| 221765_at | — |
| 222027_at | NUCKS |
| 222104_x_at | GTF2H3 |
| 222358_x_at | — |

TABLE 9
Examples of biomarkers that may identify
a diagnosis or a prognosis of lung cancer.

| Affymetrix ID | (HUGO ID) |
|---|---|
| 200729_s_at | ACTR2 |
| 200760_s_at | ARL6IP5 |
| 201399_s_at | TRAM1 |
| 201444_s_at | ATP6AP2 |
| 201635_s_at | FXR1 |
| 201689_s_at | TPD52 |
| 201925_s_at | DAF |
| 201926_s_at | DAF |
| 201946_s_at | CCT2 |
| 202118_s_at | CPNE3 |
| 202704_at | TOB1 |
| 202833_s_at | SERPINA1 |
| 202935_s_at | SOX9 |
| 203413_at | NELL2 |
| 203881_s_at | DMD |
| 203908_at | SLC4A4 |
| 204006_s_at | FCGR3A /// FCGR3B |
| 204403_x_at | KIAA0738 |
| 204427_s_at | RNP24 |
| 206056_x_at | SPN |
| 206169_x_at | RoXaN |
| 207730_x_at | HDGF2 |
| 207756_at | — |
| 207791_s_at | RAB1A |
| 207953_at | AD7C-NTP |
| 208137_x_at | — |
| 208246_x_at | TK2 |
| 208654_s_at | CD164 |
| 208892_s_at | DUSP6 |
| 209189_at | FOS |
| 209204_at | LMO4 |
| 209267_s_at | SLC39A8 |
| 209369_at | ANXA3 |
| 209656_s_at | TMEM47 |
| 209774_x_at | CXCL2 |
| 210145_at | PLA2G4A |
| 210168_at | C6 |
| 210317_s_at | YWHAE |
| 210397_at | DEFB1 |
| 210679_x_at | — |
| 211506_s_at | IL8 |
| 212006_at | UBXD2 |
| 213089_at | LOC153561 |
| 213736_at | COX5B |
| 213813_x_at | — |
| 214007_s_at | PTK9 |
| 214146_s_at | PPBP |
| 214594_x_at | ATP8B1 |
| 214707_x_at | ALMS1 |
| 214715_x_at | ZNF160 |
| 215204_at | SENP6 |
| 215208_x_at | RPL35A |
| 215385_at | FTO |
| 215600_x_at | FBXW12 |
| 215604_x_at | UBE2D2 |
| 215609_at | STARD7 |
| 215628_x_at | PPP2CA |
| 215800_at | DUOX1 |
| 215907_at | BACH2 |
| 215978_x_at | LOC152719 |
| 216834_at | — |
| 216858_x_at | — |
| 217446_x_at | — |
| 217653_x_at | — |
| 217679_x_at | — |
| 217715_x_at | ZNF354A |
| 217826_s_at | UBE2J1 |
| 218155_x_at | FLJ10534 |
| 218976_at | DNAJC12 |
| 219392_x_at | FLJ11029 |
| 219678_x_at | DCLRE1C |
| 220199_s_at | FLJ12806 |
| 220389_at | FLJ23514 |
| 220720_x_at | FLJ14346 |
| 221191_at | DKFZP434A0131 |
| 221310_at | FGF14 |
| 221765_at | — |
| 222027_at | NUCKS |
| 222104_x_at | GTF2H3 |
| 222358_x_at | — |
| 202113_s_at | SNX2 |
| 207133_x_at | ALPK1 |
| 218989_x_at | SLC30A5 |
| 200751_s_at | HNRPC |
| 220796_x_at | SLC35E1 |
| 209362_at | SURB7 |
| 216248_s_at | NR4A2 |
| 203138_at | HAT1 |
| 221428_s_at | TBL1XR1 |
| 218172_s_at | DERL1 |
| 215861_at | FLJ14031 |
| 209288_s_at | CDC42EP3 |
| 214001_x_at | RPS10 |
| 209116_x_at | HBB |
| 215595_x_at | GCNT2 |
| 208891_at | DUSP6 |
| 215067_x_at | PRDX2 |
| 202918_s_at | PREI3 |
| 211985_s_at | CALM1 |
| 212019_at | RSL1D1 |
| 216187_x_at | KNS2 |
| 215066_at | PTPRF |
| 212192_at | KCTD12 |
| 217586_x_at | — |
| 203582_s_at | RAB4A |
| 220113_x_at | POLR1B |
| 217232_x_at | HBB |
| 201041_s_at | DUSP1 |
| 211450_s_at | MSH6 |
| 202648_at | RPS19 |
| 202936_s_at | SOX9 |
| 204426_at | RNP24 |
| 206392_s_at | RARRES1 |
| 208750_s_at | ARF1 |
| 202089_s_at | SLC39A6 |
| 211297_s_at | CDK7 |
| 215373_x_at | FLJ12151 |
| 213679_at | FLJ13946 |
| 201694_s_at | EGR1 |
| 209142_s_at | UBE2G1 |
| 217706_at | LOC220074 |
| 212991_at | FBX09 |
| 201289_at | CYR61 |
| 206548_at | FLJ23556 |
| 202593_s_at | MIR16 |
| 202932_at | YES1 |
| 220575_at | FLJ11800 |
| 217713_x_at | DKFZP566N034 |
| 211953_s_at | RANBP5 |
| 203827_at | WIPI49 |
| 221997_s_at | MRPL52 |
| 217662_x_at | BCAP29 |
| 218519_at | SLC35A5 |
| 214833_at | KIAA0792 |
| 201339_s_at | SCP2 |
| 203799_at | CD302 |
| 211090_s_at | PRPF4B |
| 220071_x_at | C15orf25 |
| 203946_s_at | ARG2 |
| 213544_at | ING1L |
| 209908_s_at | — |
| 201688_s_at | TPD52 |
| 215587_x_at | BTBD14B |
| 201699_at | PSMC6 |
| 214902_x_at | FLJ42393 |
| 214041_x_at | RPL37A |
| 203987_at | FZD6 |
| 211696_x_at | HBB |
| 218025_s_at | PECI |
| 215852_x_at | KIAA0889 |
| 209458_x_at | HBA1 /// HBA2 |
| 219410_at | TMEM45A |
| 215375_x_at | — |
| 206302_s_at | NUDT4 |
| 208783_s_at | MCP |
| 211374_x_at | — |
| 220352_x_at | MGC4278 |
| 216609_at | TXN |
| 201942_s_at | CPD |
| 202672_s_at | ATF3 |
| 204959_at | MNDA |
| 211996_s_at | KIAA0220 |
| 222035_s_at | PAPOLA |
| 208808_s_at | HMGB2 |
| 203711_s_at | HIBCH |
| 215179_x_at | PGF |
| 213562_s_at | SQLE |
| 203765_at | GCA |
| 214414_x_at | HBA2 |
| 217497_at | ECGF1 |
| 220924_s_at | SLC38A2 |
| 218139_s_at | C14orf108 |
| 201096_s_at | ARF4 |
| 220361_at | FLJ12476 |
| 202169_s_at | AASDHPPT |
| 202527_s_at | SMAD4 |
| 202166_s_at | PPP1R2 |
| 204634_at | NEK4 |
| 215504_x_at | — |
| 202388_at | RGS2 |
| 215553_x_at | WDR45 |
| 200598_s_at | TRA1 |
| 202435_s_at | CYP1B1 |
| 216206_x_at | MAP2K7 |
| 212582_at | OSBPL8 |
| 216509_x_at | MLLT10 |
| 200908_s_at | RPLP2 |
| 215108_x_at | TNRC9 |
| 213872_at | C6orf62 |
| 214395_x_at | EEF1D |
| 222156_x_at | CCPG1 |
| 201426_s_at | VIM |
| 221972_s_at | Cab45 |
| 219957_at | — |
| 215123_at | — |
| 212515_s_at | DDX3X |
| 203357_s_at | CAPN7 |
| 211711_s_at | PTEN |
| 206165_s_at | CLCA2 |
| 213959_s_at | KIAA1005 |
| 215083_at | PSPC1 |
| 219630_at | PDZK1IP1 |
| 204018_x_at | HBA1 /// HBA2 |
| 208671_at | TDE2 |
| 203427_at | ASF1A |
| 215281_x_at | POGZ |
| 205749_at | CYP1A1 |
| 212585_at | OSBPL8 |
| 211745_x_at | HBA1 /// HBA2 |
| 208078_s_at | SNF1LK |
| 218041_x_at | SLC38A2 |
| 212588_at | PTPRC |
| 212397_at | RDX |
| 208268_at | ADAM28 |
| 207194_s_at | ICAM4 |
| 222252_x_at | — |
| 217414_x_at | HBA2 |
| 207078_at | MED6 |
| 215268_at | KIAA0754 |
| 221387_at | GPR147 |
| 201337_s_at | VAMP3 |
| 220218_at | C9orf68 |
| 222356_at | TBL1Y |
| 208579_x_at | H2BFS |
| 219161_s_at | CKLF |
| 202917_s_at | S100A8 |
| 204455_at | DST |
| 211672_s_at | ARPC4 |
| 201132_at | HNRPH2 |
| 218313_s_at | GALNT7 |
| 218930_s_at | FLJ11273 |
| 219166_at | Cl4orf104 |
| 212805_at | KIAA0367 |
| 201551_s_at | LAMP1 |
| 202599_s_at | NRIP1 |
| 203403_s_at | RNF6 |
| 214261_s_at | ADH6 |
| 202033_s_at | RB1CC1 |
| 203896_s_at | PLCB4 |
| 209703_x_at | DKFZP586A0522 |
| 211699_x_at | HBA1 /// HBA2 |
| 210764_s_at | CYR61 |
| 206391_at | RARRES1 |
| 201312_s_at | SH3BGRL |
| 200798_x_at | MCL1 |
| 214912_at | — |
| 20462l_s_at | NR4A2 |
| 217761_at | MTCBP-1 |
| 205830_at | CLGN |
| 218438_s_at | MED28 |
| 207475_at | FABP2 |
| 208621_s_at | VIL2 |
| 202436_s_at | CYP1B1 |
| 202539_s_at | HMGCR |
| 210830_s_at | PON2 |
| 211906_s_at | SERPINB4 |
| 202241_at | TRIB1 |
| 203594_at | RTCD1 |
| 215863_at | TFR2 |
| 221992_at | LOC283970 |
| 221872_at | RARRES1 |
| 219564_at | KCNJ16 |
| 201329_s_at | ETS2 |
| 214188_at | HIS1 |
| 201667_at | GJA1 |
| 201464_x_at | JUN |
| 215409_at | LOC254531 |
| 202583_s_at | RANBP9 |
| 215594_at | — |
| 214326_x_at | JUND |
| 217140_s_at | VDAC1 |
| 215599_at | SMA4 |
| 209896_s_at | PTPN11 |
| 204846_at | CP |
| 222303_at | — |
| 218218_at | DIP13B |
| 211015_s_at | HSPA4 |
| 208666_s_at | ST13 |
| 203191_at | ABCB6 |
| 202731_at | PDCD4 |
| 209027_s_at | ABI1 |
| 205979_at | SCGB2A1 |
| 21635l_x_at | DAZ1 /// DAZ3 /// |
| | DAZ2 /// DAZ4 |
| 220240_s_at | C13orf11 |
| 204482_at | CLDN5 |
| 217234_s_at | VIL2 |
| 214350_at | SNTB2 |
| 201693_s_at | EGR1 |
| 212328_at | KIAA1102 |
| 220168_at | CASC1 |
| 203628_at | IGF1R |
| 204622_x_at | NR4A2 |
| 213246_at | C14orf109 |
| 218728_s_at | HSPC163 |
| 214753_at | PFAAP5 |
| 206336_at | CXCL6 |
| 201445_at | CNN3 |
| 209886_s_at | SMAD6 |
| 213376_at | ZBTB1 |
| 213887_s_at | POLR2E |
| 204783_at | MLF1 |
| 218824_at | FLJ10781 |
| 212417_at | SCAMP1 |
| 202437_s_at | CYP1B1 |
| 217528_at | CLCA2 |
| 218170_at | ISOC1 |
| 206278_at | PTAFR |
| 201939_at | PLK2 |
| 200907_s_at | KIAA0992 |
| 207480_s_at | MEIS2 |
| 201417_at | SOX4 |
| 213826_s_at | — |
| 214953_s_at | APP |
| 204897_at | PTGER4 |
| 201711_x_at | RANBP2 |
| 202457_s_at | PPP3CA |
| 206683_at | ZNF165 |
| 214581_x_at | TNFRSF21 |
| 203392_s_at | CTBP1 |
| 212720_at | PAPOLA |
| 207758_at | PPM1F |
| 220995_at | STXBP6 |
| 213831_at | HLA-DQA1 |
| 212044_s_at | — |
| 202434_s_at | CYP1B1 |
| 206166_s_at | CLCA2 |
| 218343_s_at | GTF3C3 |
| 202557_at | STCH |
| 201133_s_at | PJA2 |
| 213605_s_at | MGC22265 |
| 210947_s_at | MSH3 |
| 208310_s_at | C7orf28A /// C7orf28B |
| 209307_at | — |
| 215387_x_at | GPC6 |
| 213705_at | MAT2A |
| 213979_s_at | — |
| 212731_at | LOC157567 |
| 210117_at | SPAG1 |
| 200641_s_at | YWHAZ |
| 210701_at | CFDP1 |
| 217152_at | NCOR1 |
| 204224_s_at | GCH1 |
| 202028_s_at | — |
| 201735_s_at | CLCN3 |
| 208447_s_at | PRPS1 |
| 220926_s_at | C1orf22 |
| 211505_s_at | STAU |
| 221684_s_at | NYX |
| 206906_at | ICAM5 |
| 213228_at | PDE8B |
| 217202_s_at | GLUL |
| 211713_x_at | KIAA0101 |
| 215012_at | ZNF451 |
| 200806_s_at | HSPD1 |
| 201466_s_at | JUN |
| 211564_s_at | PDLIM4 |
| 207850_at | CXCL3 |
| 221841_s_at | KLF4 |
| 200605_s_at | PRKAR1A |
| 221198_at | SCT |
| 201772_at | AZIN1 |
| 205009_at | TFF1 |
| 205542_at | STEAP1 |
| 218195_at | C6orf211 |
| 213642_at | — |
| 212891_s_at | GADD45GIP1 |
| 202798_at | SEC24B |
| 222207_x_at | — |
| 202638_s_at | ICAM1 |
| 200730_s_at | PTP4A1 |
| 219355_at | FLJ10178 |
| 220266_s_at | KLF4 |
| 201259_s_at | SYPL |
| 209649_at | STAM2 |
| 220094_s_at | C6orf79 |
| 221751_at | PANK3 |
| 200008_s_at | GDI2 |
| 205078_at | PIGF |
| 218842_at | FLJ21908 |
| 202536_at | CHMP2B |
| 220184_at | NANOG |
| 201117_s_at | CPE |
| 219787_s_at | ECT2 |
| 206628_at | SLC5A1 |
| 204007_at | FCGR3B |
| 209446_s_at | — |
| 211612_s_at | IL13RA1 |
| 220992_s_at | C1orf25 |
| 221899_at | PFAAP5 |
| 221719_s_at | LZTS1 |
| 201473_at | JUNB |
| 221193_s_at | ZCCHC10 |
| 215659_at | GSDML |
| 205157_s_at | KRT17 |
| 201001_s_at | UBE2V1 /// Kua-UEV |
| 216789_at | — |
| 205506_at | VIL1 |
| 204875_s_at | GMDS |
| 207191_s_at | ISLR |
| 202779_s_at | UBE2S |
| 210370_s_at | LY9 |
| 202842_s_at | DNAJB9 |
| 201082_s_at | DCTN1 |
| 215588_x_at | RIOK3 |
| 211076_x_at | DRPLA |
| 210230_at | — |
| 206544_x_at | SMARCA2 |
| 208852_s_at | CANX |
| 215405_at | MYO1E |
| 208653_s_at | CD164 |
| 206355_at | GNAL |
| 210793_s_at | NUP98 |
| 215070_x_at | RABGAP1 |
| 203007_x_at | LYPLA1 |
| 203841_x_at | MAPRE3 |
| 206759_at | FCER2 |
| 202232_s_at | GA17 |
| 215892_at | — |
| 214359_s_at | HSPCB |
| 215810_x_at | DST |
| 208937_s_at | ID1 |
| 213664_at | SLC1A1 |
| 219338_s_at | FLJ20156 |
| 206595_at | CST6 |
| 207300_s_at | F7 |
| 213792_s_at | INSR |
| 209674_at | CRY1 |
| 40665_at | FNO3 |
| 217975_at | WBP5 |
| 210296_s_at | PXMP3 |
| 215483_at | AKAP9 |
| 212633_at | KIAA0776 |
| 206164_at | CLCA2 |
| 216813_at | — |
| 208925_at | C3orf4 |
| 219469_at | DNCH2 |
| 206016_at | CXorf37 |
| 216745_x_at | LRCH1 |
| 212999_x_at | HLA-DQB1 |
| 216859_x_at | — |
| 201636_at | — |
| 204272_at | LGALS4 |
| 215454_x_at | SFTPC |
| 215972_at | — |
| 220593_s_at | FLJ20753 |
| 222009_at | CGI-14 |
| 207115_x_at | MBTD1 |
| 216922_x_at | DAZ1 /// DAZ3 /// |
| | DAZ2 /// DAZ4 |
| 217626_at | AKR1C1 /// AKR1C2 |
| 211429_s_at | SERPINA1 |
| 209662_at | CETN3 |
| 201629_s_at | ACP1 |
| 201236_s_at | BTG2 |
| 217137_x_at | — |
| 212476_at | CENTB2 |
| 218545_at | FLJ11088 |
| 208857_s_at | PCMT1 |
| 221931_s_at | SEH1L |
| 215046_at | FLJ23861 |
| 220222_at | PRO1905 |
| 209737_at | AIP1 |
| 203949_at | MPO |
| 219290_x_at | DAPP1 |
| 205116_at | LAMA2 |
| 222316_at | VDP |
| 203574_at | NFIL3 |
| 207820_at | ADH1A |
| 20375l_x_at | JUND |
| 202930_s_at | SUCLA2 |
| 215404_x_at | FGFR1 |
| 216266_s_at | ARFGEF1 |
| 212806_at | KIAA0367 |
| 219253_at | — |
| 214605_x_at | GPR1 |
| 205403_at | IL1R2 |
| 222282_at | PAPD4 |
| 214129_at | PDE4DIP |
| 209259_s_at | CSPG6 |
| 216900_s_at | CHRNA4 |
| 221943_x_at | RPL38 |
| 215386_at | AUTS2 |
| 201990_s_at | CREBL2 |
| 220145_at | FLJ21159 |
| 221173_at | USH1C |
| 214900_at | ZKSCAN1 |
| 203290_at | HLA-DQA1 |
| 215382_x_at | TPSAB1 |
| 201631_s_at | IER3 |
| 212188_at | KCTD12 |
| 220428_at | CD207 |
| 215349_at | — |
| 213928_s_at | HRB |
| 221228_s_at | — |
| 202069_s_at | IDH3A |
| 208554_at | POU4F3 |
| 209504_s_at | PLEKHB1 |
| 212989_at | TMEM23 |
| 216197_at | ATF7IP |
| 204748_at | PTGS2 |
| 205221_at | HGD |
| 214705_at | INADL |
| 213939_s_at | RIPX |
| 203691_at | PI3 |
| 220532_s_at | LR8 |
| 209829_at | C6orf32 |
| 206515_at | CYP4F3 |
| 218541_s_at | C8orf4 |
| 210732_s_at | LGALS8 |
| 202643_s_at | TNFAIP3 |
| 218963_s_at | KRT23 |
| 213304_at | KIAA0423 |
| 202768_at | FOSB |
| 205623_at | ALDH3A1 |
| 206488_s_at | CD36 |
| 204319_s_at | RGS10 |
| 217811_at | SELT |
| 202746_at | ITM2A |
| 221127_s_at | RIG |
| 209821_at | C9orf26 |
| 220957_at | CTAGE1 |
| 215577_at | UBE2E1 |
| 214731_at | DKFZp547A023 |
| 210512_s_at | VEGF |
| 205267_at | POU2AF1 |
| 216202_s_at | SPTLC2 |
| 220477_s_at | C20orf30 |
| 205863_at | S100A12 |
| 215780_s_at | SET /// LOC389168 |
| 218197_s_at | OXR1 |
| 203077_s_at | SMAD2 |
| 222339_x_at | — |
| 200698_at | KDELR2 |
| 210540_s_at | B4GALT4 |
| 217725_x_at | PAI-RBP1 |
| 217082_at | — |

TABLE 10
Examples of biomarkers that may identify a diagnosis or prognosis
of lung cancer.

| Affymetrix ID | HUGO ID |
|---|---|
| 207953_at | AD7C-NTP |
| 215208_x_at | RPL35A |
| 215604_x_at | UBE2D2 |
| 218155_x_at | FLJ10534 |
| 216858_x_at | — |
| 208137_x_at | — |
| 214715_x_at | ZNF160 |
| 217715_x_at | ZNF354A |
| 220720_x_at | FLJ14346 |
| 215907_at | BACH2 |
| 217679_x_at | — |
| 206169_x_at | RoXaN |
| 208246_x_at | TK2 |
| 222104_x_at | GTF2H3 |
| 206056_x_at | SPN |
| 217653_x_at | — |
| 210679_x_at | — |
| 207730_x_at | HDGF2 |
| 214594_x_at | ATP8B1 |

TABLE 11
Examples of biomarkers that may identify a relationship between
expression profiles of epithelial cells in the bronchus and upper
airways in response to smoke.

| AffyID | GeneName (HUGO ID) |
|---|---|
| 202437_s_at | CYP1B1 |
| 206561_s_at | AKR1B10 |
| 202436_s_at | CYP1B1 |
| 205749_at | CYP1A1 |
| 202435_s_at | CYP1B1 |
| 201884_at | CEACAM5 |
| 205623_at | ALDH3A1 |
| 217626_at | — |
| 209921_at | SLC7A11 |
| 209699_x_at | AKR1C2 |
| 201467_s_at | NQO1 |
| 201468_s_at | NQO1 |
| 202831_at | GPX2 |
| 214303_x_at | MUC5AC |
| 211653_x_at | AKR1C2 |
| 214385_s_at | MUC5AC |
| 216594_x_at | AKR1C1 |
| 205328_at | CLDN10 |
| 209160_at | AKR1C3 |
| 210519_s_at | NQO1 |
| 217678_at | SLC7A11 |
| 205221_at | HGD///LOC642252 |
| 204151_x_at | AKR1C1 |
| 207469_s_at | PIR |
| 206153_at | CYP4F11 |
| 205513_at | TCN1 |
| 209386_at | TM4SF1 |
| 209351_at | KRT14 |
| 204059_s_at | ME1 |
| 209213_at | CBR1 |
| 210505_at | ADH7 |
| 214404_x_at | SPDEF |
| 204058_at | ME1 |
| 218002_s_at | CXCL14 |
| 205499_at | SRPX2 |
| 210065_s_at | UPK1B |
| 204341_at | TRIM16///TRIM16L/// |
| | LOC653524 |
| 221841_s_at | KLF4 |
| 208864_s_at | TXN |
| 208699_x_at | TKT |
| 210397_at | DEFB1 |
| 204971_at | CSTA |
| 211657_at | CEACAM6 |
| 201463_s_at | TALDO1 |
| 214164_x_at | CA12 |
| 203925_at | GCLM |
| 201118_at | PGD |
| 201266_at | TXNRD1 |
| 203757_s_at | CEACAM6 |
| 202923_s_at | GCLC |
| 214858_at | GPC1 |
| 205009_at | TFF1 |
| 219928_s_at | CABYR |
| 203963_at | CA12 |
| 210064_s_at | UPK1B |
| 219956_at | GALNT6 |
| 208700_s_at | TKT |
| 203824_at | TSPAN8 |
| 207126_x_at | UGT1A10///UGT1A8/// |
| | UGT1A7/// |
| | UGT1A6///UGT1A |
| 213441_x_at | SPDEF |
| 207430_s_at | MSMB |
| 209369_at | ANXA3 |
| 217187_at | MUC5AC |
| 209101_at | CTGF |
| 212221_x_at | IDS |
| 215867_x_at | CA12 |
| 214211_at | FTH1 |
| 217755_at | HN1 |
| 201431_s_at | DPYSL3 |
| 204875_s_at | GMDS |
| 215125_s_at | UGT1A10///UGT1A8/// |
| | UGT1A7/// |
| | UGT1A6///UGT1A |
| 63825_at | ABHD2 |
| 202922_at | GCLC |
| 218313_s_at | GALNT7 |
| 210297_s_at | MSMB |
| 209448_at | HTATIP2 |
| 204532_x_at | UGT1A10///UGT1A8/// |
| | UGT1A7/// |
| | UGT1A6///UGT1A |
| 200872_at | S100A10 |
| 216351_x_at | DAZ1///DAZ3///DAZ2/// |
| | DAZ4 |
| 212223_at | IDS |
| 208680_at | PRDX1 |
| 206515_at | CYP4F3 |
| 208596_s_at | UGT1A10///UGT1A8/// |
| | UGT1A7/// |
| | UGT1A6//UGT1A |
| 209173_at | AGR2 |
| 204351_at | S100P |
| 202785_at | NDUFA7 |
| 204970_s_at | MAFG |
| 222016_s_at | ZNF323 |
| 200615_s_at | AP2B1 |
| 206094_x_at | UGT1A6 |
| 209706_at | NKX3-1 |
| 217977_at | SEPX1 |
| 201487_at | CTSC |
| 219508_at | GCNT3 |
| 204237_at | GULP1 |
| 213455_at | LOC283677 |
| 213624_at | SMPDL3A |
| 206770_s_at | SLC35A3 |
| 217975_at | WBP5 |
| 201263_at | TARS |
| 218696_at | EIF2AK3 |
| 212560_at | C11orf32 |
| 218885_s_at | GALNT12 |
| 212326_at | VPS13D |
| 217955_at | BCL2L13 |
| 203126_at | IMPA2 |
| 214106_s_at | GMDS |
| 209309_at | AZGP1 |
| 205112_at | PLCE1 |
| 215363_x_at | FOLH1 |
| 206302_s_at | NUDT4///NUDT4P1 |
| 200916_at | TAGLN2 |
| 205042_at | GNE |
| 217979_at | TSPAN13 |
| 203397_s_at | GALNT3 |
| 209786_at | HMGN4 |
| 211733_x_at | SCP2 |
| 207222_at | PLA2G10 |
| 204235_s_at | GULP1 |
| 205726_at | DIAPH2 |
| 203911_at | RAP1GAP |
| 200748_s_at | FTH1 |
| 212449_s_at | LYPLA1 |
| 213059_at | CREB3L1 |
| 201272_at | AKR1B1 |
| 208731_at | RAB2 |
| 205979_at | SCGB2A1 |
| 212805_at | KIAA0367 |
| 202804_at | ABCC1 |
| 218095_s_at | TPARL |
| 205566_at | ABHD2 |
| 209114_at | TSPAN1 |
| 202481_at | DHRS3 |
| 202805_s_at | ABCC1 |
| 219117_s_at | FKBP11 |
| 213172_at | TTC9 |
| 202554_s_at | GSTM3 |
| 218677_at | S100A14 |
| 203306_s_at | SLC35A1 |
| 204076_at | ENTPD4 |
| 200654_at | P4HB |
| 204500_s_at | AGTPBP1 |
| 208918_s_at | NADK |
| 221485_at | B4GALT5 |
| 221511_x_at | CCPG1 |
| 200733_s_at | PTP4A1 |
| 217901_at | DSG2 |
| 202769_at | CCNG2 |
| 202119_s_at | CPNE3 |
| 200945_s_at | SEC31L1 |
| 200924_s_at | SLC3A2 |
| 208736_at | ARPC3 |
| 221556_at | CDC14B |
| 221041_s_at | SLC17A5 |
| 215071_s_at | HIST1H2AC |
| 209682_at | CBLB |
| 209806_at | HIST1H2BK |
| 204485_s_at | TOM1L1 |
| 201666_at | TIMP1 |
| 203192_at | ABCB6 |
| 202722_s_at | GFPT1 |
| 213135_at | TIAM1 |
| 203509_at | SORL1 |
| 214620_x_at | PAM |
| 208919_s_at | NADK |
| 212724_at | RND3 |
| 212160_at | XPOT |
| 212812_at | SERINC5 |
| 200696_s_at | GSN |
| 217845_x_at | HIGD1A |
| 208612_at | PDIA3 |
| 219288_at | C3orf14 |
| 201923_at | PRDX4 |
| 211960_s_at | RAB7 |
| 64942_at | GPR153 |
| 201659_s_at | ARL1 |
| 202439_s_at | IDS |
| 209249_s_at | GHITM |
| 218723_s_at | RGC32 |
| 200087_s_at | TMED2 |
| 209694_at | PTS |
| 202320_at | GTF3C1 |
| 201193_at | IDH1 |
| 212233_at | — |
| 213891_s_at | — |
| 203041_s_at | LAMP2 |
| 202666_s_at | ACTL6A |
| 200863_s_at | RAB11A |
| 203663_s_at | COX5A |
| 211404_s_at | APLP2 |
| 201745_at | PTK9 |
| 217823_s_at | UBE2J1 |
| 202286_s_at | TACSTD2 |
| 212296_at | PSMD14 |
| 211048_s_at | PDIA4 |
| 214429_at | MTMR6 |
| 219429_at | FA2H |
| 212181_s_at | NUDT4 |
| 222116_s_at | TBC1D16 |
| 221689_s_at | PIGP |
| 209479_at | CCDC28A |
| 218434_s_at | AACS |
| 214665_s_at | CHP |
| 202085_at | TJP2 |
| 217992_s_at | EFHD2 |
| 203162_s_at | KATNB1 |
| 205406_s_at | SPA17 |
| 203476_at | TPBG |
| 201724_s_at | GALNT1 |
| 200599_s_at | HSP90B1 |
| 200929_at | TMED10 |
| 200642_at | SOD1 |
| 208946_s_at | BECN1 |
| 202562_s_at | C14orf1 |
| 201098_at | COPB2 |
| 221253_s_at | TXNDC5 |
| 201004_at | SSR4 |
| 203221_at | TLE1 |
| 201588_at | TXNL1 |
| 218684_at | LRRC8D |
| 208799_at | PSMB5 |
| 201471_s_at | SQSTM1 |
| 204034_at | ETHE1 |
| 208689_s_at | RPN2 |
| 212665_at | TIPARP |
| 200625_s_at | CAP1 |
| 213220_at | LOC92482 |
| 200709_at | FKBP1A |
| 203279_at | EDEM1 |
| 200068_s_at | CANX |
| 200620_at | TMEM59 |
| 200075_s_at | GUK1 |
| 209679_s_at | LOC57228 |
| 210715_s_at | SPINT2 |
| 209020_at | C20orf111 |
| 208091_s_at | ECOP |
| 200048_s_at | JTB |
| 218194_at | REXO2 |
| 209103_s_at | UFD1L |
| 208718_at | DDX17 |
| 219241_x_at | SSH3 |
| 216210_x_at | TRIOBP |
| 50277_at | GGA1 |
| 218023_s_at | FAM53C |
| 32540_at | PPP3CC |
| 43511_s_at | — |
| 212001_at | SFRS14 |
| 208637_x_at | ACTN1 |
| 201997_s_at | SPEN |
| 205073_at | CYP2J2 |
| 40837_at | TLE2 |
| 204447_at | ProSAPiP1 |
| 204604_at | PFTK1 |
| 210273_at | PCDH7 |
| 208614_s_at | FLNB |
| 206510_at | SIX2 |
| 200675_at | CD81 |
| 219228_at | ZNF331 |
| 209426_s_at | AMACR |
| 204000_at | GNB5 |
| 221742_at | CUGBP1 |
| 208883_at | EDD1 |
| 210166_at | TLR5 |
| 211026_s_at | MGLL |
| 220446_s_at | CHST4 |
| 207636_at | SERPINI2 |
| 212226_s_at | PPAP2B |
| 210347_s_at | BCL11A |
| 218424_s_at | STEAP3 |
| 204287_at | SYNGR1 |
| 205489_at | CRYM |
| 36129_at | RUTBC1 |
| 215418_at | PARVA |
| 213029_at | NFIB |
| 221016_s_at | TCF7L1 |
| 209737_at | MAGI2 |
| 220389_at | CCDC81 |
| 213622_at | COL9A2 |
| 204740_at | CNKSR1 |
| 212126_at | — |
| 207760_s_at | NCOR2 |
| 205258_at | INHBB |
| 213169_at | — |
| 33760_at | PEX14 |
| 220968_s_at | TSPAN9 |
| 221792_at | RAB6B |
| 205752_s_at | GSTM5 |
| 218974_at | FLJ10159 |
| 221748_s_at | TNS1 |
| 212185_x_at | MT2A |
| 209500_x_at | TNFSF13///TNFSF12-TNFSF13 |
| 215445_x_at | 1-Mar |
| 220625_s_at | ELF5 |
| 32137_at | JAG2 |
| 219747_at | FLJ23191 |
| 201397_at | PHGDH |
| 207913_at | CYP2F1 |
| 217853_at | TNS3 |
| 1598_g_at | GAS6 |
| 203799_at | CD302 |
| 203329_at | PTPRM |
| 208712_at | CCND1 |
| 210314_x_at | TNFSF13///TNFSF12-TNFSF13 |
| 213217_at | ADCY2 |
| 200953_s_at | CCND2 |
| 204326_x_at | MT1X |
| 213488_at | SNED1 |
| 213505_s_at | SFRS14 |
| 200982_s_at | ANXA6 |
| 211732_x_at | HNMT |
| 202587_s_at | AK1 |
| 396_f_at | EPOR |
| 200878_at | EPAS1 |
| 213228_at | PDE8B |
| 215785_s_at | CYFIP2 |
| 213601_at | SLIT1 |
| 37953_s_at | ACCN2 |
| 205206_at | KALI |
| 212859_x_at | MT1E |
| 217165_x_at | MT1F |
| 204754_at | HLF |
| 218225_at | SITPEC |
| 209784_s_at | JAG2 |
| 211538_s_at | HSPA2 |
| 211456_x_at | LOC650610 |
| 204734_at | KRT15 |
| 201563_at | SORD |
| 202746_at | ITM2A |
| 218025_s_at | PECI |
| 203914_x_at | HPGD |
| 200884_at | CKB |
| 204753_s_at | HLF |
| 207718_x_at | CYP2A6///CYP2A7/// |
| | CYP2A7P1///CYP2A13 |
| 218820_at | C14orf132 |
| 204745_x_at | MT1G |
| 204379_s_at | FGFR3 |
| 207808_s_at | PROS1 |
| 207547_s_at | FAM107A |
| 208581_x_at | MT1X |
| 205384_at | FXYD1 |
| 213629_x_at | MT1F |
| 823_at | CX3CL1 |
| 203687_at | CX3CL1 |
| 211295_x_at | CYP2A6 |
| 204755_x_at | HLF |
| 209897_s_at | SLIT2 |
| 40093_at | BCAM |
| 211726_s_at | FMO2 |
| 206461_x_at | MT1H |
| 219250_s_at | FLRT3 |
| 210524_x_at | — |
| 220798_x_at | PRG2 |
| 219410_at | TMEM45A |
| 205680_at | MMP10 |
| 217767_at | C3///LOC653879 |
| 220562_at | CYP2W1 |
| 210445_at | FABP6 |
| 205725_at | SCGB1A1 |
| 213432_at | MUC5B///LOC649768 |
| 209074_s_at | FAM107A |
| 216346_at | SEC14L3 |

TABLE 12
Examples of biomarkers that may be differentially expressed in
bronchial epithelial genes among genes highly changed in a nasal
epithelium in response to smoking.

| AffxID | Hugo ID |
|---|---|
| 203369_x_at | — |
| 218434_s_at | AACS |
| 205566_at | ABHD2 |
| 217687_at | ADCY2 |
| 210505_at | ADH7 |
| 205623_at | ALDH3A1 |
| 200615_s_at | AP2B1 |
| 214875_x_at | APLP2 |
| 212724_at | ARHE |
| 201659_s_at | ARL1 |
| 208736_at | ARPC3 |
| 213624_at | ASM3A |
| 209309_at | AZGP1 |
| 217188_s_at | C14orf1 |
| 200620_at | C1orf8 |
| 200068_s_at | CANX |
| 213798_s_at | CAP1 |
| 200951_s_at | CCND2 |
| 202769_at | CCNG2 |
| 201884_at | CEACAM5 |
| 203757_s_at | CEACAM6 |
| 214665_s_at | CHP |
| 205328_at | CLDN10 |
| 203663_s_at | COX5A |
| 202119_s_at | CPNE3 |
| 221156_x_at | CPR8 |
| 201487_at | CTSC |
| 205749_at | CYP1A1 |
| 207913_at | CYP2F1 |
| 206153_at | CYP4F11 |
| 206514_s_at | CYP4F3 |
| 216351_x_at | DAZ4 |
| 203799_at | DCL-1 |
| 212665_at | DKFZP434J214 |
| 201430_s_at | DPYSL3 |
| 211048_s_at | ERP70 |
| 219118_at | FKBP11 |
| 214119_s_at | FKBP1A |
| 208918_s_at | FLJ13052 |
| 217487_x_at | FOLH1 |
| 200748_s_at | FTH1 |
| 201723_s_at | GALNT1 |
| 218885_s_at | GALNT12 |
| 203397_s_at | GALNT3 |
| 218313_s_at | GALNT7 |
| 203925_at | GCLM |
| 219508_at | GCNT3 |
| 202722_s_at | GFPT1 |
| 204875_s_at | GMDS |
| 205042_at | GNE |
| 208612_at | GRP58 |
| 214040_s_at | GSN |
| 214307_at | HGD |
| 209806_at | HIST1H2BK |
| 202579_x_at | HMGN4 |
| 207180_s_at | HTATIP2 |
| 206342_x_at | IDS |
| 203126_at | IMPA2 |
| 210927_x_at | JIB |
| 203163_at | KATNB1 |
| 204017_at | KDELR3 |
| 213174_at | KIAA0227 |
| 212806_at | KIAA0367 |
| 210616_s_at | KIAA0905 |
| 221841_s_at | KLF4 |
| 203041_s_at | LAMP2 |
| 213455_at | LOC92689 |
| 218684_at | LRRC5 |
| 204059_s_at | ME1 |
| 207430_s_at | MSMB |
| 210472_at | MT1G |
| 213432_at | MUC5B |
| 211498_s_at | NKX3-1 |
| 201467_s_at | NQO1 |
| 206303_s_at | NUDT4 |
| 213498_at | OASIS |
| 200656_s_at | P4HB |
| 213441_x_at | PDEF |
| 207469_s_at | PIR |
| 207222_at | PLA2G10 |
| 209697_at | PPP3CC |
| 201923_at | PRDX4 |
| 200863_s_at | RAB1 lA |
| 208734_x_at | RAB2 |
| 203911_at | RAP1GA1 |
| 218723_s_at | RGC32 |
| 200087_s_at | RNP24 |
| 200872_at | S100A10 |
| 205979_at | SCGB2A1 |
| 202481_at | SDR1 |
| 217977_at | SEPX1 |
| 221041_s_at | SLC17A5 |
| 203306_s_at | SLC35A1 |
| 207528_s_at | SLC7A11 |
| 202287_s_at | TACSTD2 |
| 210978_s_at | TAGLN2 |
| 205513_at | TCN1 |
| 201666_at | TIMP1 |
| 208699_x_at | TKT |
| 217979_at | TM4SF13 |
| 203824_at | TM4SF3 |
| 200929_at | TMP21 |
| 221253_s_at | TXNDC5 |
| 217825_s_at | UBE2J1 |
| 215125_s_at | UGT1A10 |
| 210064_s_at | UPK1B |
| 202437_s_at | CYP1B1 |

TABLE 13
Examples of biomarkers

| AFFYID | Gene Name (HUGO ID) |
|---|---|
| 213693_s_at | MUC1 |
| 211695_x_at | MUC1 |
| 207847_s_at | MUC1 |
| 208405_s_at | CD164 |
| 220196_at | MUC16 |
| 217109_at | MUC4 |
| 217110_s_at | MUC4 |
| 204895_x_at | MUC4 |
| 214385_s_at | MUC5AC |
| 1494_f_at | CYP2A6 |
| 210272_at | CYP2B7P1 |
| 206754_s_at | CYP2B7P1 |
| 210096_at | CYP4B1 |
| 208928_at | POR |
| 207913_at | CYP2F1 |
| 220636_at | DNAI2 |
| 201999_s_at | DYNLT1 |
| 205186_at | DNALI1 |
| 220125_at | DNAI1 |
| 210345_s_at | DNAH9 |
| 214222_at | DNAH7 |
| 211684_s_at | DYNC1I2 |
| 211928_at | DYNC1H1 |
| 200703_at | DYNLL1 |
| 217918_at | DYNLRB1 |
| 217917_s_at | DYNLRB1 |
| 209009_at | ESD |
| 204418_x_at | GSTM2 |
| 215333_x_at | GSTM1 |
| 217751_at | GSTK1 |
| 203924_at | GSTA1 |
| 201106_at | GPX4 |
| 200736_s_at | GPX1 |
| 204168_at | MGST2 |
| 200824_at | GSTP1 |
| 211630_s_at | GSS |
| 201470_at | GSTO1 |
| 201650_at | KRT19 |
| 209016_s_at | KRT7 |
| 209008_x_at | KRT8 |
| 201596_x_at | KRT18 |
| 210633_x_at | KRT10 |
| 207023_x_at | KRT10 |
| 212236_x_at | KRT17 |
| 201820_at | KRT5 |
| 204734_at | KRT15 |
| 203151_at | MAP1A |
| 200713_s_at | MAPRE1 |
| 204398_s_at | EML2 |
| 40016_g_at | MAST4 |
| 208634_s_at | MACF1 |
| 205623_at | ALDH3A1 |
| 212224_at | ALDH1Al |
| 205640_at | ALDH3B1 |
| 211004_s_at | ALDH3B1 |
| 202054_s_at | ALDH3A2 |
| 205208_at | ALDH1L1 |
| 201612_at | ALDH9A1 |
| 201425_at | ALDH2 |
| 201090_x_at | K-ALPHA-1 |
| 202154_x_at | TUBB3 |
| 202477_s_at | TUBGCP2 |
| 203667_at | TBCA |
| 204141_at | TUBB2A |
| 207490_at | TUBA4 |
| 208977_x_at | TUBB2C |
| 209118_s_at | TUBA3 |
| 209251_x_at | TUBA6 |
| 211058_x_at | K-ALPHA-1 |
| 211072_x_at | K-ALPHA-1 |
| 211714_x_at | TUBB |
| 211750_x_at | TUBA6 |
| 212242_at | TUBA1 |
| 212320_at | TUBB |
| 212639_x_at | K-ALPHA-1 |
| 213266_at | 76P |
| 213476_x_at | TUBB3 |
| 213646_x_at | K-ALPHA-1 |
| 213726_x_at | TUBB2C |

TABLE 14
shows sample distribution.

| | Training set | | Test set |
|---|---|---|---|
| Representative histopathology types | # samples | # patients | # patients |
| Usual Interstitial pneumonia (UIP) | 136 | 34 | 11 |
| Difficult UIP | 40 | 11 | 7 |
| Favor UIP | 22 | 5 | 4 |
| UIP (lower lobe) + Nonspecific interstitial pneumonia (NSIP) | 5 | 1 | |
| (upper lobe) | | | |
| Difficult UIP (lower lobe) + NSIP (upper lobe) | 4 | 1 | |
| UIP (lower lobe) + Pulmonary hypertension (upper lobe) | 5 | 1 | |
| Favor HP (lower lobe) + Difficult UIP (upper lobe) | | | 1 |
| UIP Total | 212 (60%) | 53 (59%) | 23 (47%) |
| Respiratory bronchiolitis (RB); Smoking-related interstitial | 26 | 7 | 7 |
| fibrosis | | | |
| Hypersensitivity pneumonitis; Favor HP | 19 | 4 | 4 |
| Sarcoidosis | 17 | 5 | 4 |
| NSIP; Cellular NSIP; Favor NSIP | 18 | 5 | 3 |
| Diffuse alveolar damage; DAD with hemosiderosis | 2 | 1 | 2 |
| Amyloid or light chain deposition | | | 1 |
| Bronchiolitis | 12 | 3 | 1 |
| Eosinophilic pneumonia (EP) | 5 | 1 | 1 |
| Exogenous lipid pneumonia | | | 1 |
| Organizing alveolar hemorrhage | | | 1 |
| Organizing pneumonia (OP) | 29 | 7 | |
| Pneumocystis pneumonia (PP) | 4 | 1 | |
| Emphysema | 10 | 3 | |
| Non-UIP Total | 142 (40%) | 37 (41%) | 26 (53%) |
| Total | 354 | 90 | 49 |

TABLE 15

| | | | | # genes |
|---|---|---|---|---|
| | | | Total | overlapping |
| | | | number of | with those |
| | Up- | Down- | differentially | from all |
| | regulated | regulated | expressed | non-UIP |
| | genes | genes | genes | samples |
| All non-UIP (N = 147) | 55 | 96 | 151 | 151 (100%) |
| Bronchiolitis (N = 10) | 41 | 34 | 75 | 6 (8%) |
| HP (N = 13) | 32 | 53 | 85 | 14 (16%) |
| NSIP (N = 12) | 37 | 49 | 86 | 13 (15%) |
| OP (N = 23) | 1 | 15 | 16 | 31 (52%) |
| RB (N = 16) | 549 | 152 | 701 | 64 (9%) |
| Sarcoidosis (N = 11) | 448 | 726 | 1174 | 93 (8%) |

Table 15 shows the number of significantly expressed genes (p-adjusted<0.05, fold change>2) between each non-UIP subtype and UIP samples (n=212). The number of differentially expressed genes overlapping with those between UIP and non-UIP samples is summarized in the third column.

TABLE 16

| Classifier | Between-run | Intra-run (Residual) | Inter-run (Total) |
|---|---|---|---|
| Ensemble | 0.28 (4.0%) | 0.37 (5.3%) | 0.46 (6.5%) |
| Penalized | 0.10 (2.6%) | 0.19 (4.9%) | 0.22 (5.6%) |
| logistic regression | | | |

Table 16 shows an estimation of variability of scores from the two classifiers using linear mixed effect models. The percentage (%) may be the ratio of estimated variability to the range between %5 and 95% quantiles in classification scores.

Classifier described herein may diagnosis a condition, such as IPF or lung cancer, while avoiding an invasive procedure. One disadvantage of an unsupervised clustering analysis may be an inability to (a) distinguish a malignant tissue from a benign tissue, (b) distinguish a UIP pattern from a non-UIP pattern, (c) distinguish a sample having a particular expression pattern from another sample that may not have the particular expression pattern or (d) any combination thereof because of (i) a small sample size, (ii) disease heterogeneity (for example heterogeneity in a non-UIP pattern disease subtype), (iii) pooling and batch effects of different samples, or (iv) any combination thereof. A trained machine learning algorithm may overcome these disadvantages. Methods described herein may eliminate the need for an invasive procedure and provide a non-invasive prognostic tool, diagnostic tool, or a combination thereof with high clinical accuracy despite the limitation of a small sample size, disease heterogeneity, or pooling and batch effects of different samples. In some cases, RNA-seq data may be input into the machine learning algorithm. Heterogeneity may occur within samples obtained from the same subject. For example, histopathology features may not be uniform across a tissue (such as a lung tissue) and gene expression profiles may vary depending on a location from which a sample is obtained. Heterogeneity may occur within a disease. For example, a presence of a non-UIP pattern may comprise more than one disease subtype such as a collection of heterogeneous diseases.

In some cases, 1, 2, 3, 4, 5, 6, 7, 8, 9, 10 or more samples may be collected from a subject and separately analyzed. In some cases, 2 samples may be collected from a subject and separately analyzed. In some cases, 3 samples may be collected from a subject and separately analyzed. In some cases, 4 samples may be collected from a subject and separately analyzed. In some cases, 5 samples may be collected from a subject and separately analyzed. In some cases, 6 samples may be collected from a subject and separately analyzed. In some cases, 7 samples may be collected from a subject and separately analyzed. In some cases, 8 samples may be collected from a subject and separately analyzed. In some cases, 9 samples may be collected from a subject and separately analyzed. In some cases, 10 samples may be collected from a subject and separately analyzed. In some cases, from 1 to 10 samples may be collected form a subject and separately analyzed. In some cases, from 1 to 5 samples may be collected form a subject and separately analyzed. In some cases, from 1 to 20 samples may be collected form a subject and separately analyzed.

A classifier, such as a locked classifier, may yield a substantially similar accuracy, NPV, PPV, sensitivity, specificity, or any combination thereof in an independent test set as compared to a validation set (that may be used to validate the classifier). A classifier may maintain a substantially similar accuracy, NPV, PPV, sensitivity, specificity, or any combination thereof over at least about 5 independent test samples. A classifier may maintain a substantially similar accuracy, NPV, PPV, sensitivity, specificity, or any combination thereof over at least about 10 independent test samples. A classifier may maintain a substantially similar accuracy, NPV, PPV, sensitivity, specificity, or any combination thereof over at least about 50 independent test samples. A classifier may maintain a substantially similar accuracy, NPV, PPV, sensitivity, specificity, or any combination thereof over at least about 100 independent test samples. A classifier may maintain a substantially similar accuracy, NPV, PPV, sensitivity, specificity, or any combination thereof over at least about 500 independent test samples. A classifier may maintain a substantially similar accuracy, NPV, PPV, sensitivity, specificity, or any combination thereof over at least about 1000 independent test samples. A classifier may maintain a substantially similar accuracy, NPV, PPV, sensitivity, specificity, or any combination thereof over from about 1 to about 10 independent test samples. A classifier may maintain a substantially similar accuracy, NPV, PPV, sensitivity, specificity, or any combination thereof over from about 1 to about 100 independent test samples. A classifier may maintain a substantially similar accuracy, NPV, PPV, sensitivity, specificity, or any combination thereof over from about 1 to about 500 independent test samples. A classifier may maintain a substantially similar accuracy, NPV, PPV, sensitivity, specificity, or any combination thereof over from about 1 to about 1000 independent test samples. A classifier may maintain a substantially similar accuracy, NPV, PPV, sensitivity, specificity, or any combination thereof over from about 1 to about 5000 independent test samples. Independent test samples may be obtained from a subject.

To maintain substantially similar accuracy, NPV, PPV, sensitivity, specificity, or any combination thereof over a plurality of independent test samples, batch effects may be removed. Removal of biomarkers yielding high variability across samples may be removed from selection features of a classifier or from downstream analysis. Biomarkers highly sensitive to batch effects may be removed from downstream analysis or removed from feature selection. A classifier may not substantially vary performance (such as accuracy, NPV, PPV, sensitivity, or specificity) over a plurality of independent sample runs.

The methods may include identifying subjects having heterogeneity within a plurality of samples obtained from a subject. For example, the methods may include identifying a subject having a sample assigned a non-UIP pattern and another sample from the same subject assigned a UIP-pattern. Heterogeneity in samples from the same subject may be observed in histopathologic diagnosis, gene expression, or a combination thereof. For example, UIP and non-UIP pattern diseases may be heterogeneous. Biomarkers that may distinguish or diagnose a non-UIP pattern disease may not be applicable to distinguishing or diagnosing another non-UIP pattern disease. A new set of biomarkers may be developed for each disease, disease sub-type, UIP pattern, or non-UIP pattern disease. Biomarkers that may distinguish or diagnose a presence of a non-UIP pattern disease may be applicable to distinguishing or diagnosis another non-UIP pattern disease.

Samples in the training set may comprise a plurality of conditions (such as diseases or disease subtypes). Samples in an independent test set may comprise a plurality of conditions (such as disease or disease subtypes). Samples in an independent test set may comprise a least one disease or disease subtype that is different from the samples in the training set. Samples in the training set may comprise a least one disease or disease subtype that is different from the samples in the independent test set. Samples in the independent test set may comprise at least two additional diseases or disease subtypes than the samples in the training set. For example, the at least two additional diseases or disease subtypes may be amyloid or light chain deposition, exogenous lipid pneumonia, and organizing alveolar hemorrhage, or any combination thereof. One or more new diseases or disease subtypes may emerge from an independent test set that may not be included in a training set. Samples in the training set may comprise at least two additional diseases or disease subtype than the samples in the independent test set.

The methods may include evaluating classifier performance with in silico samples. In silico samples may simulate mixing of in vitro samples in an independent test set, particularly when a sample size may be small. In silico samples may also aid in determining decision boundaries of a classifier, optimal number of samples required to achieve optimal classifier performance, or a combination thereof. The methods may be applicable to pooled samples, for example, when a small sample size may be present.

A small sample size may be samples obtained from less than 100, 90, 80, 70, 60, 50, 40, 30, 25, 20, 15, 10, or 5 different subjects. A small sample size may be a plurality of samples obtained from about 50 to about 100 different subjects. A small sample size may be a plurality of samples obtained from about 1 to about 50 different subjects. A small sample size may be a plurality of samples obtained from about 1 to about 100 different subjects. A small sample size may be a plurality of samples obtained from about 1 to about 200 different subjects. A small sample size may be a plurality of samples obtained from about 1 to about 10 different subjects. A small sample size may be a plurality of samples obtained from about 1 to about 5 different subjects. A small sample size may be a plurality of samples obtained from about 1 to about 2 different subjects. A small sample size may be a plurality of samples obtained from about 1 to about 15 different subjects. A small sample size may be a plurality of samples obtained from about 1 to about 8 different subjects. A small sample size may be a plurality of samples obtained from about 5 to about 50 different subjects. A small sample size may be a plurality of samples obtained from about 5 to about 100 different subjects. A small sample size may comprise a small sample size of independent test samples or training samples. A small sample size may be indicative of a limited access to subjects—such as subjects having a rare subtype of a disease. A small sample size may be expanded by including replicates of a single sample, such as 1, 2, 3, 4, 5, or more replicates of a single sample. A small sample size may be expanded by including from about 1 to about 2 replicates of a single sample. A small sample size may be expanded by including from about 1 to about 3 replicates of a single sample. A small sample size may be expanded by including from about 1 to about 4 replicates of a single sample. A small sample size may be expanded by including from about 1 to about 5 replicates of a single sample. A small sample size may be expanded by including from about 1 to about 10 replicates of a single sample. A small sample size may be expanded by including from about 1 to about 15 replicates of a single sample. A small sample size may be expanded by including from about 1 to about 20 replicates of a single sample.

### EXAMPLES

### Example 1

Background—To accurately diagnose Idiopathic Pulmonary Fibrosis (IPF) while avoiding invasive procedures, a classifier may be developed using RNA-seq data that identifies histopathologic pattern of usual interstitial pneumonia (UIP), a hallmark characteristic of IPF. This approach may challenge encountered in the development of a classifier, including sample size, heterogeneity, and batch effects, while applying machine learning to genomic data in clinical settings.

Methods—Exome-enriched RNA sequencing may be performed on 354 individual transbronchial biopsies (TBBs) from 90 patients to use in the training algorithms. Pooled TBB samples composed of 3-5 individual TBBs from 49 additional patients as an independent validation may be sequenced. Unsupervised clustering and differentially expressed gene analysis may be performed to characterize disease heterogeneity and to select genomic features that may distinguish between UIP from non-UIP. To overcome the small sample size and potential disease heterogeneity, machine learning algorithms may be trained using multiple samples per patient. Simulated in silico mixed samples to mimic pooled samples of the test set may be evaluated. The machine learning algorithm may be validated on the test set, and its robustness may be further evaluated using technical replicates across multiple batches.

Results—Unsupervised clustering and differential gene expression analyses may show high heterogeneity within patients, particularly among the non-UIP group. The developed classifiers, using penalized logistic regression model and ensemble models may classify histopathologic UIP with a receiver-operator characteristic area under the curve (AUC) of about 0.9 in cross-validation, when multiple samples may be tested per patient. A decision boundary may be defined to optimize specificity at ≥85% using TBB pools that may be simulated in silico from the individual training set samples. The penalized logistic regression model may show greater reproducibility across technical replicates, and may be chosen as the final model. The final model may show sensitivity of 70% and specificity of 88% in the independent test set, using samples that may be pooled in the laboratory prior to molecular testing.

Conclusions—Overcoming challenges of sample size, disease and sampling heterogeneity, pooling and batch effects, a method as described here may provide a highly accurate and robust classifier for the identification of UIP, leveraging machine learning and RNA-seq.

Introduction—Interstitial lung disease (ILD) consists of a variety of diseases affecting the pulmonary interstitium with similar clinical presentation; idiopathic pulmonary fibrosis (IPF) may be the most common ILD with the worst prognosis. The cause of IPF remains largely unknown making accurate and timely diagnoses challenging. An accurate diagnosis for IPF often entails multidisciplinary evaluation of clinical, radiologic and histopathologic features [Flaherty et al, 2004 and Travis et al, 2013, which are entirely incorporated herein by reference], and patients frequently suffer an uncertain and lengthy process. In particular, determining the presence of usual interstitial pneumonia (UIP), a hallmark characteristic of IPF, often requires histopathology via invasive surgery that may not be an option for sick or elderly patients. Furthermore, the quality of the histopathology reading may be highly variable across clinics [Flaherty et al, 2007, which is entirely incorporated herein by reference]. Thus, a consistent, accurate, non-invasive diagnosis tool to distinguish UIP from non-UIP without the need for surgery may be critical to reduce the suffering of patients and to enable physicians to reach confident clinical diagnoses faster and make better treatment decisions.

To build this new diagnostic tool, exome-enriched RNA sequencing data may be utilized from transbronchial biopsy samples (TBBs) collected via bronchoscopy, a less invasive procedure compared to surgery. Several studies have revealed that genomic information in transcriptomic data may be indicative of phenotypic variation such as cancer and other chronic disease [Tuch et al 2010, Twine et al 2011, which are entirely incorporated herein by reference]; and that complex traits may be driven by large number of genes spread across the whole genome including ones with no apparent relevance to disease [Boyle et al, 2017, which is entirely incorporated herein by reference]. More importantly, the feasibility of identifying UIP using transcriptomic data has been established [Pankratz et al, 2017, which is entirely incorporated herein by reference]. The methods and systems as described herein provide analytical solutions to such problems.

Machine learning methods have been extensively applied to solve biomedical problems, and have deepened our understanding of diseases such as breast cancer [Sorlie et al., which is entirely incorporated herein by reference], and glioblastoma [Brennan et al., which is entirely incorporated herein by reference], by allowing researchers to construct biological pathways, identify clinically relevant diseases and better predict disease risk. However, recent advances in machine learning may be often designed for large data sets such as medical imaging data and social media data. Yet, clinical studies, including this one, often have limited sample sizes due to the challenges in accruing patients. The issue may be more pronounced in the present example since many patients may be too sick to allow biopsy samples; among the ones collected, a substantial proportion yielded non-diagnostic results, rendering them unsuitable for supervised learning. In addition, the non-UIP category may not be one disease, but a collection of heterogeneous diseases. This, coupled with the small sample size, may indicate that small numbers of samples may be available in each non-UIP disease category, making the classification even more challenging. Another unique feature of this example may be heterogeneity within a patient. Histopathology features may not be uniform across the entire lung and genomic signatures vary depending on the location of the biopsy sample [Kim et al, which is entirely incorporated herein by reference]. To better understand such heterogeneity, multiple samples (up to 5) per patient may be collected and sequenced separately for patients in the training set. This data set may represent both a challenge and an opportunity, which may be described in details in later sections.

Because a classifier may serve as the foundation for a diagnostic product, there may be two additional requirements. First, for cost-effectiveness, only one sequencing run per patient may be commercially viable and the independent test set may need to reflect this reality. Analytically bridging individual samples in the training set and pooled samples in the test set may become a necessity. Secondly, it may be important that a final locked classifier not only performs well on the independent test set, but may also maintain performance for all incoming future samples. Therefore, developing a classifier that may be highly robust to foreseeable batch effects in the future may become critically important.

In the following sections, some of the challenges with quantitative analysis may be illustrated, practical solutions to overcome those challenges may be described, evidence of improvement may be shown, and limitations of these approaches may be discussed.

Materials and Methods

Study Design

Patients under medical evaluation for ILD that may be 18 years of age or older and may be undergoing a planned, clinically indicated lung biopsy procedure to obtain a histopathology diagnosis may be eligible for enrollment in a multi-center sample collection study (BRonchial sAmple collection for a noVel gEnomic test; BRAVE) [Pankratz et al]. Patients for whom a bronchoscopy procedure may not be indicated, not recommended or difficult may not be eligible for participation in the study. Patients may be groups based on the type of biopsy being performed for pathology: BRAVE-1 patients may undergo surgical lung biopsy (SLB), BRAVE-2 patients may undergo TBB for pathology, and BRAVE-3 patients may undergo cryobiopsy. The study may be approved by institutional review boards at each institution and all patients may be provided informed consent prior to their participation.

During study accrual, 201 BRAVE patients may be prospectively divided into a group of 113 considered for use in training (enrolled December 2012 to July 2015) and 88 may be used in validation (enrolled August 2014 and May 2016). The training group may ultimately yield 90 patients with usable RNA sequence data and reference standard pathology truth labels that may be used to train and cross-validate the models. The validation group may yield 49 patients that met prospective test set inclusion criteria related to sample handling, sample adequacy, and the determination of reference standard truth labels. All clinical information related to the test set, to include reference labels and associated pathology may be blinded to the algorithm development team until after the classifier parameters may be finalized, locked, and the test set may be prospectively scored.

Total RNA may be extracted and input into TruSeq RNA Access Library Prep procedure (Illumina, San Diego, Calif.) to enrich for expressed exonic sequences, and sequenced on the NextSeq 500 instruments with a NextSeq v2 chemistry 150 cycle kit (Illumina, San Diego, Calif.). For the training set, RNA sequencing data may be generated separately for each of 354 individual TBB samples from 90 patients and eight additional TBB samples may be chosen for quality control and sequenced repeatedly over eight different batches, which may be referred to as sentinels. For the independent test set, total RNA extracted from available TBB samples for each patient may be mixed by equal mass and sequenced using the same procedure as that for the training set but at a later time on a different batch. Therefore, for the training set, there may be up to 5 sequencing data per patient, one corresponding to an individual TBB sample; in contrast, for the test set, there may be 1 sequencing data per patient, since all TBB samples and the corresponding RNA material derived from the same test patient may be pooled together prior to sequencing which may be representative of how a commercial samples may be run.

Pathology Reviews and Label Assignment

Histopathology diagnoses may be determined centrally by a consensus of three expert pathologists using biopsies and slides collected specifically for pathology, following processes described [Pankratz et al and Kim et al]. The central pathology diagnoses may be determined separately for each lung lobe samples for pathology. A reference standard label may then be determined for each patient from the lobe-level diagnoses according to the following rules. If any lob may be diagnosed as any UIP subtype, e.g., classic UIP (all features of UIP may be present), difficult UIP (less than all features of classic UIP may be well represented), favor UIP (fibrosing interstitial process with UIP leading the differential), or any combination of these, then ‘UIP’ may be assigned as the reference label for that patient. If any lung lobe may be diagnosed with a ‘non-UIP’ pathology condition [Pankratz et al] and any other lobe may be non-diagnostic or may be diagnosed with unclassifiable fibrosis, then ‘non-UIP’ may be assigned as the patient level reference label. When all lobes may be diagnostic for unclassifiable fibrosis (e.g., chronic interstitial fibrosis, not otherwise classified or ‘CIF, NOC’) or may be non-diagnostic, then no reference label may be assigned and the patient may be excluded. This patient-level reference label process may be identical between training and testing sets, however individual TBB samples in the training set may be directly inherited sample level reference labels from the lung lobe of origin, in addition to the reference label determined at the patient level.

Molecular Testing, Sequencing Pipeline, and Data QC

Up to five TBB samples may be sampled from each patient by bronchoscopy. Typically, two upper lobe and three lower lobe samples may be collected during the clinically indicated diagnostic procedure. TBB samples for molecular testing may be placed into a nucleic acid preservative and may be stored at 4° C. for up to 18 days, prior to and during shipment to the development laboratory, followed by frozen storage. Total RNA may be extracted, may be quantitated, may be pooled by patient where appropriate, and 15 ng input into the TruSeq RNA Access Library Prep procedure (Illumina, San Diego, Calif.), which may enrich for the coding transcriptome using multiple rounds of amplification and hybridization to probes specific to exonic sequences. Libraries which met in-process yield criteria may be sequenced on NextSeq 500 instruments (2×75 bp paired-end reads) using the High Output kit (Illumina, San Diego, Calif.). Raw sequencing (FASTQ) files may be aligned to the Human Reference assembly 37 (Genome Reference Consortium) using the STAR RNA-seq aligner software [Dobin et al, which is entirely incorporated herein by reference]. Raw read counts for 63,677 Ensembl annotated gene-level features may be summarized using HTSeq [Anders et al, 2015, which is entirely incorporated herein by reference]. Data quality metrics may be generated using RNA-SeQC [DeLuca et al, which is entirely incorporated herein by reference]. Library sequence data which met minimum criteria for total reads, mapped unique reads, mean per-base coverage, base duplication rate, the percentage of bases aligned to coding regions, the base mismatch rate, and uniformity of coverage within genes may be accepted for use in downstream analysis.

Normalization

Sequence data may be filtered to exclude any features that may not be targeted for enrichment by the library assay, resulting in 26,268 genes. For the training set, expression count data for 26,268 Ensembl genes may be normalized by sizefactor estimated with the median-of-ratio method and transformed to approximately log 2 by variance-stabilizing transformation (VST) using a parametric method, which may be a closed-form expression (DESeq2 package) [Love et al, 2014, which is entirely incorporated herein by reference]. The vector of geometric approaches and VST from the training set may be frozen and separately reapplied to the independent test set for the normalization to mimic future clinical patterns.

For algorithm training and development, RNA sequence data may be generated separately for each of 354 individual TBB samples from 90 patients. Eight additional TBB samples (‘sentinels’) may be replicated in each of eight processing runs, from total RNA through to sequence data, to monitor for batch effects. For validation, total RNA may be extracted from a minimum of three and a maximum of five TBBs per patient may be mixed by equal mass within each patient prior to library preparation and sequencing. Patients in the training set thus may contribute up to five sequence libraries to training, whereas patients in the test set may be represented by a single sequenced library, analogous to the planned testing of clinical samples.

Differential Expression Analysis

Whether differentially expressed genes found using a standard pipeline [Anders et al., 2013, which is entirely incorporated herein by reference], may be used directly to classify UIP from non-UIP samples may be explored. Differentially expressed genes may be identified using DESeq2, a Bioconductor R package [Love et al. 2014]. Raw gene-level expression counts of the training set may be used to perform the differential analysis. A cutoff of p-value<0.05 after multiple-testing adjustment and fold change>2 may be used to select differentially expressed genes. Within the training set, pairwise differential analyses may be performed between all non-UIP and UIP samples, and between UIP samples and each non-UIP disease with more than 10 samples available, including bronchiolitis (N=10), hypersensitivity pneumonitis (HP) (N=13), nonspecific interstitial pneumonia (NSIP) (N=12), organizing pneumonia (OP) (N=23), respiratory bronchiolitis (RB) (N=16), and sarcoidosis (N=11). Principal component analysis (PCA) plots of all the training samples may be generated using differentially expressed genes identified above.

Gene Expression Correlation Heatmap

The correlations r² values of samples in 6 representative patients may be computed using their VST gene expression, and a heatmap of the correlation matrix with patient order preserved may be plotted to visualize intra- and inter-patient heterogeneity in gene expression. The 6 patients may be selected to represent the full spectrum of with-in patient heterogeneity including two non-UIP and two UIP patients with the same or similar labels between upper and lower lobes, as well as one UIP and one non-UIP patients each having different labels at upper versus lower lobes. The heatmap may be generated using the heatmap.2 function of the gplots R package.

Classifier Development

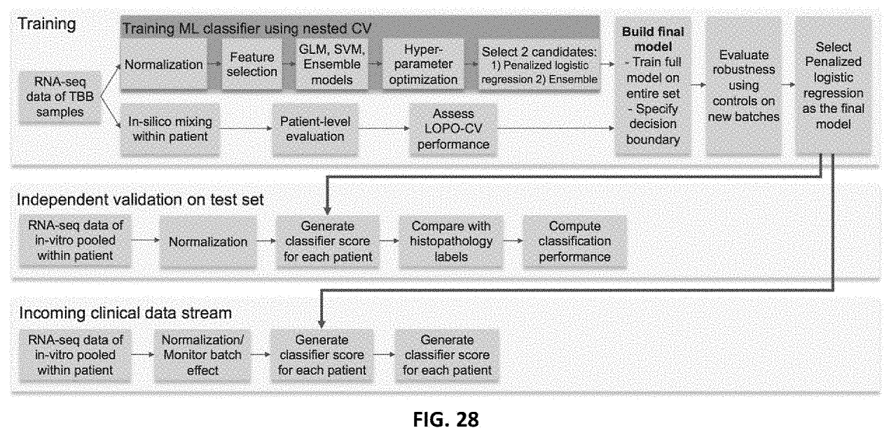

The development and evaluation of a classifier may be summarized in FIG. 28. A goal may be to build a robust binary classifier may be built on TBB samples to provide accurate and reproducible UIP/non-UIP predictions, and to meet the clinical need to reduce invasive procedures for ILD patients. A high specificity test (specificity>85%) may be designed to ensure a high positive predictive value. When the test may predict UIP, that result may be associated with high confidence.

Feature Filtering for Classifier Development

First, features that may not be biologically meaningful or less informative may be removed due to low expression level without variation among samples may be filtered. Genes annotated in Ensembl as pseudogenes, ribosomal RNAs, individual exons in T-cell receptor or Immunoglobulin genes and non-informative and low expressed genes may be excluded with raw counts expression level<5 for the entire training set or expressed with count>0 for less than 5% of samples in the training set.

Genes with highly variable expression in the same sample that maybe processed in multiple batched may be excluded, as this may suggest sensitivity to technical, rather than biological factors. To identify such genes, a linear mixed effect model may be fitted on the sentinel TBB samples processed across multiple assay plates. This model may be fitted for each gene separately where gᵢⱼ may be the gene expression of sample j and batch i, μ may be the average gene expression

gᵢⱼ=μ+βsampleᵢⱼ+batchᵢ+eᵢⱼ (1)

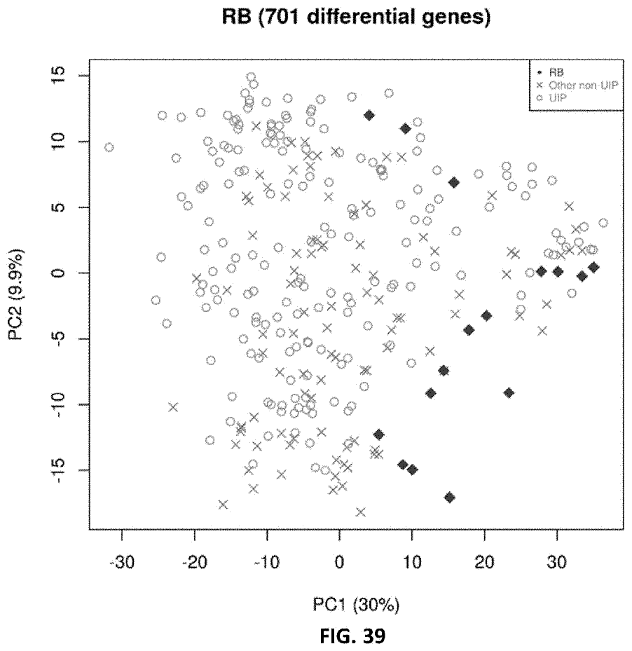

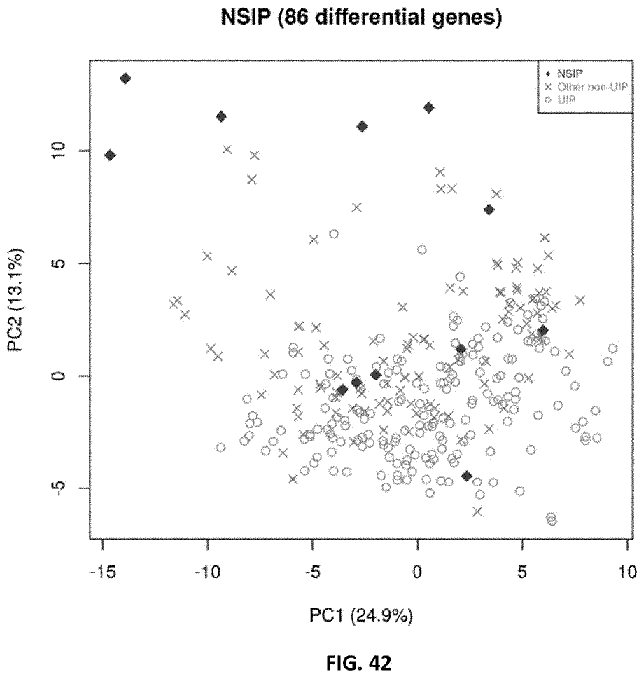

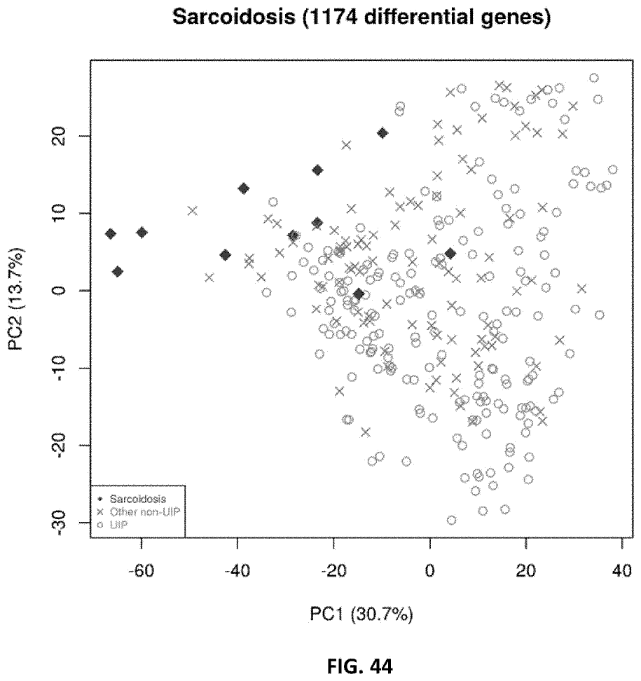

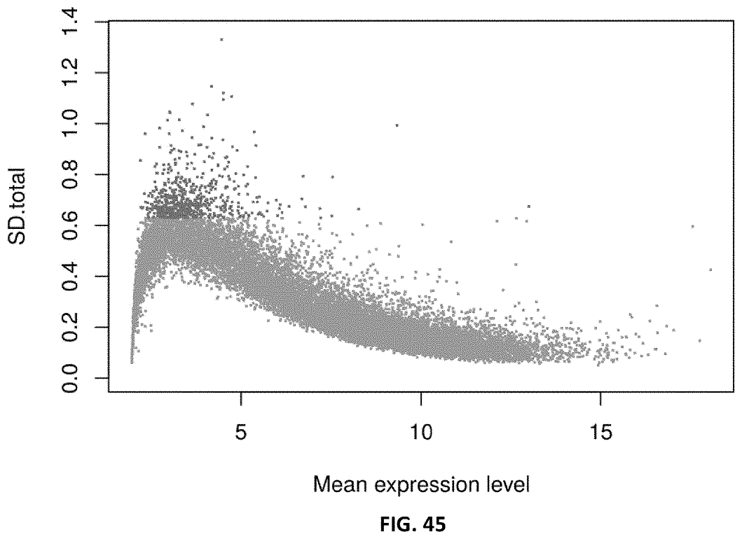

for the entire set, sampleᵢⱼ may be a fixed effect of biologically different samples, and batch, may be the batch-specific random effect. The total variation may be used to identify highly variable genes; the top 5% of genes by this measure may be excluded (FIG. 39-44). As a result, 17,601 Ensembl genes may remain as candidates for the downstream analysis.

In Silico Mixing within Patient

The classifiers may be trained and optimized on individual TBB samples to maximize sampling diversity and the information content available during the feature selection and weighting process. Multiple TBB samples may be pooled at the post-extraction stage, as RNA, and the pooled RNA may be processed in a single reaction through library prep, sequencing and classification [Pankratz et al]. Whether a classifier developed on individual samples may achieve high performance on pooled samples may be evaluated. A method may be developed to simulate pooled samples “in silico” from individual sample data. First, raw read counts may be normalized by sizefactor computed using geometric approaches across genes within the entire training set. The normalized count Cᵢⱼ for sample i=1, . . . , n and gene j=1, . . . , m may be computed by

Cᵢⱼ=Kᵢⱼ/Sⱼ

where

$${s}_{j} = {median}_{i} \frac{{K}_{ij}}{{( {\prod }_{v = 1}^{m} {K}_{iv} )}^{1 \text{/} m}}$$

and Kᵢⱼ may be the raw count for sample i and gene j. Then, for each training patient p=1, . . . , P, in silico mixed count K^{p}ᵢⱼ may be defined by

$${K}_{ij}^{p} = \frac{1}{{n}_{p}} {\sum }_{i ∈ I ( p )} {C}_{ij}$$

where I (p) may be the index set of individual sample i that may belong to patient p. The frozen variance stabilizing transformation (VST) in the training set may applied to K^{p}ᵢⱼ.

Training Classifiers

As the test may be intended to recognize and call a reference label defined by pathology, the reference label may be defined to be the response variable in classifier training [Tuch et al], and the exome-enriched, filtered and normalized RNA sequence data as the predictive features. Multiple classification models may be evaluated, to include random forest, support vector machine (SVM), gradient boosting, neural network and penalized logistic regression [Dobson et al, which is entirely incorporated herein by reference]. Each classifier may be evaluated based on 5-fold cross-validation and leave-one-patient-out cross-validation (LOPO CV) [Friedman et al, which is entirely incorporated herein by reference]. Ensemble models may also be examined by combining individual machine learning methods via weighted average of scores of individual models.

To minimize overfitting, during training and evaluation, each cross-validation fold may be stratified such that all data from a single patient may be either included or held out from a given fold. Hyper-parameter tuning may be performed within each cross-validation split in a nested-cross validation manner [Krstajic D et al, 2014, which is entirely incorporated herein by reference]. A random search and one standard error rule [Hastie, Tibshirani and Friedman, 2009, which is entirely incorporated herein by reference] may be chosen for selection of best parameters from inner CV to further minimize potential overfitting. Ultimately, hyper-parameter tuning may be repeated on the full training set to define the parameters for in the final locked classifier. The pipeline of training various machine learning algorithms may be automated and performed using R packages: DESeq2, hclust, cv.glmnet, caret and caretEnsemble.

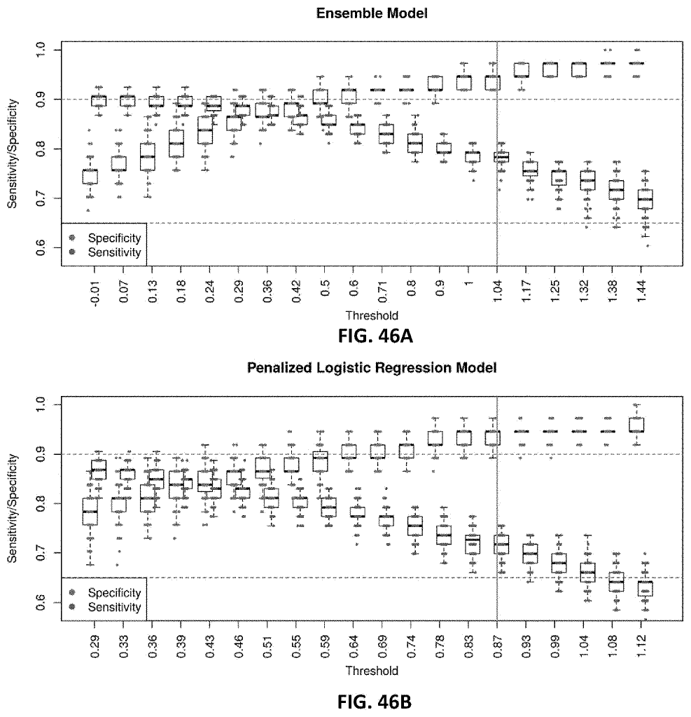

Best practices for a fully independent validation may require that all classifier parameters, including the test decision boundary may be prospectively defined. This therefore may be done using only the training set data. Since the test set may classify pooled TBBs at the patient-level, the proposed in silico mixing model may be used to simulate the distribution of patient-level scores within the training set. Within-patient mixtures may be simulated 100 times at each LOPO CV-fold, with gene-level technical variability added to the VST expressions. The gene-level technical variability may be estimated using the mixed effect model. Equation (1) on the TBB samples may be replicated across multiple processing batches. The final decision boundary may be chosen to optimize specificity (>0.85) without severely compromising sensitivity (≥0.65). Performance may be estimated using patient-level LOPO CV scores from replicated in silico mixing simulation. To be conservative for specificity, a criterion for averaged specificity of greater than 90% to choose a final decision boundary. For decision boundaries with similar estimated performances in simulation, the decision boundary with highest specificity may be chosen, FIG. 46A-B.

Evaluate Batch Effect and Monitoring Scheme for Future Samples

To ensure the extensibility of classification performance to a future, unseen clinical patient population, it may be crucial to ensure there may be no severe technical factor, referred as batch effects that may cause globe shifts, rotations, compressions, or expansions of score distributions over time. To quantify batch effects in existing data and to evaluate the robustness of the candidate classifiers to observable batch effects, the scored nine different TBB samples, triplicated within each batch and processed across three different processing batches, and used linear mixed effect model to evaluate variability of scores for each classifier. The model that may be more robust against batch effect, as indicated by low score variability in linear mixed models, may be chosen as the final model for independent validation. To monitor batch effects, UIP and non-UIP control samples may be processed in each new processing batch. To capture a potential batch effect, scores of these replicated control samples may be compared and whether estimated score variability remains smaller than the pre-specified threshold, σₛᵥ, may be determined in training using the in silico patient-level LOPO CV scores.

Independent Validation

A final candidate classifier may be prospectively validated on a blinded, independent test set of TBB samples from 49 patients. Classification scores on the test set may be derived using the locked algorithm and may be compared against the pre-set decision boundary to give the binary prediction of UIP vs. non-UIP calls: classification score above the decision boundary may be called UIP, equal or below the decision boundary may be called non-UIP. The continuous classification scores may be compared against the histopathology labels to construct the ROC and calculate the AUC. The binary classification predictions may be compared against the histopathology labels to calculate the binary classification performance such as sensitivity and specificity.

Score Variability Simulation

In a clinical setting, it may be important to monitor if classification scores of future clinical samples remain stable and may not be affected by potential technical factors. To do this, the limit of score variability that the classifier can tolerate may need to be addressed prospectively. Under the assumption that the LOPO CV scores can represent the distribution of classification scores in the targeted population, a simulation may be performed for sensitivity, specificity and flip-rate between UIP and non-UIP calls. As a first step, a simulated noise may be added to in silico patient-level LOPO CV scores, where a noise may be simulated as e˜N (0, σ²), and σ² may be 0, 0.01, . . . , 10. Then, sensitivity, specificity and flip-rate may be computed using scores with the simulated noise. The simulation may be replicated 1,000 times. Using 1,000 sets of simulated scores, individual thresholds, σ_{spec}, σₛₑₙₛ and σ_{flip} may be defined as the maximum of standard deviation, a, of a noise where the estimated (averaged) specificity>0.9, sensitivity>0.65, and flip-rate<0.15, respectively. The final threshold for classification score variability may be defined as

σₛᵥ=min(σ_{spec},σₛₑₙₛ,σ_{flip})

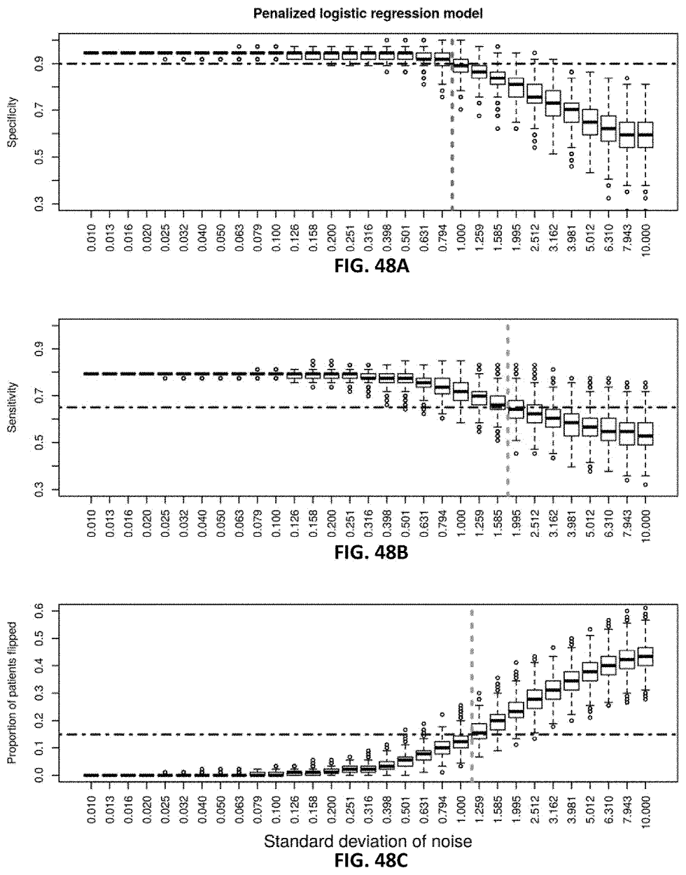

The thresholds for the ensemble model may be 0.9, 1.8, and 1.15 for specificity, sensitivity, and flip-rate, respectively and the final threshold may be σ^{E}ₛᵥ=0.9 (FIG. 48A-C). The thresholds for the penalized regression model may be 0.48, 0.78 and 0.68 for specificity, sensitivity, and flip-rate, respectively and the final threshold may be σ^{PL}ₛᵥ=0.48.

Results

Distribution of ILD Diseases

Table 14 summarizes a distribution of patients for ILD diseases within UIP and non-UIP groups. Among collected patients, the prevalence of patients with UIP pattern may be higher in the training set (59%) than in the test set (47%) with p-value of 0.27. Three patients in the training set and one patient in the test set may have potential heterogeneity within patient: one lobe may be assigned as one of several non-UIP diseases (nonspecific interstitial pneumonia, pulmonary hypertension, or favor hypersensitivity pneumonitis), while the other lobe may be assigned a UIP pattern, driving the final patient-level label as UIP.

The non-UIP group may include a diversity of heterogeneous diseases that may be commonly encountered in clinical practice. Due to the small sample size, several diseases may have one or two patients. Three new diseases—amyloid or light chain deposition, exogenous lipid pneumonia, and organizing alveolar hemorrhage—may be present in the test set, which may not exist in the training set.

Intra-Patient Heterogeneity

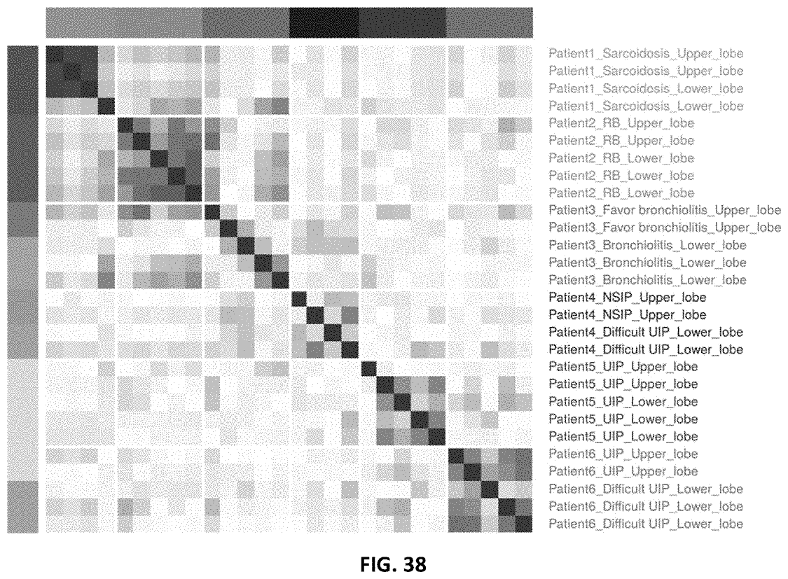

Heterogeneity in samples from the same patient may be observed in both histopathologic diagnosis and gene expression. Three such patients with diseases across UIP and non-UIP groups, may pose a computational challenge for patient-level diagnostic classification. The correlation matrix of samples from six patients may also reveal prominent intra- and inter-patient variability in expression profiles (FIG. 38). FIG. 38 shows two non-UIP patients with the same labels across different lobes and similar gene expression pattern (patients 1 and 2 in FIG. 38), two UIP patients with the same or similar labels and highly correlated expression profiles (patients 5 and 6 in FIG. 38), as well as one UIP and one non-UIP patient with dissimilar labels and heterogeneous expression (patients 3 and 4 in FIG. 38), providing a representative visualization of the full spectrum of heterogeneity that may be observed within and across patients.

DE Analysis Between UIP and Non-UIP

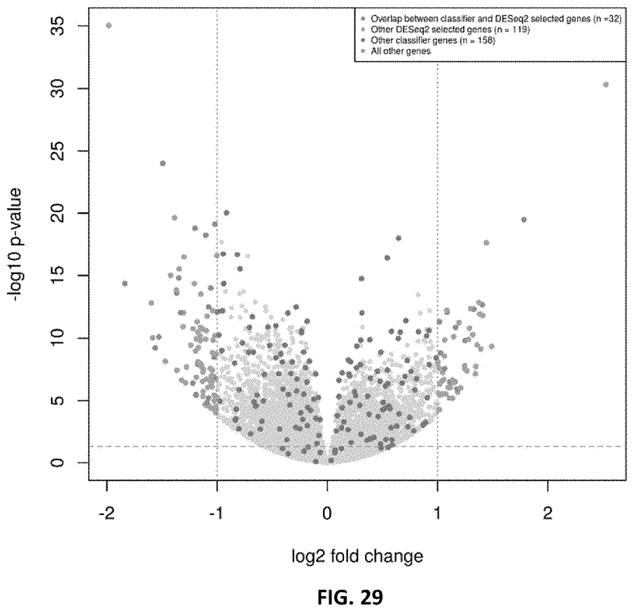

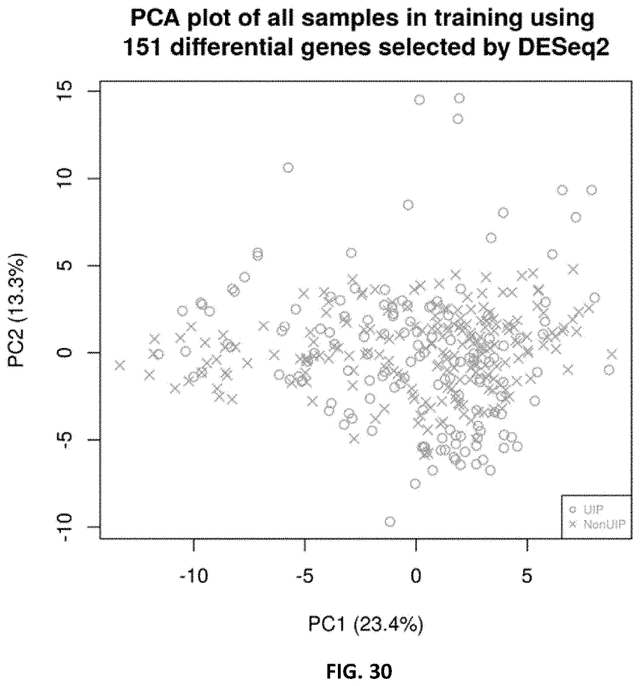

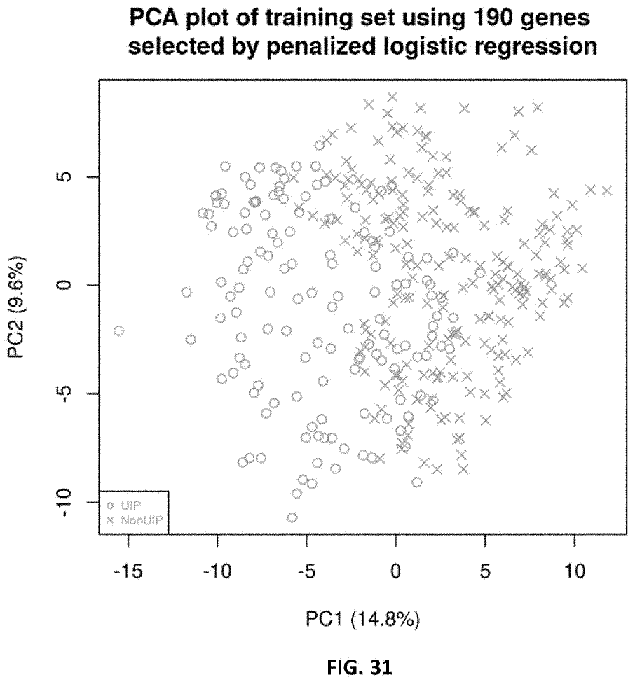

It may first be investigated whether differentially expressed genes found by DESeq2 between UIP and non-UIP may be predictive of the two diagnostic classes. 151 significantly differentially expressed genes may be identified between UIP and non-UIP (adjusted p<0.05, fold change>2), with 55 up-regulated and 96 down-regulated genes in UIP (FIG. 29, Table 15). However, using these differentially expressed genes alone it may be challenging to separate the two classes perfectly, as shown by the PCA plot (FIG. 30). In contrast, PCA spanned by the 190 classifier genes may separate the two classes much better (FIG. 31).

Heterogeneity in Patients of Non-UIP Diseases

Heterogeneity may be observed in gene expression of non-UIP samples, consisting of more than a dozen clinically defined diseases. Genes may be identified that may be significantly different (adjusted p<0.05, fold change>2) between UIP samples and each non-UIP disease subtype with a sample size greater than 10 (Table 15). The higher the number of differentially expressed genes, the more dissimilar the non-UIP disease subtype may be from UIP. A comparison of the list of differential genes in each non-UIP subtype with that from all non-UIP samples may show that the number of overlapping genes may be highly dependent on the number of differential genes identified in the individual non-UIP subtype, indicating that some non-UIP diseases may have more dominant effects on the overall differential genes found between all non-UIP and UIP samples (Table 15). Moreover, there may be few overlapping differential genes among those identified in individual non-UIP diseases. For example, 172 genes may be common between 1174 differential genes in Sarcoidosis and 701 in RB, and 6 common genes may be found among differential genes from sarcoidosis, RB and NSIP. There may be no common genes among differential genes from bronchiolitis, NSIP and HP. This may suggest distinct molecular expression patterns within diseases in non-UIP samples.

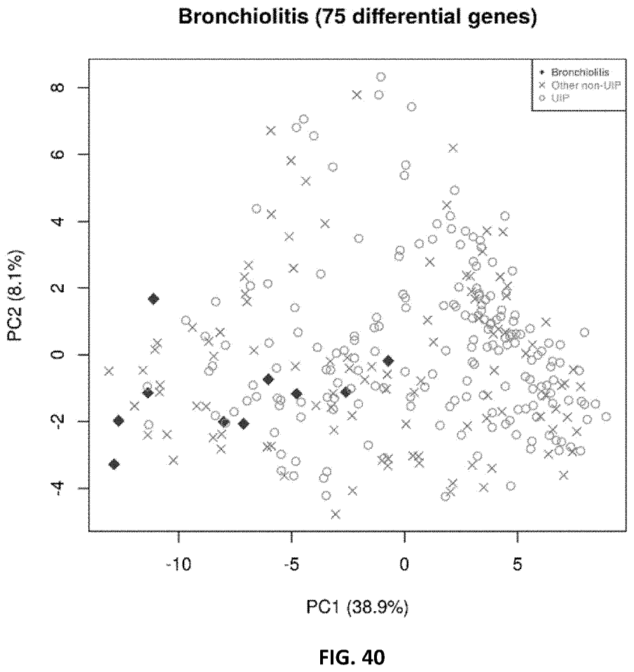

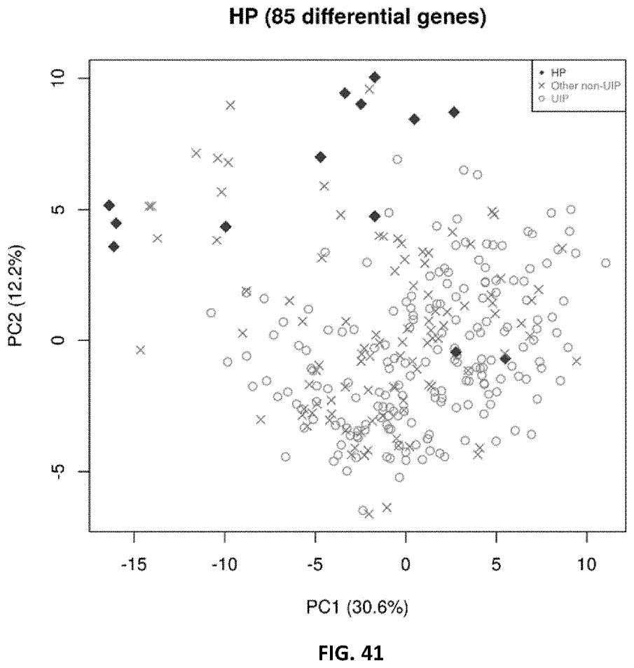

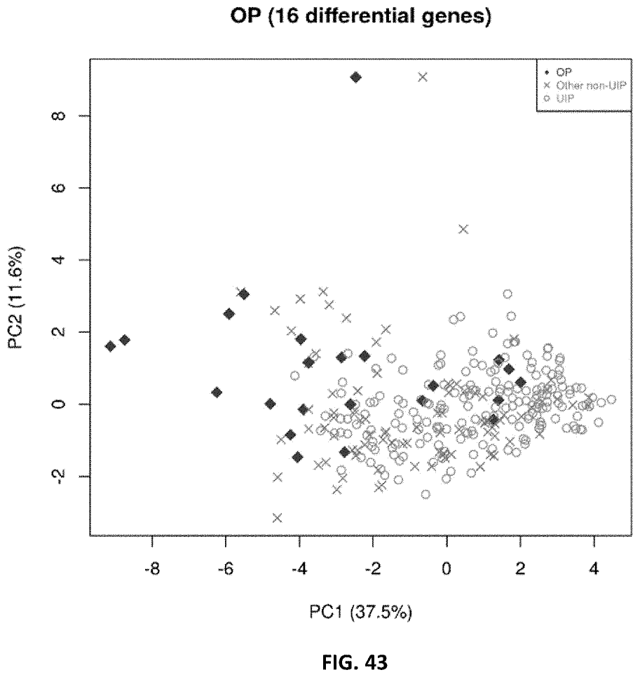

The PCA plot using the differentially expressed genes between a non-UIP subtype and UIP samples may show that the specific non-UIP disease subtype may tend to be well-separated from UIP samples for diseases such as RB and HP (FIG. 39 and FIG. 41), but other non-UIP samples may be interspersed with UIP samples (FIG. 40 and FIG. 43). This may demonstrate that differential genes derived from one non-UIP subtype may not be generalizable to other non-UIP diseases.

Comparison Between in Silico Mixing and In Vitro Pooling within Patient

In silico mixed samples within each patient may be used to model in vitro pooled samples for evaluation within the training set. To ensure in silico mixed and in vitro pooled samples may be reasonably matched, the pooled samples of 11 patients may be sequenced and compared with in silico mixed samples. The average r-squared based on expression level of 26,268 genes for the pairs of in silico mixed and in vitro pooled samples may be 0.99 (SD=0.003), which may indicate that the simulated expression level of in silico mixed samples may be well-matched with that of in vitro pooled samples, considering the average r-squared values may be 0.98 (SD=0.008) for technical replicates and 0.94 (0.04) for biological replicates.

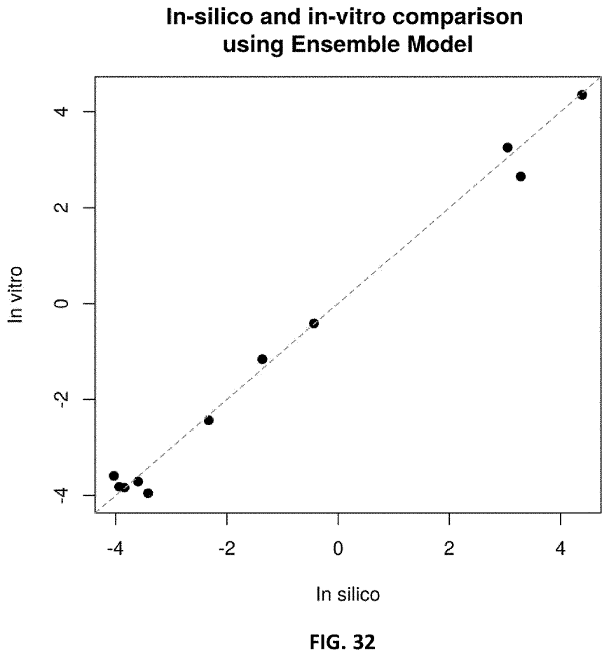

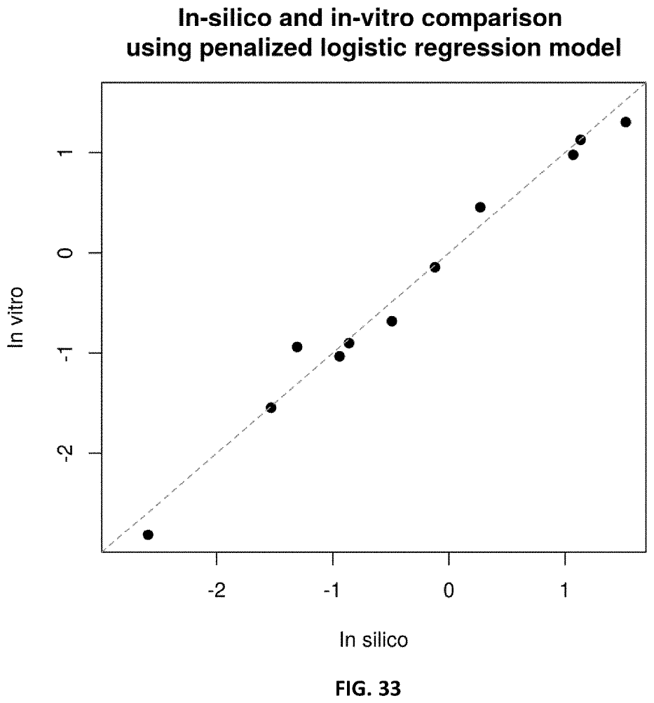

The classification scores of in silico and in vitro mixed samples by two candidate classifiers, the ensemble and penalized logistic regression models (described below) may also be compared in a scatterplot (FIG. 32 and FIG. 33). The number of replicates for each in vitro pooled sample may range from 3 to 5, so the mean score of the multiple replicates may be used. The classification scores of in silico mixed samples may be highly correlated with those of in vitro pooled samples with Pearson's correlation of 0.99 for both classifiers (FIG. 32 and FIG. 33). The points may fall right around the line of X=Y with no obvious shift or rotation.

Cross-Validation Performance on the Training Set

Multiple methods of feature selection and machine learning algorithms on training set of 354 TBB samples from 90 patients may be evaluated. As an initial attempt, individual methods and ensemble models may be evaluated separately based on 5-fold CV and cross-validated AUC (cvAUC) as estimated using the mean of the empirical AUC of each fold. Overall, the linear models such as the penalized regression model (cvAUC=0.89) may outperform non-linear tree-based models, such as random forest (cvAUC=0.83) and gradient boosting (cvAUC=0.84). The cvAUC of a neural network classifier may be under 0.8. The best performance may be achieved by (1) the ensemble model of SVMs with linear and radial kernels, and (2) penalized logistic regression; both of which have cvAUC=0.89. However, with the heterogeneity among diseases and the small samples size, CV performance on all models may be found to vary significantly depending on the split.

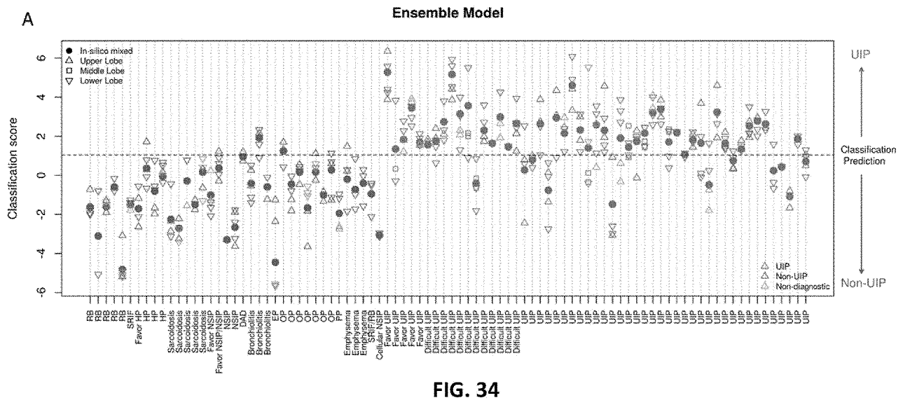

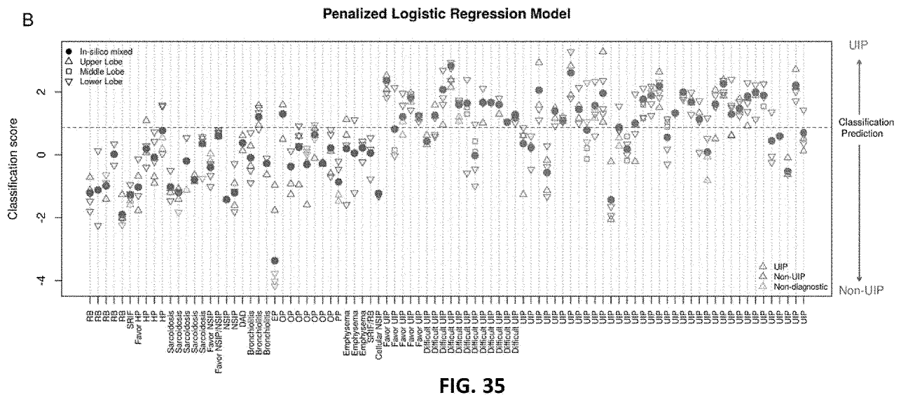

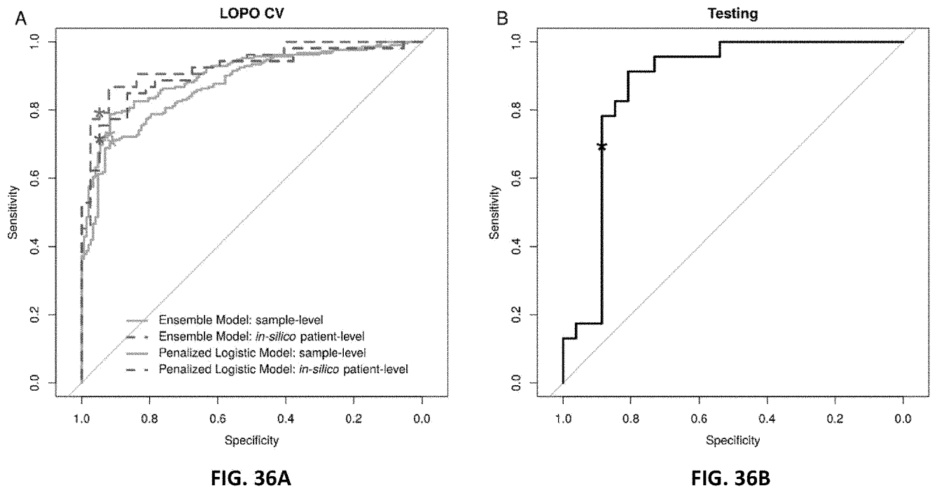

In LOPO CV, the patient-level performance may be evaluated by using 100 replicates of in silico mixed samples for each patient within LOPO CV folds. The computed classification scores of individual samples and averaged scores of in silico mixed samples may be shown in FIG. 34 and FIG. 35. Overall, the patient-level performance may be slightly higher compared to the sample-level performance. Based on combined scores across LOPO CV folds, the ensemble model and the penalized logistic regression model may achieve the best performance with an AUC of 0.9 [0.87-0.93] and 0.87 [0.83-0.91] at sample-level and 0.93 [0.88-0.98] and 0.91 [0.85-0.97] at in silico mixing patient-level, respectively (FIG. 36A).

Robustness of Classifiers

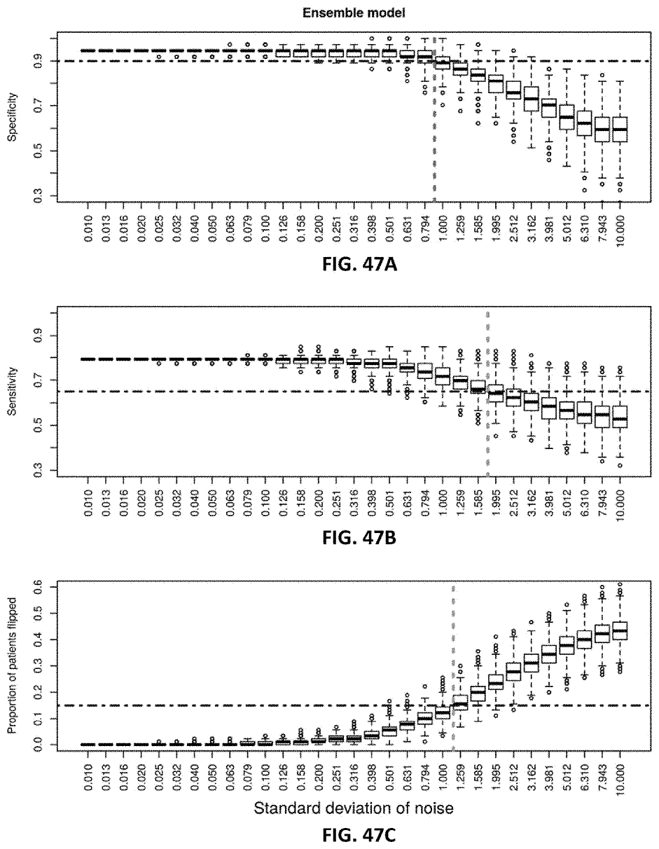

The estimated score variability may be 0.46 and 0.22 for the ensemble model and the penalized logistic regression model, respectively (Table 16). Both may be less than 0.9 and 0.48, the pre-specified thresholds of acceptable score variability (FIG. 47A-C and FIG. 48A-C). Considering the score range of the ensemble classifier may be wider than the penalized logistic regression classifier, the proportion of the variability to the range of 5% and 95% quantiles of scores may be compared. Overall, the penalized logistic regression classifier may have less variability in scores than the ensemble model. This may imply that the penalized logistic regression may be more robust to the technical (reagent/laboratory) batch effects and may offer more consistent scores for technical replicates. (Table 16). With high cross-validation performance and robustness, the penalized logistic regression model may be chosen as our final candidate model for the independent validation.

Independent Validation Performance

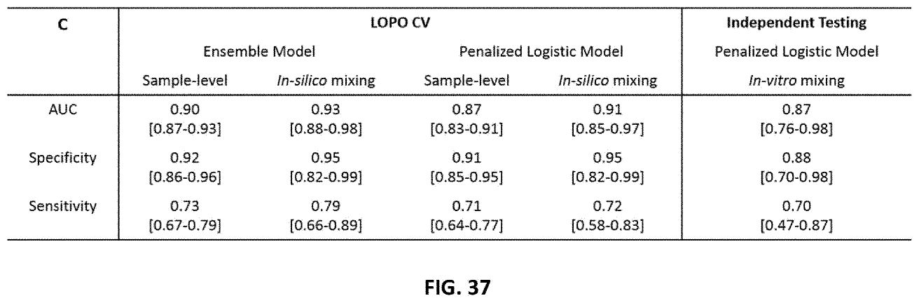

Using the locked penalized logistic classifier with a pre-specified decision boundary, 0.87, the validation performance may be evaluated based on the independent test set of in vitro mixed samples. The final classifier may achieve specificity 0.88 [0.70-0.98] and sensitivity 0.70 [0.47-0.87] with AUC 0.87 [0.76-0.98] (FIG. 36B and FIG. 37). The point estimate of the validation performance may be lower than in silico patient-level training CV performance, but with p-values, 0.6, 0.7, and 1 for AUC, sensitivity and specificity, respectively, indicating negligible difference.

Discussion

In this study, accurate and robust classification may be achievable even when critical challenges exist. By leveraging appropriate statistical methodologies, machine learning approaches, and RNA sequencing technology, a meaningful diagnostic test may be provided to improve the care of patients with interstitial lung diseases.

Machine learning, particularly deep learning, may have experienced revolutionary progress in the last few years. Empowered with these recently developed and highly sophisticated tools, classification performance may be dramatically improved in many applications [Lecun et al, which is entirely incorporated herein by reference]. However, most of these tools may require readily available and high-confidence labels as well as large sample size: the magnitude of the performance improvement may be directly and positively related with the number of samples with high-quality labels [Gu et al and Sun et al, which are entirely incorporated herein by reference]. In this project, like many other clinical studies based on patient samples, the sample size may be limited: for example, 90 patients in the training set (Table 14). On top of that, the non-UIP group may not be one physiologically homogenous disease, but rather a collection of many types of diseases, each with its own distinct biology, several of which may have only one or two patients in the training set [Libbrecht et al, which is entirely incorporated herein by reference] (Table 14). Not surprisingly, these various types of non-UIP diseases may be not only physiologically distinct, but may be also different at the molecular and genomic level. The training samples may be utilized to identify common features across non-UIP diseases in respect to differentiating from the UIP group may be tried but none emerged (Table 15, FIG. 38). Furthermore, three or more disease types (Amyloid or light chain deposition, Exogenous lipid pneumonia, and Organizing alveolar hemorrhage) may present in the test set and may not be encountered in the training set (Table 14). A change in UIP proportions may also be observed between training (59%) and testing (47%). The last two factors may help explain the slightly lower performance in the test set as compared to the cross-validation performance of the training set. Recent advances in machine learning that leverage large sample size may not be applicable in this situation. In some case, a focus may be on more traditional linear models or tree-based models. It may also explain among candidates, why linear models may outperform non-linear tree-based models because a sample size in individual non-UIP disease groups may be too small to power any interaction the tree-model may be trying to capture.

To directly address the small training size, up to 5 distinct TBB samples within the same patient may be run from RNA extraction through sequencing to successfully expand the 90 patient set to encompass 354 samples (Table 14). This, in concept, may be similar to the data augmentation idea, but instead of simulating or extrapolating the augmented data, sequencing data may be generated from real experiments on multiple TBB samples from the same patient. The goal may be to provide additional information to enhance classification performance. Special caution may be taken to use patient as the smallest unit when defining the cross-validation fold and evaluating performance. This may prevent patients with more samples from having higher weight, or samples from the same patient straddling on both side of model building and model evaluation, causing over-fitting. A nested cross-validation may also be applied as well as the one SD (standard deviation) rule for model selection and parameter optimization to correctly factor-in the high variability on performance due to small sample size and to aggressively trim down the model complexity to guard against overfitting.

While running multiple TBB samples per patient in the training set may help with the sample size limitation, it may create a new problem. In the commercial setting, it may be economically viable only if it may be limited to test one sequencing run per patient. To achieve that, RNA material from multiple TBB samples within one patient may need to be pooled together before sequencing. However, whether a classifier trained on individual TBB samples may be applicable to pooled TBB samples may become a critical question that may need to be addressed before setting off the validation experiment. To answer this question, a series of in-silico mixing simulations may be performed to mimic patient-level in-vitro pools of the test set. This approach may also be the fundamental building block for defining the prospective decision boundary of the classifier as well as the optimal number of TBBs required to achieve the best classification performance [Pankratz et al]. The simulated in-silico data may agree well with the experimental in-vitro data (FIGS. 32 and 33) giving confidence in using this approach to extrapolate expected performance to pooled samples and proceed with the validation experiments with the pooled setting. This in silico approach may work well in this example since samples pooled together may be of the same type (TBB) and from the same patient, thus have similar characteristics such as the rate of duplicated reads or the total number of reads. However, it may be tricky to extend the proposed in-silico mixing model to mix samples of different characteristics or qualities, for example UIP vs non-UIP samples or TBB mixed with different type of samples such as blood. In those cases, samples with substantially higher total number of reads may tend to dominate the expressions of combined samples violating the basic assumptions of the mixed model proposed here. More sophisticated methodology may be required to accurately model such complex procedures and biological interaction.

A successful validation that may meet the required clinical performance (FIG. 36A-B and FIG. 37) may be the first step towards a useful commercial product aiming to improve patient care. Equally important, but often overlooked, may be the importance of providing consistent and reliable performance for the future patient stream. This may require proactive anticipation to address any potential batch effects of sequencing data from incoming patients that may cause systematic changes in classification scores and result in false clinical predictions. This important issue may be tackled starting from the upstream feature selection (FIG. 39-44) where genes that may be highly sensitive to batch effects may be removed from any downstream analysis. Furthermore, additional experimental data may be generated for 10 distinct TBB samples in three different batches; none of the batches may be used in generating training samples. This experiment may be leveraged to directly evaluate each candidate model's robustness against unseen batches and may help select the final model. However, experimental data may evaluate a finite number of batches. Thus, to anticipate unforeseen changes, a monitoring scheme may be developed based on control samples run in each of the commercial plate/batch to detect any unexpected potential changes. If such unexpected changes may occur, a normalization method that may directly addresses batch correction may be necessary to map new scores to the space of validation classification scores.

Conclusions

Limited sample size and high heterogeneity within the non-UIP class may be two major classification challenges faced in this example and which may commonly exist in clinical studies. In addition, a successful commercial product may need to perform economically and consistently for all future incoming samples, which may require the underlying classification model to be applicable to pooled samples and highly robust against assay variability. It may be feasible to achieve highly accurate and robust classification despite these difficulties. The methodologies may have proven to be successful in this example and may be applicable to other clinical scenarios facing similar difficulties.

### Example 2—Molecular Profiling and Cytological Examination

An individual is symptomatic for lung cancer. The individual consults her primary care physician who examines the individual and refers her to an endocrinologist. The endocrinologist obtains a sample via bronchoscopy, and sends the sample to a cytological testing laboratory. The cytological testing laboratory performs routine cytological testing on a portion of the bronchoscopy, the results of which are suspicious or ambiguous (i.e., indeterminate). The cytological testing laboratory suggests to the endocrinologist that the remaining sample may be suitable for molecular profiling, and the endocrinologist agrees.

The remaining sample is analyzed using the methods and compositions herein. The results of the molecular profiling analysis suggest a high probability of early stage lung cancer. The results further suggest that molecular profiling analysis combined with patient data. The endocrinologist reviews the results and prescribes the recommended therapy.

The cytological testing laboratory bills the endocrinologist for routine cytological tests and for the molecular profiling. The endocrinologist remits payment to the cytological testing laboratory and bills the individual's insurance provider for all products and services rendered. The cytological testing laboratory passes on payment for molecular profiling to the molecular profiling business and withholds a small differential.

### Example 3

A subject is at-risk for lung cancer due to exposure to second-hand smoke. The subject is asymptomatic for lung cancer. A medical professional obtains a nasal tissue sample from the subject. A molecular classifier as described herein analyzes the nasal tissue sample. Based on a presence or absence of a plurality of biomarkers, a medical professional recommends the subject to receive a low-dose CT scan or recommends analyzing another nasal tissue sample 1 year later using the molecule classifier.

### Example 4

A subject has previously received confirmation of a presence of a lung nodule. A medical professional obtains a nasal tissue sample from the subject. A molecular classifier as described herein analyzes the nasal tissue sample. Based on a presence or absence of a plurality of biomarkers, a medical professional recommends the subject to receive a bronchoscopy or recommends analyzing another nasal tissue sample 1 year later using the molecular classifier.

### Example 5

A subject is currently receiving an interventive therapy. A medical professional obtains a nasal tissue sample from the subject. A molecular classifier as described herein analyzes the nasal tissue sample. Based on a presence or absence of a plurality of biomarkers, a medical professional recommends the subject continue the interventive therapy or stop the interventive therapy and begin a different interventive therapy.

### Example 6

A subject has previously received a surgical resection of a malignant tumor. A medical professional obtains a nasal tissue sample from the subject. A molecular classifier as described herein analyzes the nasal tissue sample. Based on a presence or absence of a plurality of biomarkers, a medical professional recommends a treatment regime for the subject or recommends analyzing another nasal tissue sample 1 year later using the molecular classifier.

### Computer Control Systems

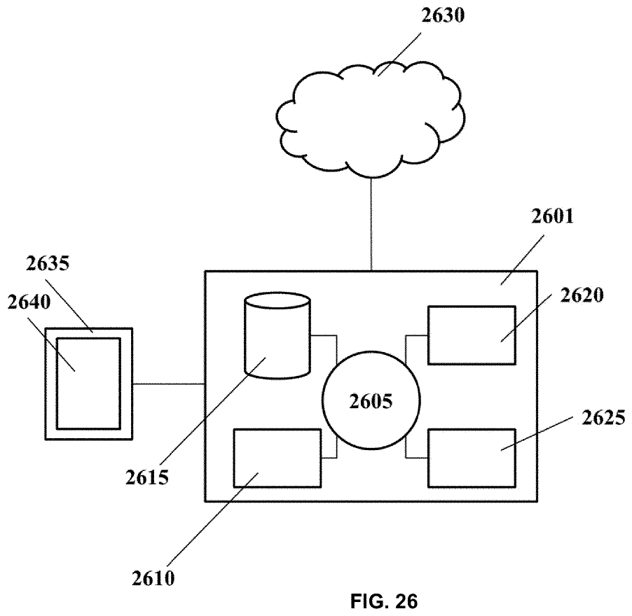

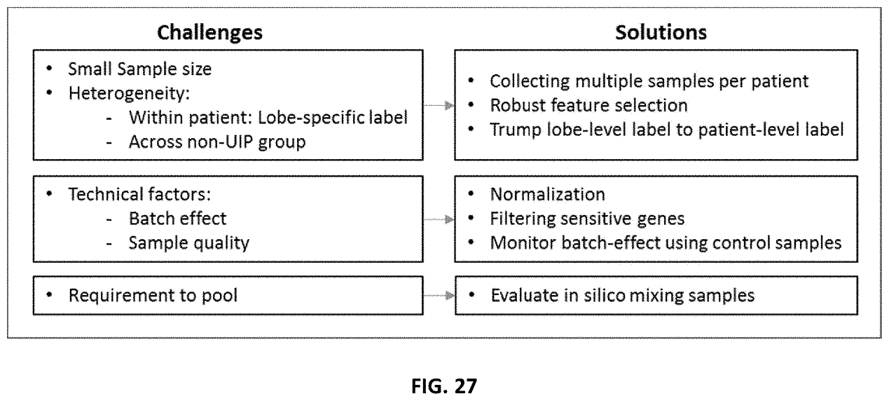

The present disclosure provides computer control systems that are programmed to implement methods of the disclosure. FIG. 26 shows a computer system 2601 that is programmed or otherwise configured to implement the methods provided herein. The computer system 2601 can regulate various aspects of diagnosing a lung condition in a subject, predicting a risk of developing a lung condition in a subject, predicting an efficacy of treatment in a subject having a lung condition, or combinations thereof of the present disclosure, such as, for example, (i) comparing one or more biomarkers of a sample to a reference set of biomarkers, (ii) training an algorithm to develop a classifier, (iii) applying a classifier to make a diagnosis, a prediction, or a recommendation based on a sample input, or (iv) any combination thereof. The computer system 2601 can be an electronic device of a user or a computer system that is remotely located with respect to the electronic device. The electronic device can be a mobile electronic device.

The computer system 2601 includes a central processing unit (CPU, also “processor” and “computer processor” herein) 2605, which can be a single core or multi core processor, or a plurality of processors for parallel processing. The computer system 2601 also includes memory or memory location 2610 (e.g., random-access memory, read-only memory, flash memory), electronic storage unit 2615 (e.g., hard disk), communication interface 2620 (e.g., network adapter) for communicating with one or more other systems, and peripheral devices 2625, such as cache, other memory, data storage and/or electronic display adapters. The memory 2610, storage unit 2615, interface 2620 and peripheral devices 2625 are in communication with the CPU 2605 through a communication bus (solid lines), such as a motherboard. The storage unit 2615 can be a data storage unit (or data repository) for storing data. The computer system 2601 can be operatively coupled to a computer network (“network”) 2630 with the aid of the communication interface 2620. The network 2630 can be the Internet, an internet and/or extranet, or an intranet and/or extranet that is in communication with the Internet. The network 2630 in some cases is a telecommunication and/or data network. The network 2630 can include one or more computer servers, which can enable distributed computing, such as cloud computing. The network 2630, in some cases with the aid of the computer system 2601, can implement a peer-to-peer network, which may enable devices coupled to the computer system 2601 to behave as a client or a server.

The CPU 2605 can execute a sequence of machine-readable instructions, which can be embodied in a program or software. The instructions may be stored in a memory location, such as the memory 2610. The instructions can be directed to the CPU 2605, which can subsequently program or otherwise configure the CPU 2605 to implement methods of the present disclosure. Examples of operations performed by the CPU 2605 can include fetch, decode, execute, and writeback.

The CPU 2605 can be part of a circuit, such as an integrated circuit. One or more other components of the system 2601 can be included in the circuit. In some cases, the circuit is an application specific integrated circuit (ASIC).

The storage unit 2615 can store files, such as drivers, libraries and saved programs. The storage unit 2615 can store user data, e.g., user preferences and user programs. The computer system 2601 in some cases can include one or more additional data storage units that are external to the computer system 2601, such as located on a remote server that is in communication with the computer system 2601 through an intranet or the Internet.

The computer system 2601 can communicate with one or more remote computer systems through the network 2630. For instance, the computer system 2601 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2601 via the network 2630.

Methods as described herein can be implemented by way of machine (e.g., computer processor) executable code stored on an electronic storage location of the computer system 2601, such as, for example, on the memory 2610 or electronic storage unit 2615. The machine executable or machine readable code can be provided in the form of software. During use, the code can be executed by the processor 2605. In some cases, the code can be retrieved from the storage unit 2615 and stored on the memory 2610 for ready access by the processor 2605. In some situations, the electronic storage unit 2615 can be precluded, and machine-executable instructions are stored on memory 2610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2601 can include or be in communication with an electronic display 2635 that comprises a user interface (UI) 2640 for providing, for example, an output or readout of the classifier or trained algorithm. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2605. The algorithm can, for example, (i) determine a presence or one or more biomarkers in a sample compared to a reference set of biomarkers.

### REFERENCES

Flaherty K R, King T E, Jr., Raghu G, Lynch J P, 3rd, Colby T V, Travis W D, Gross B H, Kazerooni E A, Toews G B, Long Q, et al: Idiopathic interstitial pneumonia: what is the effect of a multidisciplinary approach to diagnosis? Am J Respir Crit Care Med 2004, 170:904-910.
Travis W D, Costabel U, Hansell D M, King T E, Jr., Lynch D A, Nicholson A G, Ryerson C J, Ryu J H, Selman M, Wells A U, et al: An official American Thoracic Society/European Respiratory Society statement: Update of the international multidisciplinary classification of the idiopathic interstitial pneumonias. Am J Respir Crit Care Med 2013, 188:733-748.
Flaherty K R, Andrei A C, King T E, Jr., Raghu G, Colby T V, Wells A, Bassily N, Brown K, du Bois R, Flint A, et al: Idiopathic interstitial pneumonia: do community and academic physicians agree on diagnosis? Am J Respir Crit Care Med 2007, 175:1054-1060.
Tuch B B, Laborde R R, Xu X, Gu J, Chung C B, Monighetti C K, Stanley S J, Olsen K D, Kasperbauer J L, Moore E J, et al: Tumor transcriptome sequencing reveals allelic expression imbalances associated with copy number alterations. PLoS One 2010, 5:e9317.
Twine N A, Janitz K, Wilkins M R, Janitz M: Whole transcriptome sequencing reveals gene expression and splicing differences in brain regions affected by Alzheimer's disease. PLoS One 2011, 6:e16266.
Boyle E A, Li Y I, Pritchard J K: An Expanded View of Complex Traits: From Polygenic to Omnigenic. Cell 2017, 169:1177-1186.
Pankratz D G, Choi Y, Imtiaz U, Fedorowicz G M, Anderson J D, Colby T V, Myers J L, Lynch D A, Brown K K, Flaherty K R, et al: Usual Interstitial Pneumonia Can Be Detected in Transbronchial Biopsies Using Machine Learning. Ann Am Thorac Soc 2017.
Sorlie T, Tibshirani R, Parker J, Hastie T, Marron J S, Nobel A, Deng S, Johnsen H, Pesich R, Geisler S, et al: Repeated observation of breast tumor subtypes in independent gene expression data sets. Proc Natl Acad Sci USA 2003, 100:8418-8423.
Brennan C W, Verhaak R G, McKenna A, Campos B, Noushmehr H, Salama S R, Zheng S, Chakravarty D, Sanborn J Z, Berman S H, et al: The somatic genomic landscape of glioblastoma. Cell 2013, 155:462-477.
Kim S Y, Diggans J, Pankratz D, Huang J, Pagan M, Sindy N, Tom E, Anderson J, Choi Y, Lynch D A, et al: Classification of usual interstitial pneumonia in patients with interstitial lung disease: assessment of a machine learning approach using high-dimensional transcriptional data. Lancet Respir Med 2015, 3:473-482.
Dobin A, Davis C A, Schlesinger F, Drenkow J, Zaleski C, Jha S, Batut P, Chaisson M, Gingeras T R: STAR: ultrafast universal RNA-seq aligner. Bioinformatics 2013, 29:15-21.
Anders S, Pyl P T, Huber W: HTSeq—a Python framework to work with high-throughput sequencing data. Bioinformatics 2015, 31:166-169.
DeLuca D S, Levin J Z, Sivachenko A, Fennell T, Nazaire M D, Williams C, Reich M, Winckler W, Getz G: RNA-SeQC: RNA-seq metrics for quality control and process optimization. Bioinformatics 2012, 28:1530-1532.
Love M I, Huber W, Anders S: Moderated estimation of fold change and dispersion for RNA-seq data with DESeq2. Genome Biol 2014, 15:550.
Anders S, McCarthy D J, Chen Y, Okoniewski M, Smyth G K, Huber W, Robinson M D: Count-based differential expression analysis of RNA sequencing data using R and Bioconductor. Nat Protoc 2013, 8:1765-1786.
Dobson A J, Barnett A: An introduction to generalized linear models. CRC press; 2008.
Krstajic D, Buturovic L J, Leahy D E, Thomas S: Cross-validation pitfalls when selecting and assessing regression and classification models. J Cheminform 2014, 6:10.
Friedman J, Hastie T, Tibshirani R: The elements of statistical learning. Springer series in statistics New York; 2001.
LeCun Y, Bengio Y, Hinton G: Deep learning. Nature 2015, 521:436-444.
Gu B, Hu F, Liu H: Modelling classification performance for large data sets. Advances in Web-Age Information Management 2001:317-328.
Sun C, Shrivastava A, Singh S, Gupta A: Revisiting Unreasonable Effectiveness of Data in Deep Learning Era. arXiv preprint arXiv:170702968 2017.
Libbrecht M W, Noble W S: Machine learning applications in genetics and genomics. Nat Rev Genet 2015, 16:321-332.
Wong S C, Gatt A, Stamatescu V, McDonnell M D: Understanding data augmentation for classification: when to warp? In. IEEE; 2016: 1-6; arXiv:1609.08764.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for screening a subject for a lung condition, comprising:
(a) assaying epithelial tissue from a first sample obtained from a subject that has been (1) computer analyzed for a presence of one or more risk factors for developing said lung condition and (2) identified with said presence of said one or more risk factors, to identify a presence or absence of one or more biomarkers associated with a risk of developing said lung condition in said first sample; and
(b) upon identifying said presence or absence of said one or more biomarkers, (i) directing an electronic imaging scan of a lung region of said subject to be obtained, which lung region is suspected of exhibiting said lung condition, or (ii) assaying other epithelial tissue from a second sample of said subject.

2. (canceled)

3. The method of claim 1, wherein said electronic imaging scan is a low-dose computerized tomography (LDCT) scan or magnetic resonance imaging (MM).

4. (canceled)

5. The method of claim 1, wherein said lung condition is lung cancer, chronic obstructive pulmonary disease (COPD), interstitial lung disease (ILD), or any combination thereof.

6. The method of claim 1, wherein said lung condition is a lung cancer, and wherein said lung cancer comprises: a non-small cell lung cancer; an adenocarcinoma; a squamous cell carcinoma; a large cell carcinoma; a small cell lung cancer; or any combination thereof.

7. The method of claim 1, wherein said first sample or said second sample is obtained by a bronchoscopy, bronchial brushing, or nasal brushing.

8. (canceled)

9. The method of claim 1, wherein said first sample or said second sample comprises a mucous epithelial tissue, a nasal epithelial tissue, a lung epithelial tissue, or any combination thereof.

10. The method of claim 1, wherein said first sample or said second sample comprises epithelial tissue obtained along an airway of said subject.

11. The method of claim 1, wherein a portion of said first sample or said second sample is subjected to cytological testing that identifies said first sample or said second sample as ambiguous or suspicious.

12. The method of claim 11, wherein upon identifying said first sample or said second sample as ambiguous or suspicious, performing (b) on a second portion of said sample, which second portion comprises said epithelial tissue.

13. (canceled)

14. The method of claim 1, wherein said second sample is a different sample type from said first sample.

15. The method of claim 1, wherein said first sample is obtained from said subject at a first time point and said second sample is obtained from said subject at a second time point, wherein said second time point is after said first time point.

16. (canceled)

17. The method of claim 1, wherein (a) comprises comparing said presence or absence of said one or more biomarkers to a reference set of one or more biomarkers.

18. (canceled)

19. (canceled)

20. (canceled)

21. (canceled)

22. (canceled)

23. The method of claim 1, wherein said one or more risk factors comprise: smoking; exposure to environmental smoke; exposure to radon; exposure to air pollution; exposure to radiation; exposure to an industrial substance; inherited or environmentally-acquired gene mutations; a subject's age; a subject having a secondary health condition; or any combination thereof.

24. (canceled)

25. (canceled)

26. The method of claim 1, wherein said one or more biomarkers comprise one or more of: a gene or fragment thereof; a sequence variant; a fusion; a mitochondrial transcript; an epigenetic modification; a copy number variation; a loss of heterozygosity (LOH); or any combination thereof.

27. The method of claim 1, wherein said presence or absence of said one or more biomarkers comprises a level of expression.

28. The method of claim 1, wherein said method identifies whether said subject is at an increased risk for developing said lung condition.

29. The method of claim 1, wherein said identifying of (b) comprises employing a trained algorithm.

30. The method of claim 29, wherein said trained algorithm is trained by a training set comprising epithelial cells obtained from an airway of an individual.

31. The method of claim 29, wherein said trained algorithm is trained by a training set comprising samples benign for said lung condition and samples malignant for said lung condition.

32. The method of claim 29, wherein said trained algorithm is trained by a training set comprising samples obtained from subjects having one or more risk factors.

33.-60. (canceled)
